(19) United States
(12) Patent Application Publication
(10) Pub. No.: US 2019/0041401 A1
(43) Pub. Date: Feb. 7, 2019

(54) Methods of Treating Subjects having Severe and/or Persistent Asthma

(71) Applicant: National Jewish Health, Denver, CO (US)

(72) Inventor: Rafeul Alam, Aurora, CO (US)

(21) Appl. No.: 16/165,402

(22) Filed: Oct. 19, 2018

Related U.S. Application Data
(62) Division of application No. 15/080,972, filed on Mar. 25, 2016, now abandoned.
(60) Provisional application No. 62/138,262, filed on Mar. 25, 2015.

(51) Int. Cl. G01N 33/68; C12Q 1/6883

(52) U.S. Cl. CPC G01N 33/6884; G01N 2333/54; G01N 2800/122; G01N 2800/52; C12Q 2600/158; C12Q 1/6883; C12Q 2600/106

(57) ABSTRACT

The present invention is directed toward novel methods to treat subjects having severe and/or persistent asthma.

### CROSS-REFERENCE TO RELATED APPLICATION

This application is a divisional application of U.S. application Ser. No. 15/080,972, filed Mar. 25, 2016 which claims the benefit of priority under 35 U.S.C. § 119(e) to U.S. Provisional Patent Application No. 62/138,262, filed Mar. 25, 2015. The entire disclosure of U.S. application Ser. No. 15/080,972 and U.S. Provisional Patent Application No. 62/138,262 are incorporated herein by reference.

### GOVERNMENT SUPPORT

This invention was made with government support under grant number R01 AI091614A received from the National Institute of Allergic and Infectious Diseases (NIAID). The government has certain rights in the invention.

### REFERENCE TO A SEQUENCE LISTING

This application contains a Sequence Listing submitted electronically as a text file by EFS-Web. The text file, named “2879-186_SequenceListing_ST25”, has a size in bytes of 5 KB, and was recorded on 25 Mar. 2016. The information contained in the text file is incorporated herein by reference in its entirety pursuant to 37 CFR § 1.52(e)(5).

### FIELD OF THE INVENTION

The present invention is directed toward novel methods to identify, predict and/or treat subjects having severe and/or persistent asthma.

### BACKGROUND OF THE INVENTION

Allergic asthma is characterized by a T helper 2 (Th2) immune response and the presence of allergen-specific Immunoglobulin E (IgE) antibodies (Robinson D S, et al. Predominant TH2-like bronchoalveolar T-lymphocyte population in atopic asthma. N Engl J Med 1992; 326:298-304; Burrows B, et al. Association of asthma with serum IgE levels and skin-test reactivity to allergens. N Engl J Med 1989; 320:271-7). IgE mediated mast cell activation plays an important role in asthmatic patients. This conclusion is supported by the efficacy of omalizumab in many patients with refractory asthma (Strunk R C, and Bloomberg G R. Omalizumab for asthma. N Engl J Med 2006; 354: 2689-95). The differentiation of T cells into various T helper cell populations is a functional but not terminal differentiation state. This was clearly demonstrated in early studies by Coffman and colleagues, who showed that fully differentiated Th2 cells could produce Th1 cytokines in the presence of appropriate stimulants (Coffman R L, et al. Reversal of polarized T helper 1 and T helper 2 cell populations in murine leishmaniasis. Ciba Found Symp 1995; 195:20-3; Mocci S, and Coffman R L. Induction of a Th2 population from a polarized Leishmania-specific Th1 population by in vitro culture with IL-4. J Immunol 1995; 154:3779-87.3). In recent years, this plasticity of T helper cell function has drawn attention (Yang X O, et al. Molecular antagonism and plasticity of regulatory and inflammatory T cell programs. Immunity 2008; 29:44-56; Koenen H J, et al. Human CD25highFoxp3pos regulatory T cells differentiate into IL-17-producing cells. Blood 2008; 112:2340-52; Lee Y K, et al. Late developmental plasticity in the T helper 17 lineage. Immunity 2009; 30:92-107; Wei G, et al. Global mapping of H3K4me3 and H3K27me3 reveals specificity and plasticity in lineage fate determination of differentiating CD41 T cells. Immunity 2009; 30:155-67; Zhou L, et al. Plasticity of CD41 T cell lineage differentiation. Immunity 2009; 30:646-55). Studies have shown interconversion of T helper cells, especially interconversion of regulatory T and Th17 cells. Furthermore, studies have demonstrated the presence of dual-positive Th2/Th17 cells in the blood and tissue of asthmatic patients (Cosmi L, et al. Identification of a novel subset of human circulating memory CD4(1) T cells that produce both IL-17A and IL-4. J Allergy Clin Immunol 2010; 125:222-30, e1-4; Wang Y H, et al. A novel subset of CD4(1) T(H)2 memory/effector cells that produce inflammatory IL-17 cytokine and promote the exacerbation of chronic allergic asthma. J Exp Med 2010; 207:2479-91) and healthy subjects (Malmhall C, et al. Immunophenotyping of circulating T helper cells argues for multiple functions and plasticity of T cells in vivo in humans-possible role in asthma. PLoS One 2012; 7:e40012).

Although allergens play an important role, there are other environmental factors, such as infection, and a broad range of chemical and physical factors that contribute to exacerbation of asthma. Many of the latter factors are likely to elicit a Th17-type immune response. A particular matter of interest is the qualitative difference between Th2 and Th17 cells in their response to glucocorticoids. IL-17 production by Th17 cells has been shown to be less susceptible to inhibition by glucocorticoids compared with IL-4 and IL-5 production by Th2 cells (McKinley L, et al. TH17 cells mediate steroid-resistant airway inflammation and airway hyperresponsiveness in mice. J Immunol 2008; 181:4089-97). Thus the emergence of Th2/Th17 cells in the airways could make asthma less responsive to glucocorticoid treatment. A number of articles have reported increased presence of IL-17 in lung biopsy specimens and sputum from asthmatic patients (Pene J, et al. Chronically inflamed human tissues are infiltrated by highly differentiated Th17 lymphocytes. J Immunol 2008; 180:7423-30; Barczyk A, et al. Interleukin-17 in sputum correlates with airway hyperresponsiveness to methacholine. Respir Med 2003; 97:726-33; Bullens D M, et al. IL-17 mRNA in sputum of asthmatic patients: linking T cell driven inflammation and granulocytic influx? Respir Res 2006; 7:135; Molet S, et al. IL-17 is increased in asthmatic airways and induces human bronchial fibroblasts to produce cytokines. J Allergy Clin Immunol 2001; 108:430-8; Al-Ramli W, et al. T(H)17-associated cytokines (IL-17A and IL-17F) in severe asthma. J Allergy Clin Immunol 2009; 123:1185-7; Agache I, et al. Increased serum IL-17 is an independent risk factor for severe asthma. Respir Med 2010; 104:1131-7; Wong C K, et al. Proinflammatory cytokines (IL-17, IL-6, IL-18 and IL-12) and Th cytokines (IFN-gamma, IL-4, IL-10 and IL-13) in patients with allergic asthma. Clin Exp Immunol 2001; 125:177-83; Vazquez-Tello A, et al. Induction of glucocorticoid receptor-beta expression in epithelial cells of asthmatic airways by T-helper type 17 cytokines. Clin Exp Allergy 2010; 40:1312-22). Increased expression of IL-17 was associated with severe asthma. However, there are no reports on the presence of dual-positive Th2/Th17 cells in bronchoalveolar lavage (BAL) fluid from asthmatic patients. Thus there are a number of reasons to study and determine the significance of Th2/Th17 cells in asthmatic patients.

In addition, asthma is a chronic illness (Jackson D J, et al. Asthma exacerbations: origin, effect, and prevention. J Allergy Clin Immunol 2011; 128:1165-74; Lemanske R F, and Busse W W. Asthma: clinical expression and molecular mechanisms. J Allergy Clin Immunol 2010; 125(Suppl):S95-102) with airway hyperreactivity and remodeling persisting in asymptomatic patients with normal pulmonary function (Townley R G, et al. Bronchial sensitivity to methacholine in current and former asthmatic and allergic rhinitis patients and control subjects. J Allergy Clin Immunol 1975; 56:429-42; Shapiro G G, et al. Methacholine bronchial challenge in children. J Allergy Clin Immunol 1982; 69:365-9). Because of the perennial nature of some allergens, it has been difficult to ascertain whether continuous allergen exposure is necessary for the persistence of asthma. Longitudinal observations in patients with occupational asthma indicate that asthma persists in most patients years after removal from occupational exposure (Malo J L, et al. Natural history of occupational asthma: relevance of type of agent and other factors in the rate of development of symptoms in affected subjects. J Allergy Clin Immunol 1992; 90:937-44; Moller D R, et al. Persistent airways disease caused by toluene diisocyanate. Am Rev Respir Dis 1986; 134:175-6). These results suggest that repetitive allergen exposure establishes a biochemical mechanism that sustains asthma in the absence of the inciting allergen. Tremendous progress has been made in uncovering the mechanisms surrounding the inception and development of asthma through animal model studies (Kips J C, et al. Murine models of asthma. Eur Respir J 2003; 22:374-82; Kumar R K, and Foster P S. Modeling allergic asthma in mice: pitfalls and opportunities. Am J Respir Cell Mol Biol 2002; 27:267-72). However, very little is known about the mechanisms regulating the persistence of chronic asthma.

In the majority of mouse models, asthma resolves spontaneously in 1 to 2 weeks (Duez C, et al. Fas deficiency delays the resolution of airway hyperresponsiveness after allergen sensitization and challenge. J Allergy Clin Immunol 2001; 108:547-56; Haworth O, et al. NK cells are effectors for resolvin E1 in the timely resolution of allergic airway inflammation. J Immunol 2011; 186:6129-35; Leech M D, et al. Resolution of Der p1-induced allergic airway inflammation is dependent on CD41CD251Foxp31 regulatory cells. J Immunol 2007; 179:7050-8). Repetitive allergen exposure in alternative models induces tolerance (Duez C, et al. Fas deficiency delays the resolution of airway hyperresponsiveness after allergen sensitization and challenge. J Allergy Clin Immunol 2001; 108:547-56; Kumar R K, et al. Reversibility of airway inflammation and remodeling following cessation of antigenic challenge in a model of chronic asthma. Clin Exp Allergy 2004; 34:1796-802; Schramm C M, et al. Chronic inhaled ovalbumin exposure induces antigen-dependent but not antigen-specific inhalational tolerance in a murine model of allergic airway disease. Am J Pathol 2004; 164:295-304). In others, cessation of repetitive allergen exposure results in resolution of inflammation (Chen Z G, et al. Neutralization of TSLP inhibits airway remodeling in a murine model of allergic asthma induced by chronic exposure to house dust mite. PLoS One 2013; 8:e51268; Henderson W R Jr, et al. A role for cysteinyl leukotrienes in airway remodeling in a mouse asthma model. Am J Respir Crit Care Med 2002; 165:108-16; Johnson J R, Wiley, et al. Continuous exposure to house dust mite elicits chronic airway inflammation and structural remodeling. Am J Respir Crit Care Med 2004; 69:378-85). The longest study period to date (Johnson J R, Wiley, et al.) has demonstrated attenuated airway remodeling and airway hyperreactivity persisting for 9 weeks after cessation of repetitive dust mite antigen exposure.

Recent studies have identified a novel IL-5/IL-13-producing type 2 innate lymphoid cell (ILC2) population in mice (Moro K, et al. Innate production of T(H)2 cytokines by adipose tissue-associated c-Kit(1)Sca-1(1) lymphoid cells. Nature 2010; 463:540-4; Neill D R, et al. Nuocytes represent a new innate effector leukocyte that mediates type-2 immunity. Nature 2010; 464:1367-70; Saenz S A, et al. IL25 elicits a multipotent progenitor cell population that promotes T(H)2 cytokine responses. Nature 2010; 464:1362-6) and human subjects (Mj€osberg J M, et al. Human IL-25- and IL-33-responsive type 2 innate lymphoid cells are defined by expression of CRTH2 and CD161. Nat Immunol 2011; 12:1055-62; Spits H, and Di Santo J P. The expanding family of innate lymphoid cells: regulators and effectors of immunity and tissue remodeling. Nat Immunol 2011; 12:21-7). Although numerous studies have established the importance of ILC2s in the initiation of airway eosinophilic inflammation, (Barlow J L, et al. IL-33 is more potent than IL-25 in provoking IL-13-producing nuocytes (type 2 innate lymphoid cells) and airway contraction. J Allergy Clin Immunol 2013; 132:933-41; Bartemes K R, et al. IL-33-responsive lineage-CD251 CD44(hi) lymphoid cells mediate innate type 2 immunity and allergic inflammation in the lungs. J Immunol 2012; 88:1503-13; Doherty T A, et al. STAT6 regulates natural helper cell proliferation during lung inflammation initiated by Alternaria. Am J Physiol Lung Cell Mol Physiol 2012; 303:L577-88; Halim T Y, et al. Lung natural helper cells are a critical source of Th2 cell-type cytokines in protease allergen-induced airway inflammation. Immunity 2012; 36:451-63; Kim H Y, et al. Innate lymphoid cells responding to IL-33 mediate airway hyperreactivity independently of adaptive immunity. J Allergy Clin Immunol 2012; 129:216-27, e211-6; Petersen B C, et al. Interleukin-25 induces type 2 cytokine production in a steroid-resistant interleukin-17RB1 myeloid population that exacerbates asthmatic pathology. Nat Med 2012; 18:751-8) the role of ILC2s in maintenance of existing airway hyperreactivity has not been established, and their involvement in the airways of asthmatic patients has not been examined.

### SUMMARY OF THE INVENTION

One embodiment of the present invention is method of treating severe asthma in a subject comprising obtaining a sample from the subject; detecting the presence of dual T helper 2/T helper 17 (Th2/Th17) cells in the sample from the subject; determining the frequency of the dual Th2/Th17 cells in the sample from the subject; determining the frequency of total cluster differentiation 4 (CD4) T helper cells in the sample from the subject; comparing the frequency of the dual Th2/Th17 cells to the frequency of the total CD4 T helper cells in the sample, wherein a frequency of greater than 5% of the dual Th2/Th17 cells as compared to the frequency of the total CD4 T helper cells further comprises detecting the presence of Th2 cells in the sample, determining the frequency of the detected Th2 cells in the sample and further comparing the frequency of the dual Th2/Th17 cells to the frequency of the Th2 cells, wherein a higher frequency of Th2/Th17 cells as compared to the frequency of the Th2 cells identifies the subject as having severe asthma; and administering to the subject identified as having severe asthma a compound selected from the group consisting of a bronchodilator, corticosteroid, leukotriene antagonist, anti-cytokine antibody, anti-cytokine receptor antibody, anti-IgE antibody, an antibiotic, a phosphodiesaterease inhibitor, an anti-MEK compound and combinations thereof for treating the subject.

In one aspect of the invention, the detecting the dual Th2/Th17 cells in the sample comprises detecting expression of CD4 (CD: cluster of differentiation antigen), CRTH2 (chemoattractant receptor-homologous molecule expressed on Th2 cells, also known as CD294, and G protein coupled receptor 44) and CCR6 (CC chemokine receptor 6), wherein co-expression of CD4, CRTH2 and CCR6 indicates the presence of the dual Th2/Th17 cells.

In still another aspect of the invention, detecting the presence of the dual Th2/Th17 cells comprises detecting expression of CD4, IL4 and IL17, wherein co-expression of CD4, interleukin-4 (IL4) and interleukin-17 (IL17) indicates the presence of dual Th2/Th17 cells.

In yet another aspect of the invention, detecting the presence of the dual Th2/Th17 cells comprises detecting expression of interleukin-1b (IL1b), wherein expression of IL1b indicates the presence of dual Th2/Th17 cells.

In another aspect of the invention, detecting the dual Th2/Th17 cells comprises determining the expression level of the complement factor C3 and/or C3a in the sample from the subject, wherein an elevated expression level of C3 or C3a as compared to the expression level of C3 and/or C3a from a healthy control, indicates the presence of dual Th2/Th17 cells.

In still another aspect of the invention, detecting the Th2 cells in the sample comprises detecting expression of CRTH2 on CD4 T cells in the sample, wherein expression of CRTH2 indicates the presence of the Th2 cells.

In yet another aspect of the invention, the step of determining the frequency of the dual Th2/Th17 cells, the CD4 T cells and the Th2 cells in the sample is flow cytometry.

In another aspect of the invention, a ratio of dual Th2/Th17 cells to Th2 cells of greater than 1 indicates a higher level of dual Th2/Th17 cells as compared to the level of Th2 cells.

Yet another embodiment of the present invention is a method of treating a subject having persistent asthma comprising obtaining a sample from the subject; determining the frequency of Type-2 cytokine-producing innate lymphoid (ILC2) cells in the sample, comparing the frequency of ILC2 cells from the subject to a control level, wherein an increased frequency of ILC2 from the subject as compared to the control identifies the subject as having persistent asthma; and administering to the subject a compound selected from the group consisting of a bronchodilator, corticosteroid, leukotriene antagonist, anti-cytokine antibody, anti-cytokine receptor antibody, anti-IgE antibody, an antibiotic, a phosphodiesaterease inhibitor, an anti-MEK compound and combinations thereof for treating the subject. In one aspect, the method further comprises determining the expression level of interleukin-33 (IL33) in the sample from the subject, wherein an increased level of IL33 as compared to the IL33 expression level from the control indicates the greater severity of the persistent asthma.

In one aspect of the invention, the greater the increase in the frequency of ILC2 cells as compared to the control indicates greater severity of the persistent asthma.

In still another aspect of the invention, the frequency of the ILC2 cells is determined by flow cytometry.

In yet another aspect, the expression level of IL33 is determined by ELISA.

Another embodiment of the present invention is a method of treating a subject having steroid resistant asthma comprising obtaining a sample from the subject; determining the expression level of mitogen-activated protein kinase (MEK) in the sample, comparing the expression level of MEK from the subject to an expression level of MEK from a control, wherein an increased expression level of MEK from the subject as compared to the control level identifies the subject as having steroid resistant asthma; and administering to the subject an anti-MEK compound or a non-steroid compound for treating the subject.

In one aspect, the greater the increase in the expression level of MEK from the subject as compared to the control level indicates greater severity of steroid resistance.

In yet another aspect, the expression level of MEK is determined by flow cytometry or ELISA.

In any of the embodiments of the invention described herein, the fluid is selected from bronchoalveolar lavage fluid (BAL), peripheral blood, nasal washing and induced sputum.

In one aspect of the invention a kit for determining the expression level of one or more genes selected from the group consisting of CD4, CRTH2, CCR6, IL-4, IL-17, IL1b, C3, C3a, ILC2, IL33, and MEK, wherein the kit comprises a component selected from the group consisting of an antibody, an antisense RNA molecule, and a molecular probe, and a molecular tag, wherein in the component detects the expression of the one or more genes.

### BRIEF DESCRIPTION OF THE DRAWINGS

FIGS. 1A-1H show the expression of Th2, Th17, and Th2/Th17 cells in BAL fluid. FIG. 1A-1C, show representative flow cytograms from a Th2-dominant asthmatic patient. BAL cells from an asthmatic patient were gated as shown in FIG. 1A, and then analyzed for CD4 T cells and CD1631 macrophages (FIG. 1A). The CD41CD1632 cell population was then analyzed for expression of IL-17 and IL-4 (FIG. 1B) or IL-5 (FIG. 1C). FIGS. 1D and 1E, show representative flow cytograms from a Th17^{predominant} (patient with recurrent pulmonary aspiration) and THh2/Th17^{predominant} (asthma) patient. BAL cells were processed as per FIG. 1A through 1C, and then analyzed for IL-4 and IL-17. FIG. 1H shows a heat map of the frequency of IL-41, IL-171, and dual-positive IL-4/IL-17 CD4 T cells in BAL fluid from 52 asthmatic patients. Each row is a single BAL sample. The embedded number indicates the actual frequency of cells. Based on the dominance of the cell type, asthmatic patients can be divided into TH2^{predominant}, Th2/Th17^{predominant}, and Th2/Th17^{low}. FIGS. 1F and 1G show expression of IL-4 and IL-17 (FIG. 1F) and coexpression of GATA3 and RORgt (FIG. 1G) in BAL cells by means of immunofluorescence staining. Cytospin preparations of BAL cells were double stained for IL-17 or GATA3 (green) and IL-4 or RORgt (red) and then counterstained with DAPI (blue) for nuclear staining. For GATA3/RORgt, Z-series images of a single lymphocyte were captured by using a confocal microscope. The images from a midsection show coexpression of GATA3 and RORgt in the nucleus of a single cell. Representative images from 3 separate experiments done with BAL cells from 3 different donors are shown.

FIGS. 2A-2E show BAL Th2/Th17 cells and clinical correlations. FIG. 2A shows the comparison of BAL dual-positive Th2/Th17 cells between asthmatic patients and disease control subjects (see the example section for description of disease control subjects). FIG. 2B shows the correlation between Th2/Th17 and Th2 cells in BAL from the Th2/Th17^{predominant} subgroup. FIG. 2C shows the correlation of BAL Th2, Th17, and Th2/Th17 cells and clinical parameters. Eos, Eosinophils; Lymph, lymphocytes. FIG. 2D shows the comparison of BAL IL-17A levels between asthmatic patients and disease control subjects. FIG. 2E shows the correlation between BAL IL-17A levels and FEV1.

FIGS. 3A-3D show the effect of dexamethasone on BAL cell expression of MKP1. BAL cells were cultured overnight (16 hours) with medium alone or dexamethasone (Dex; 10⁻⁷ Mol/L). The expression of MKP1 in CD4^{high} and CD4^{low} T cells was assessed by using flow cytometry (3A and 3B) and quantified (3C and 3D).

FIGS. 4A-4F show the effect of dexamethasone on BAL single-positive IL-4 and dual-positive IL-4/IL-17 CD4 T cells. BAL cells were cultured with medium or dexamethasone as in FIG. 3 and then analyzed for expression of IL-4 and IL-17 by means of flow cytometry as per FIG. 1. FIGS. 4A-B and D-E are representative flow cytograms from 2 patients, one with dominant IL-4+ cells (FIGS. 4A and 4B) and the other with both IL-4 and dual-positive IL-4/IL-17 cells (FIGS. 4D and 4E), are shown. FIGS. 4C and 4F, show the effect of dexamethasone (Dex) on Th2 and Th2/Th17 cells from 14 asthmatic patients. The numbers on the top of the graphs represent statistical significance.

FIGS. 5A-5H show the preferential expression of dual-positive TH2/TH17 cells in the BAL MEK^{high} CD4 T-cell population. FIGS. 5A and 5B, show the sensitivity of MEK^{high} and MEK^{low} CD4 populations to dexamethasone-induced cell death. Data are presented as percentages and absolute cell counts (in parentheses) per boxed area. FIG. 5C shows the gating strategy for BAL CD4 T cells. FIG. 5D shows the gating strategy for separation of MEK^{high} and MEK^{low} cells in CD4-gated cells. FIGS. 5E and 5F show expression of dual-positive TH2/TH17 cells in MEK^{high} and MEK^{low} BAL CD4 T-cell populations. FIGS. 5G and 5H show the comparison of expression of dual-positive IL-4/IL-17 (FIG. 5G) and pSTAT3/pSTAT6 (FIG. 5H) cells in MEK^{high} and MEK^{low} cell populations.

FIGS. 6A-6F show TH2^{predominant}, TH2/TH17^{predominant}, and TH2/TH17^{low} subgroups of asthmatic patients and their clinical features. FIGS. 6A-6C show all asthmatic patients (n=52) were grouped based on the dominant expression of single positive IL-4 cells (TH2^{predominant}), dual-positive IL-4/IL-17 cells (TH2/TH17^{predominant}), or 5% or less of either cell types (TH2/TH17^{low}). FIGS. 6D-6F, show the comparison of PC20 for methacholine, FEV1, and blood eosinophilia among the 3 subgroups.

FIG. 7A shows the forward scatter (FS) and side scatter (SS) display of BAL cells presented and analyzed in FIG. 1A through 1C. The boxed area shows the gating strategy for subsequent analysis.

FIG. 7B shows the correlation of IL-4 and IL-5 expression by BAL CD4 T cells, as presented in FIGS. 1B and 1C.

FIGS. 8A-8D are flow cytograms of BAL cells stained with single (FIG. 8A-8C) and double (FIG. 8D) isotype control antibodies.

FIG. 9A is a forward scatter (FS) and side scatter (SS) display of BAL cells from a different asthmatic patient. The boxed area is gated for further analysis.

FIG. 9B, shows gated cells were analyzed for expression of CD4 (T-cell marker) and CD68 (macrophage marker).

FIG. 9C, shows the same gated cells as in FIG. 9A, were also analyzed for CD3ε and CD4. CD3+CD4+(FIG. 9D) and CD3+CD4−(FIG. 9E) T cells from the flow cytogram in FIG. 9C, were then analyzed for expression of IL-4 and IL-17. Representative flow cytograms from one of 4 different study subjects are shown.

FIGS. 10A-10F shows the staining patterns of IL-4, IL-17, and IL-4/IL-17 in BAL CD4 T cells from select Th2^{predominant} (FIGS. 10A and 10B), Th2/Th17^{predominant} (FIGS. 10C and 10D), and TH17^{predominant} (FIGS. 10E and 10F) BAL donors. Flow cytograms presented in this figure are from asthmatic patients, and that in FIG. 10F, is from a patient with chronic pulmonary aspiration.

FIG. 11A shows the confocal Z-series images of a BAL lymphocyte immunofluorescently stained with DAPI and antibodies against GATA3 and RORgt. The bottom panel shows an overlay of the images.

FIG. 11B shows images of BAL lymphocytes immunofluorescently stained with DAPI and antibodies against GATA3 and RORgt from 4 different donors.

FIGS. 12A-12C shows the coexpression of pSTAT3 and pSTAT6 in CD4 T cells from asthmatic patients. FIGS. 12A and 12B shows BAL cells were gated for CD4 T cells and then examined for coexpression of IL-4 and IL-17 (FIG. 12A) and pSTAT3 and pSTAT6 (FIG. 12B).

FIG. 12C shows the correlation between pSTAT3 and pSTAT6 expression in 9 asthmatic patients.

FIGS. 13A-13C shows the coexpression of CCR6 and CRTH2 in BAL CD4 T cells. FIGS. 13A and 13B show BAL cells were immunostained for CD4, CCR6, CRTH2, IL-4, and IL-17. CD4 T cells were gated and analyzed for coexpression of CCR6 and CRTH2 or IL-4 and IL-17. FIG. 13C, shows the correlation of CCR6/CRTH21 and IL-4/IL-171 cells from 6 donors.

FIGS. 14A-14C shows the persistence of an asthma phenotype in a mouse model. FIG. 14A shows airway hyperreactivity (total lung resistance [RL]) measured 1, 2, and 6 months after cessation of allergen exposure. FIG. 14B shows the corresponding histologic images of the lung from mice of the chronic asthma and saline control groups. Saline control was from the 6-month time point. Scale bar 5 100 mm. FIG. 14C, shows the quantification of peribronchial and perivascular inflammation as a percentage total area in the field. There were 5 to 8 mice per group. ***P<0.001.

FIGS. 15A-15F shows chronic asthma persists after T-cell depletion. FIG. 15A, shows the timeline of intranasal allergen exposure for 6 weeks, the rest period, and then irradiation, bone marrow transfer, and antibody and inhibitor treatment in weeks 9 and 10 in the chronic asthma model. Outcomes were measured 3 days later, unless otherwise stated. FIG. 15B, shows the effect of an anti-CD3ε or isotype control antibody on airway resistance (n=5). FIG. 15C, shows the effect of adoptive transfer of spleen CD4 T cells from the chronic asthma and saline control groups obtained in week 10 to naive mice. Airway hyperreactivity was measured in chronic asthma CD4 recipient mice on days 6 and 21 and in saline control CD4 recipient mice on day 6 (n=4). *P<0.05. RL, Total lung resistance. FIG. 15D shows the quantification of lung inflammation at weeks 11, 13, and 15 in mice after lethal irradiation and bone marrow transplantation. FIG. 15E, shows the quantification of epithelial hypertrophy and peribronchial smooth muscle hypertrophy at week 15. All morphometric quantifications were performed in at least 5 independent fields per mouse. BM, Basement membrane. FIG. 15F, shows the measurement of lung resistance in week 15. *P<0.05, **P<0.01, ***P<0.001, and #P<0.05. n.s., Not significant. There were 5 to 6 mice per group for FIG. 15D through 15F.

FIGS. 16A-16K show the comparison of the gene expression profile between chronic and acute asthma by using a microarray. FIG. 16A, Microarray analysis of lung tissue obtained from mice with chronic and acute asthma (n=3 mice per group). The table shows differentially expressed genes in the asthmatic groups in comparison with the saline control group. FIG. 16B, Principal component analysis (PCA) of the expressed genes demonstrating differences among the asthma models, as reflected by their spatial positioning in the 3-dimensional space. FIG. 16C, Heat map of select gene expression changes in the chronic and acute asthma compared with saline control groups. FIGS. 16D-I, Measurement of mRNA for select genes by using real-time PCR. The mRNA level was normalized to glyceraldehyde-3-phosphate dehydrogenase (GAPDH) mRNA expression (n=5 mice per group). *P<0.05 compared to saline control; ̂P<0.05 compared to acute asthma. FIG. 16J, IL-33 levels were confirmed by means of Western blotting to determine the presence of both full-length IL-33 and the mature cleaved product (n=3). FIG. 16K, Comparison of lung IL-5+CD3 T cells and IL-5+ ILC2s among the study groups (n=5). *P<0.05 compared with CD3+IL-5+ cells in each study group.

FIGS. 17A-17G show the role of ILCs in the persistence of asthma. FIG. 17A shows the quantification of the lung ILC population in the chronic asthma group and in the chronic asthma and saline control groups after immune ablation and bone marrow transplantation. FIGS. 17B and 17C show the total lung cytokine-producing cell populations and cytokine-positive host CD45.2+ and transplanted donor CD45.1+ ILC populations (mean±SEM) were quantified for IL-5 (FIG. 17B) and IL-13 (FIG. 17C; n=5 mice per group). *P<0.05. FIG. 17D, Bone marrow from either Rag1−/−, Rag2−/−:γc−/−, or wild-type mice was transplanted into mice with chronic asthma after irradiation. Airway hyperreactivity was measured 6 weeks after irradiation. FIGS. 17E and 17F, Effect of IL-33 blockade after immune ablation and bone marrow transplantation: total IL-13+ lung cells and IL-13+ ILC2s (FIG. 17E) and airway hyperreactivity (FIG. 17F). RL, Total lung resistance. *P<0.05, **P<0.01, ***P<0.001, ##P<0.05, and ###P<0.01 in anti-IL-33 vs IgG. There were 6 to 9 mice per group. Results shown are means±SEMs. FIG. 17G, Effect of adoptive transfer of ILCs from mice with chronic asthma and those in the saline control group to naive mice on airway hyperreactivity measured 21 days later (n=5). *P<0.05.

FIGS. 18A-18J show the role of IL-33 in the persistence of asthma. FIG. 18A, A549 cells were cultured in medium alone or with IL-33 (10 ng/mL), IL-1b (2 ng/mL), IL-4 (10 ng/mL), IL-13 (20 ng/mL), IL-17 (10 ng/mL), TNF (2 ng/mL), IFN-γ (10 ng/mL), or IFN-γ plus TNF (same concentrations) for 72 hours, and then mRNA was analyzed for IL-33 normalized to glyceraldehyde-3-phosphate dehydrogenase (GAPDH). *P<0.05 and **P<0.01. There were 4 to 8 mice per group. FIGS. 18B-18E show the direct effect of IL-33 on airway hyperreactivity, lung ILCs, and autoinduction. Mice were treated intranasally for 3 consecutive days with 400 ng per dose of IL-33: methacholine-induced airway hyperreactivity (total lung resistance [RL]; FIG. 18B), lung IL-13+ ILCs (FIG. 18C), and IL-33 autoinduction (fluorescence intensity [gray, goat IgG control; FIG. 18D]; IL-33+ cells [FIG. 18E]) were measured after 15 days (n=5 mice per group). *P<0.05, **P<0.01, and ***P<0.001. IN, Intranasal. FIG. 18F shows the cytokine induction of IL-33R mRNA in A549 cells (n=4 per group). *P<0.05 and **P<0.01. FIG. 18G shows A549 cells were pretreated for 24 hours with either medium or the cytokines, as in FIG. 18A, and then treated for 72 hours with or without IL-33 (20 ng/mL; n=4). **P<0.01. FIGS. 18H-18I shows the persistence of IL-33+ cells (fluorescence intensity in FIG. 18H, and IL-33+ cell numbers in FIG. 18I) in mice with chronic asthma after irradiation and transfer of bone marrow from naive, Rag1−/−, and Rag2−/−: γc−/− mice (n=5 per group). *P<0.05. n.s., Not significant. FIG. 18J shows the effect of 3 doses of an anti-IL-13 antibody on total lung resistance (RL). The change in RL with the 25-mg dose of methacholine is shown (n=5 per group). *P<0.05.

FIGS. 19A-19F show the role of IL-33 and ILC2s in human asthma. FIG. 19A shows IL-33 levels (ELISA) in BAL fluid from asthmatic patients and disease control subjects. **P<0.005. FIG. 19B shows the correlation between BAL fluid IL-33 levels and FEV1, a measure of airway obstruction. FIG. 19C shows IL-33 immunostaining of an endobronchial biopsy sample from an asthmatic patient (n 5 9). Green, IL-33; blue, 49-6-diamidino-2-phenylindole dihydrochloride (DAPI) for nuclear staining. FIGS. 19D and 19E show the frequency of ILC2s (lin-FcεRI-IL-7Rc+IL-33R+) and IL-13+ ILC2s in BAL fluid from asthmatic patients and disease control subjects. FIG. 19F shows the summary diagram depicting the mechanism of sustained IL-33 production and persistence of asthma. Repetitive allergen exposure induces IL-33 production by epithelial cells. IL-33 induces ILC2s and TH2 cells and stimulates IL-13 production. IL-13 directly induces IL-33 production, which establishes feedback circuit 1. IL-33 autoinduces, which establishes feedback circuit 2. IL-13 augments IL-33R expression and enhances IL-33 autoinduction, thus generating a feed-forward circuit.

FIGS. 20A-20D shows the expression of mucin (red) expression and toluidine-positive (dark brown) mast cells in airways of mice from the chronic asthma and saline control groups.

FIGS. 21A and 21B show the effect of an anti-CD3ε or an isotype control antibody on spleen TCR-β+ T cells (FIG. 21A) and airway inflammation (FIG. 21B). Three doses (200 mg per dose) of the antibodies were administered on alternate days at week 10 (n=5). *P=0.02.

FIGS. 21C-21E show T-cell proliferation and cytokine production 6 weeks after irradiation at week 9. Spleen (sp) and mediastinal lymph node (medLN) CD4+ T cells were obtained from 3 study groups: chronic asthma (CA), chronic asthma plus ablation (CA+A), and saline control plus ablation (S+A). T cells (2×10⁶ cells per well) were stimulated with medium, dust mite (10 μg/mL), and anti-CD3 (1 μg/mL) plus anti-CD28 (1 μg/mL) antibodies for 96 hours. Proliferation was assessed by means of flow cytometric analysis of carboxyfluorescein succinimidyl ester (CFSE) dilution. For flow cytometric detection of intracellular IL-2 (FIG. 21D) and IL-4 (FIG. 21E), cells were treated with monensin 6 hours before conclusion of the culture. Twenty thousand events (live cells) per well were sampled for flow cytometric analyses (n=4). *P<0.05 compared with medium control.

FIG. 21F shows the effect of irradiation and immune ablation on lung inflammation. Representative histologic images of the lung sections from the study mice are as shown in FIG. 15D. Scale bar 5 100 mm.

FIGS. 22A-22K show the characterization of lung ILCs. FIGS. 22A and 22B show collagenase-digested single lung cells were gated on live cells through forward scatter/side scatter and then selected for the hematopoietic CD45+ population. The ILCs were identified as a lineage (CD3, Ly-6G/Ly-6C, CD11b, CD45R/B220, TER-119/erythroid cells)-negative and CD25+ population. FIGS. 22C-22H show this subpopulation was positive for ILC2 markers, including c-kit, Sca-1, intracellular IL-13 and IL-5, and cell-surface CRTH2, IL-33R, and killer cell lectin-like receptor G superfamily, member 1 (KLRG1) but negative for the natural killer cell marker NK1.1 or mast cell/basophil marker FcεRI. FIG. 22I shows the total IL-17+ lung cells in the chronic asthma and saline control groups, as detected by using flow cytometry. Lung ILCs (lin-CD25+) were negative for IL-17 (n=3). FIGS. 22J and 22K show the comparison of ILCs between the chronic asthma and chronic asthma after immune ablation groups.

FIGS. 23A-23E show the effect of irradiation on ILCs. A and B, Frequency of ILCs (lin-CD25+CD45+ cells) in the lung digest from mice treated with saline alone (control for chronic asthma) and saline followed by irradiation and transplantation of bone marrow from a naive mouse. Measurements were done 6 weeks after bone marrow transplantation. FIG. 23C, Comparison of total lung ILC numbers between the 2 groups. FIG. 23D shows the comparison of total lung IL-5+ cell numbers and host- and donor-derived IL-5+ ILC2 numbers. FIG. 23E shows the comparison of total lung IL-13+ cell numbers and host- and donor-derived IL13+ ILC2 numbers. There were 5 mice per group for FIG. 23C through 23E. n.s., Not significant.

FIGS. 24A-24E show the effect of marrow transplantation on the persistence of inflammation. FIGS. 24A and 24B show that chronic asthma was induced as per FIG. 15A, and bone marrow from either Rag1−/− or Rag2−/−: γc−/− mice was transferred to mice after irradiation. Outcomes were measured 6 weeks after irradiation at week 15. FIG. 24A, Differential cell counts in BAL fluid. FIG. 24B, Representative lung sections stained with H&E from the mice represented in FIG. 24A, Scale bar is 100 μm. FIG. 24C, Peribronchial and perivascular inflammation as a percentage of total area. FIGS. 24D and 24E, Flow cytometric analysis of lung digests from Rag1−/− and Rag2−/−: γc−/− bone marrow recipient mice showing changes in total ILC (FIG. 24D) and IL-13+ ILC (FIG. 24E) numbers. *P<0.05 and **P<0.01. There were 5 mice per group.

FIGS. 25A and 25B show the effect of anti-IL-33 on the persistence of airway inflammation (n=4). FIG. 25A, Representative lung sections stained with H&E. Scale bar is 100 μm. FIG. 25B, Differential cell counts in BAL fluid.

FIG. 25C, Effect of intranasal anti-IL-33 antibody on airway inflammation. Representative lung sections were stained with H&E. Scale bar is 100 μm. *P<0.05, **P<0.01, and ***P<0.001.

FIGS. 26A-26B shows the effect of adoptive transfer of ILCs. CD45-lin-CD25+ cells from the lung digest obtained from the chronic asthma and saline control models were adoptively transferred to naive mice. The presence of host- and donor-derived total ILCs (FIG. 26A) and IL-13+ ILC2s (FIG. 26B) in the lungs was analyzed on day 21 after transfer (n=5 per group). *P<0.05.

FIG. 27A, Effect of IL-13 on IL-33 secretion. A549 cells were treated overnight with medium or IL-13 (20 ng/mL) and ATP (100 mmol/L), and the released IL-33 was measured by using ELISA (n=6). *P=0.04. FIGS. 27B-27E, Direct effect of IL-33 on airway hyperreactivity and lung ILCs. C57Bl/6 mice were treated intranasally for 3 consecutive days with 400 ng per dose of IL-33. Outcome measures were evaluated 15 days after final exposure.

FIGS. 27B and 27C, Airway inflammation and its quantification as a percentage of total area.

FIGS. 27D and 27E, Effect on lung ILCs: flow cytometric analysis of lung digests showing total ILCs (FIG. 27D) and total IL-5+ lung cells and IL-5+ ILC2s (FIG. 27E). *P<0.05, **P<0.01, and ***P<0.001. There were 5 mice per group.

FIG. 28A shows lung digest from the chronic asthma model was stained for IL-33 and pro-surfactant protein C(Pro-SPC), CD11b, or FcεRI (n=5).

FIG. 28B, A549 cells were treated overnight with DRA, and IL-33 mRNA was quantified by using real-time PCR. *P=0.04. There were 5 mice per group.

FIGS. 29A-29B show the isolation strategy of ILC2s from human BAL fluid. A, BAL cells were gated for lin− (CD3, CD14, CD16, CD19, CD20, CD56)-negative and FcεRI− cells. These lin-FcεRI− cells were stained for IL-7Ra and IL-33R (also known as ST2: suppressor of tumorigenicity). The IL-7Rc+IL-33R+ cells were identified as ILC2s. The majority of these cells were positive for IL-5 and IL-13. They were partially positive for CD161 (FIG. 29B). The gating strategy and threshold quadrants were based on isotype antibody controls (data not shown).

### DETAILED DESCRIPTION OF THE INVENTION

This invention generally relates to improved methods and kits for identifying/predicting and/or treating severe asthma and/or persistent asthma in subjects by analyzing for the presence of certain cells and/or by determining the expression of one or more genes as disclosed herein. The inventors have made the surprising finding that asthma is associated with a higher frequency of dual-positive Th2/Th17 cells in BAL fluid and that the Th2/Th17^{predominant} subgroup of asthmatic patients manifests steroid (such as glucocorticoids) resistance in vitro. This subgroup was also determined to have the greatest airway obstruction and hyperreactivity compared with Th2^{predominant} and Th2/Th17^{low} subgroups. Th2 cells (also referred to as TH2) and Th2/Th17 cells (also referred to as dual Th2/Th17 cells or TH2/TH17 cells) are subgroups of CD4 T helper cells. Asthma is hererogenous, thus some subjects have only Th2 cells, others have both Th2 and Th2/Th17, and yet others have no Th2 or Th2/Th17 cells. Those who have both Th2 and Th2/Th17 cells may fall into categories or subgroups (or endotypes): Th2-predominant (Th2^{predominant}) and Th2/Th17-predominant (Th2/Th17^{predominant}). The inventors have made the surprising finding that of these subjects, the Th2/Th17^{predominant} subgroup have been found to have more severe asthma, thus the Th2/Th17^{predominant} asthma is more severe than Th2^{predominant} asthma and a further subgroup of Th2/Th17^{low}. A predominant subgroup is defined by having a higher amount or frequency of a specific subgroup of cells (Th2, Th2/Th17 cells) compared to the amounts of one or more other subgroups. For example, a Th2/Th17^{predominant} subgroup is a subgroup in which the subject(s) has been determined to have a higher amount or frequency of dual Th2/Th17 cells as compared to the amount of Th2 cells. A Th2^{predominant} subgroup is a subgroup in which the subject(s) has been determined to have higher amount or frequency of Th2 cells as compared to the amount of dual Th2/Th17 cells. A predominant subgroup has greater than 5% of the total T cells belonging to that specific subgroup. A low subgroup is defined by having 5% or less of total T cells belonging to that subgroup. For example, a Th2/Th17^{low} subgroup is a subgroup in which the subject(s) has been determined to have a frequency of Th2/Th17 cells that is 5% or less than the total CD4 T helper cells.

In addition, the inventors have found that elimination of T cells though antibody-mediated depletion or lethal irradiation and transplantation of recombination-activating gene (Rag1)−/− bone marrow in mice with chronic asthma resulted in resolution of airway inflammation but not airway hyperreactivity or remodeling. Elimination of T cells and type 2 innate lymphoid cells (ILC2s) through lethal irradiation and transplantation of Rag2−/−: γc−/− bone marrow or blockade of interleukine-33 (IL-33) resulted in resolution of airway inflammation and hyperreactivity. Persistence of asthma was found to require multiple interconnected feedback and feed-forward circuits between ILC2s and epithelial cells. Additionally, epithelial IL-33 was found to induce ILC2s, a rich source of IL-13. The latter directly induced epithelial IL-33, establishing a positive feedback circuit. IL-33 autoinduced, generating another feedback circuit. IL-13 upregulated IL-33 receptors and facilitated IL-33 autoinduction, thus establishing a feed-forward circuit. Elimination of any component of these circuits resulted in resolution of chronic asthma. In agreement with the foregoing, IL-33 and ILC2 levels were increased in the airways of asthmatic patients. In addition, IL-33 levels correlated with disease severity. The inventors describe a critical network of feedback and feed-forward interactions between epithelial cells and ILC2s involved in maintaining chronic asthma. Although T cells contributed to the severity of chronic asthma, they were redundant in maintaining airway hyperreactivity and remodeling.

The present invention provides for a method of identifying and/or treating a subject having severe asthma by detecting the presence of Th2/Th17 cells as well as detecting the presence of Th2 cells in a sample from the subject and determining and measuring and comparing the level of the Th2/Th17 cells to the level of Th2 cells in the sample, wherein a higher level or frequency of Th2/Th17 cells as compared to the level of the Th2 cells in the biological sample from the subject, identifies the subject as having severe asthma. In one aspect, the subject identified as having severe asthma is then treated. As used herein severe asthma is defined as sustained xacerbation of asthma that has not been found to respond to standard treatments of asthma with bronchodilators (inhalers) and steroids.

The dual Th2/Th17 cells can be identified and/or detected by detecting the expression of a combination of genes. Such a combination of genes includes cluster of differentiation antigen 4 (CD4), chemoattractant receptor-homologous molecule expressed on Th2 cells (CRTH2; also known as CD294, and G protein coupled receptor 44) and CC chemokine receptor 6 (CCR6). In one aspect, co-expression of all three genes (CD4, CRTH2 and CCR6) indicates or serves as a surrogate for the presence of the dual Th2/Th17 cells. Another combination of genes includes CD4, IL4 and IL17, wherein detection of the co-expression of all three of these genes indicates the presence of Th2/Th17 cells. Once co-expression is identified or detected, the frequency of Th2/Th17 cells is determined. If the frequency of the Th2/Th17 cells is determined to be less than 5% of the total CD4 T cells in the sample, then this subgroup or endotype is a Th2/Th17^{low} subgroup. If however, the frequency of the Th2/Th17 cells is determined to be at least 5% or greater of the total CD4 T cells in the sample, then the frequency of Th2 cells is determined and a comparison is then performed between the frequency of the Th2/Th17 cells and the Th2 cells. If the frequency of the Th2/Th17 cells is higher compared to the frequency of the Th2 cells, this subgroup or endotype is a Th2/Th17^{predominant}. If however, the frequency of the Th2 cells is higher compared to the frequency of the Th2/Th17, then the subgroup is referred to as a Th2^{predominant} subgroup or endotype. When comparing the determined levels or frequency of the dual Th2/Th17 cells to the determined levels or frequency of the Th2 cells, a ratio of greater than 1 indicates a higher level or frequency of dual Th2/Th17 cells as compared to Th2 cells.

In addition, the inventors have found that the dual Th2/Th17 cells can be detected by determining the expression level of complement factor C3 and/or C3a. The inventors have determined that an elevated expression level of C3 and/or C3a in a subject as compared to the expression level of C3 and/or C3a from a healthy control indicates the presence of Th2/Th17 cells.

In order to detect and/or determine the level or frequency of Th2 cells in a sample, expression of CRTH2 on CD4 T cells in the sample is detected. If expression of CRTH2 is detected, then Th2 cells are present in the sample.

The level or frequency of dual Th2/Th17 cells and/or the level or frequency of Th2 cells and/or the total CD4 T cells in a sample can be determined and/or measured by methods such as flow cytometry, ELISA, real-time PCR or immunofluorescence/immunocytochemical staining or a combination thereof. In a preferred aspect, flow cytometry is used.

Another embodiment of the present invention is a method to identify and/or predict and/or treat a subject having persistent asthma by determining the frequency of ILC2 cells in a sample from the subject wherein an increased frequency of ILC2 cells from the subject as compared to a non-allergic healthy control identifies and/or predicts that the subject has persistent asthma. In one aspect the identified subject is then treated. The inventors have found that the greater the frequency of ILC2 cells from the subject as compared to the control indicates greater severity of the persistent asthma. In one aspect of this embodiment, the method further comprises determining the expression level of IL33 in the sample from the subject, wherein an increased level of IL33 (for example >490 pg/ml of bronchoalveolar lavage fluid) as compared to the level that is present in bronchoalveolar lavage from non-allergic healthy control subjects, indicates greater severity of the persistent asthma. In one aspect, the frequency of the ILC2 cells in the sample from the subject is determined by flow cytometry. In another aspect, the expression level of IL33 is the sample from the subject is determined by ELISA.

A further embodiment of the present invention is a method to identify and/or predict and/or treat a subject having steroid resistant asthma by determining the expression level of MEK ERK kinase 1 (MEK), wherein an increased expression level of MEK as compared to a non-allergic healthy control identifies and/or predicts that the subject has steroid resistant asthma. In one aspect the identified subject is then administered an anti-MEK compound or a non-steroid compound. The inventors have found that the greater the increase in the expression level of MEK as compared to the control MEK expression level indicates greater severity of steroid resistance. In one aspect, the expression level of MEK is determined by methods such as flow cytometry and/or ELISA.

In one aspect of the methods of the invention, the sample is from bronchoalveolar lavage fluid (BAL), peripheral blood (including serum), nasal washing or induced sputum.

A subject having persistent asthma means that the subject has asthma symptoms every 30 day. The subject may need to use a rescue inhaler daily and typically a subject's normal activities are affected by symptoms such as wheezing, shortness of breath and/or chest tightness. Persistent asthma can be mild, moderate or severe.

As used herein, the term “expression”, when used in connection with detecting the expression of a gene, can refer to detecting transcription of the gene (i.e., detecting mRNA levels) and/or to detecting translation of the gene (detecting the protein produced). To detect expression of a gene refers to the act of actively determining whether a gene is expressed or not. This can include determining whether the gene expression is upregulated as compared to a control, downregulated as compared to a control, or unchanged as compared to a control or increased or decreased as compared to a reference level. Therefore, the step of detecting expression does not require that expression of the gene actually is upregulated or downregulated or increased or decreased, but rather, can also include detecting that the expression of the gene has not changed (i.e., detecting no expression of the gene or no change in expression of the gene). In addition, the expression level of one or more genes disclosed herein that are strongly correlated with IL-13 can be differentially expressed.

In addition to the methods already disclosed, expression of transcripts and/or proteins can be measured by any of a variety of known methods in the art. For RNA expression, methods include but are not limited to: extraction of cellular mRNA and Northern blotting using labeled probes that hybridize to transcripts encoding all or part of the gene; amplification of mRNA using gene-specific primers, polymerase chain reaction (PCR), and reverse transcriptase-polymerase chain reaction (RT-PCR) and/or RNA Ampliseq, followed by quantitative detection of the product by any of a variety of means; multiplexed quantitative PCR enrichment of cDNA amplicons, followed by conversion of amplicons to sequence libraries and Next-generation based sequencing of libraries to generate digital count expression data; extraction of total RNA from the cells, which is then labeled and used to probe cDNAs or oligonucleotides encoding the gene on any of a variety of surfaces; in situ hybridization; and detection of a reporter gene.

Methods to measure protein expression levels generally include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry, as well as assays based on a property of the protein including but not limited to enzymatic activity or interaction with other protein partners. Binding assays are also well known in the art. For example, a BIAcore machine can be used to determine the binding constant of a complex between two proteins. The dissociation constant for the complex can be determined by monitoring changes in the refractive index with respect to time as buffer is passed over the chip (O'Shannessy et al., 1993, Anal. Biochem. 212:457; Schuster et al., 1993, Nature 365:343). Other suitable assays for measuring the binding of one protein to another include, for example, immunoassays such as enzyme linked immunoabsorbent assays (ELISA) and radioimmunoassays (RIA); or determination of binding by monitoring the change in the spectroscopic or optical properties of the proteins through fluorescence, UV absorption, circular dichroism, or nuclear magnetic resonance (NMR).

In addition, the presence of cells and/or the frequency of cells are detected. The frequency of the cells such as CD4 T cells and/or Th2 and/or Th2/T17 and/or ILC2 is determined by a method including by not limited to flow cytometery.

When comparing the expression level of any one or more genes as disclosed herein, it is to be understood that the expression level of the any one or more genes is compared with the same gene or genes from the reference or control. For example, if the expression level of CRTH2 and CCR6 are to be determined or analyzed, then the expression level of CRTH2, and CCR6 from the subject would be compared to the expression level of CRTH2, and CCR6 from a healthy control. The expression level of any one or more genes as disclosed herein is considered altered if the expression level of the one or more genes as compared to the expression level of the same one or more genes from the reference or healthy control is increased or decreased (upregulated or downregulated).

In one aspect, the expression levels of at least one, at least two, or at least three, of the genes are altered (i.e. increased, decreased or be a combination of expression levels) as compared to the corresponding genes in a control, wherein one or more of the gene expression levels can be increased (or the genes are upregulated) as compared to the control expression level. In one aspect, the gene expression level of the one or more genes is at least about a 2 fold, at least about a 3 fold, at least about a 4 fold, at least about a 5 fold, at least about a 10-fold, at least about a 20 fold, at least about a 25 fold, at least about a 30 fold, at least about a 40 fold or at least about a 50 fold difference from the expression level of the healthy control.

As used herein, reference to a control (or reference), means a subject (or group of subjects) who is a relevant control to the subject being evaluated by the methods of the present invention. The control can be matched in one or more characteristics to the subject. More particularly, the control can be matched in one or more of the following characteristics, gender, age, disease state, including a healthy control that has been determined to be disease-free. The control expression level used in the comparison of the methods of the present invention can be determined from one or more relevant control subjects. Additionally, a control can be a healthy individual or group of individuals that have been determined to not have an airway disease or condition such as asthma.

The invention also provides for treating the subject. In some aspects, the subjects can be treated by administration of one or more compounds including but not limited to bronchodilators (such as beta agonists and anti-cholinergics), corticosteroids, leukotriene antagonists, anti-cytokine antibodies, anti-cytokine receptor antibodies, anti-IgE antibody, antibiotics, a phosphodiesaterease inhibitor, an anti-MEK compound (such as Trametinib, Selumetinib and Cobimetinib) and combinations thereof.

The invention also provides for a kit for determining expression level of one or more genes described herein (CD4, CRTH2, CCR6, IL-4, IL-17, IL1b, C3, C3a, IL33, and MEK). The kit can comprise one or more components selected from an antibody, an antisense RNA molecule, and a molecular probe or tag, wherein in the component detects the expression and/or frequency of the one or more genes. The kit further comprises pharmaceutically acceptable carriers.

The presence of allergic sensitivity (skin test result positivity or IgE-specific antibody in serum) can be detected in a vast majority, but not all, asthmatic patients (Romanet-Manent S, et al. Allergic vs nonallergic asthma: what makes the difference? Allergy 2002; 57:607-13). In addition to IgE, mild-to moderate eosinophilia is a characteristic feature of asthma. Interestingly, some nonallergic asthmatic patients also have blood eosinophilia. Both IgE antibody and eosinophilia are driven by a Th2-type immune response. Thus the presence of Th2 cells in lung tissue and BAL fluid is anticipated and has previously been reported (Robinson D, et al. Activation of CD41 T cells, increased Th2-type cytokine mRNA expression, and eosinophil recruitment in bronchoalveolar lavage after allergen inhalation challenge in patients with atopic asthma. J Allergy Clin Immunol 1993; 92:313-24; Krug N, et al. Cytokine profile of bronchoalveolar lavage-derived CD4(1), CD8(1), and gammadelta T cells in people with asthma after segmental allergen challenge. Am J Respir Cell Mol Biol 2001; 25:125-31; Thunberg S, et al. Allergen provocation increases TH2-cytokines and FOXP3 expression in the asthmatic lung. Allergy 2010; 65:311-8; Walker C, et al. Activated T cells and cytokines in bronchoalveolar lavages from patients with various lung diseases associated with eosinophilia. Am J Respir Crit Care Med 1994; 150:1038-48; Leung D Y, et al. Dysregulation of interleukin 4, interleukin 5, and interferon gamma gene expression in steroidresistant asthma. J Exp Med 1995; 181:33-40; Huang S K, et al. IL-13 expression at the sites of allergen challenge in patients with asthma. J Immunol 1995; 155:2688-94; Brightling C E, et al. TH2 cytokine expression in bronchoalveolar lavage fluid T lymphocytes and bronchial submucosa is a feature of asthma and eosinophilic bronchitis. J Allergy Clin Immunol 2002; 110:899-905). The median frequency of BAL IL-4+CD4 T cells in asthmatic patients in published reports has ranged from 5% 40 to 9% (Krug N, et al. Cytokine profile of bronchoalveolar lavage-derived CD4(1), CD8(1), and gammadelta T cells in people with asthma after segmental allergen challenge. Am J Respir Cell Mol Biol 2001; 25:125-31). The inventors have observed a median frequency of 11% IL-4+CD4 T cells in BAL fluid from asthmatic patients. The relatively higher frequency could be due to the difference in severity of asthma and the number of patients studied. The previous studies were performed on a small number (n=11-12) of patients whose asthma was relatively mild (median forced expiratory volume (FEV1), 90% to 100%). The inventors studied 52 patients with severe asthma whose median FEV1 was 73.5%. Th2-type cytokines, especially IL-5 and IL-13, have been recovered from BAL fluid obtained from asthmatic patients, (Krug N, et al. Cytokine profile of bronchoalveolar lavage-derived CD4(1), CD8(1), and gammadelta T cells in people with asthma after segmental allergen challenge. Am J Respir Cell Mol Biol 2001; 25:125-31; Thunberg S, et al. Allergen provocation increases Th2-cytokines and FOXP3 expression in the asthmatic lung. Allergy 2010; 65:311-8) although some studies did not observe any increase in levels of these cytokines (Bossley C J, et al. Pediatric severe asthma is characterized by eosinophilia and remodeling without T(H)2 cytokines. J Allergy Clin Immunol 2012; 129:974-82.e13; Batra V, et al. Bronchoalveolar lavage fluid concentrations of transforming growth factor (TGF)-beta1, TGF-beta2, interleukin (IL)-4 and IL-13 after segmental allergen challenge and their effects on alpha-smooth muscle actin and collagen III synthesis by primary human lung fibroblasts. Clin Exp Allergy 2004; 34:437-44). IL-4 and especially IL-13 act on epithelial cells and induce transcription of specific genes (Zhu Z, et al. Pulmonary expression of interleukin-13 causes inflammation, mucus hypersecretion, subepithelial fibrosis, physiologic abnormalities, and eotaxin production. J Clin Invest 1999; 103:779-88; Striz I, et al. IL-4 induces ICAM-1 expression in human bronchial epithelial cells and potentiates TNFalpha. Am J Physiol 1999; 277:L58-64). Microarray analysis of airway epithelial cells from asthmatic patients demonstrated an increase in IL-4/IL-13-responsive genes (Woodruff P G, et al. T-helper type 2-driven inflammation defines major subphenotypes of asthma. Am J Respir Crit Care Med 2009; 180:388-95). However, a significant number of asthmatic patients did not show an increase in IL-4/IL-13-responsive genes. On the basis of these findings, Woodruff et al. have identified two subgroups of asthma: TH2^{high} and TH2^{low}.

Most differentiated T helper cells manifest plasticity and acquire additional functional features (Mocci S, and Coffman R L. Induction of a Th2 population from a polarized Leishmania-specific Th1 population by in vitro culture with IL-4. J Immunol 1995; 154:3779-87; Yang X O, et al. Molecular antagonism and plasticity of regulatory and inflammatory T cell programs. Immunity 2008; 29:44-56; Koenen H J, et al. Human CD25highFoxp3pos regulatory T cells differentiate into IL-17-producing cells. Blood 2008; 112:2340-52; Lee Y K, Turner, et al. Late developmental plasticity in the T helper 17 lineage. Immunity 2009; 30:92-107; Wei G, et al. Global mapping of H3K4me3 and H3K27me3 reveals specificity and plasticity in lineage fate determination of differentiating CD41 T cells. Immunity 2009; 30:155-67; Zhou L, et al. Plasticity of CD41 T cell lineage differentiation. Immunity 2009; 30:646-55. Th2 cells can acquire the ability to produce Th17 cytokines without losing their ability to produce Th2 cytokines (Cosmi L, et al. Identification of a novel subset of human circulating memory CD4(1) T cells that produce both IL-17A and IL-4. J Allergy Clin Immunol 2010; 125:222-30, e1-4). The dual-positive cells emerge from Th2 cells in the presence of Th17− inducing cytokines: IL-1b, IL-6, and IL-21 (Wang Y H, et al. A novel subset of CD4(1) T(H)2 memory/effector cells that produce inflammatory IL-17 cytokine and promote the exacerbation of chronic allergic asthma. J Exp Med 2010; 207:2479-91). These dual-positive blood cells also express CCR6. The frequency of the Th2/Th17 population is increased in peripheral blood from asthmatic patients (Wang Y H, et al.). Using a multicolor flow cytometric approach, the inventors detected Th2, Th17, and dual-positive Th2/Th17 cells in BAL fluid from asthmatic patients. The differentiation of Th2 and Th17 is regulated by GATA3 and RORgt, respectively. Immunofluorescence studies of BAL cells showed co-expression of IL-4 and IL-17, as well as nuclear colocalization of GATA3 and RORgt, supporting the Th2/Th17 phenotype. IL-4 and IL-6 play a crucial role in induction of Th2 and Th17, respectively. They do so by inducing phosphorylation of STAT6 and STAT3. The inventors have been able to demonstrate the presence of dual-positive pSTAT3/pSTAT6 cells, further confirming the dual Th2/Th17 phenotype.

One area of interest is the mechanism of differentiation of dual-positive Th2/Th17 cells. Both Th2 and Th17 cells could give rise to Th2/Th17 cells. However, the frequency of single-positive Th17 cells was either less than that of Th2 cells or undetectable in the study subjects. This contrasted with much higher frequency of Th2 cells in BAL fluid from most asthmatic patients. Furthermore, IL-17 production in dual-positive cells was usually associated with IL-4^{high} CD4 T-cell counts. These findings favor Th2 cells as the precursors for Th2/Th17 cells. The generation and sustenance of Th2 cells require the AP1 transcription factor JunB (Hartenstein B, et al. Th2 cell-specific cytokine expression and allergen-induced airway inflammation depend on JunB. EMBO J 2002; 21:6321-9). Some recent publications have demonstrated that JunB forms a trimolecular complex with basic leucine zipper ATF-like (Batf) and interferon regulatory factor (IRF) (Coffman R L, et al. Reversal of polarized T helper 1 and T helper 2 cell populations in murine leishmaniasis. Ciba Found Symp 1995; 195:20-33; Glasmacher E, et al. A genomic regulatory element that directs assembly and function of immunespecific AP-1-IRF complexes. Science 2012; 338:975-80; Li P, et al. BATF-JUN is critical for IRF4-mediated transcription in T cells. Nature 2012; 490:543-6). This complex binds to the so-called AP1-IRF composite element (AICE) in the IL-17 gene promoter and plays an important role in IL-17 gene induction (Ciofani M, et al. A validated regulatory network for Th17 cell specification. Cell 2012; 151:289-303). The inventors have previously shown that JunB is a MEK-inducible protein (Liang Q, et al. IL-2 and IL-4 stimulate MEK1 expression and contribute to T cell resistance against suppression by TGF-beta and IL-10 in asthma. J Immunol 2010; 185:5704-13). Thus IL-4^{high} Th2 cells with a high JunB level could, under Batf-inducing conditions (eg, stimulation with IL-1), begin to form complexes with Batf and IRF4 and lead to IL-17 production. MEK also induces the PEA-3 (polyoma enhancer activator 3) family transcription factor Etv4 (Fontanet P, et al. Pea3 transcription factor family members Etv4 and Etv5 mediate retrograde signaling and axonal growth of DRG sensory neurons in response to NGF. J Neurosci 2013; 33: 15940-51; Li X, et al. MEK is a key regulator of gliogenesis in the developing brain. Neuron 2012; 75:1035-50). The latter is required for Th17 induction (Pham D, et al. The transcription factor Etv5 controls TH17 cell development and allergic airway inflammation. J Allergy Clin Immunol 2014; 134:204-14). Thus MEK-driven JunB and Etv4 may promote IL-17 production in IL-4high Th2 cells.

A previous study has demonstrated that Th2/Th17 cells induce a more severe form of experimental asthma in an adoptive transfer model in mice when compared with Th2 and Th17 cells (Wang et al.). The inventors have observed more severe airway obstruction and airway hyperreactivity in the Th2/Th17^{predominant} subgroup. An explanation for the increased severity of asthma could be the presence of Th2/Th17 cells. However, the mean fluorescence intensity (MFI) of IL-4 in Th2/Th17 cells from the Th2/Th17^{predominant} subgroup is higher than that in Th2 cells from the Th2^{predominant} subgroup. These data are support a higher level of IL-4 production by the Th2/Th17 cells in the Th2/Th17^{predomiant} subgroup. Thus the severity of asthma in this subgroup could be due to increased IL-4 production. On the other hand, IL-17 production by Th2/Th17 cells is likely to change the quality of airway inflammation and function. IL-17 is known to directly affect airway epithelial cells, fibroblasts, and smooth muscle cells (Barczyk A, et al.; Bullens D M, et al.; Molet S, et al.; Al-Ramli W, et al.; and Agache I, et al). In agreement, the inventors have observed a significant negative correlation between BAL IL-17 levels and FEV1. All asthmatic patients were taking high-dose inhaled steroids at the time of the study. Despite this treatment, they had sustained airway obstruction, suggesting relative steroid resistance. Th17 cells have previously been shown to be resistant to glucocorticoids.¹⁴ The inventors' results show that the dual-positive Th2/Th17 cells are also resistant to dexamethasone. This resistance may contribute to the refractory nature of their asthma. A number of molecular mechanisms have been implicated in glucocorticoid resistance. Reduced glucocorticoid receptor (GR) phosphorylation by p38 mitogen-activated protein kinase (α and γ) leads to reduced nuclear translocation and confers resistance (Irusen E, et al., p38 Mitogen-activated protein kinase-induced glucocorticoid receptor phosphorylation reduces its activity: role in steroid-insensitive asthma. J Allergy Clin Immunol 2002; 109:649-57). However, Th17 cells manifest normal nuclear translocation of GR receptor (McKinley L, et al. TH17 cells mediate steroid-resistant airway inflammation and airway hyperresponsiveness in mice. J Immunol 2008; 181:4089-97). The anti-inflammatory action of the GR is facilitated by histone deacetylase 2 (HDAC2), a chromatin-remodeling enzyme. Patients with severe asthma have reduced HDAC2 levels, which could contribute to glucocorticoid resistance (Ito K, et al. Expression and activity of histone deacetylases in human asthmatic airways. Am J Respir Crit Care Med 2002; 166:392-6). The 6 isoform of phosphatidylinositol-3 kinase phosphorylates HDAC2 and contributes to its degradation, which results in steroid resistance (To Y, et al. Targeting phosphoinositide-3-kinase-delta with theophylline reverses corticosteroid insensitivity in chronic obstructive pulmonary disease. Am J Respir Crit Care Med 2010; 182:897-904). The transcription factor c-Fos (a component of AP1) directly antagonizes GR and confers resistance (Adcock I M, et al. Abnormal glucocorticoid receptor-activator protein 1 interaction in steroid-resistant asthma. J Exp Med 1995; 182:1951-8). c-Fos is downstream of the MEK-extracellular signal-regulated kinase 1/2 signaling pathway. The inventors have found that severe asthma is associated with increased expression of MEK1 in CD4 T cells (Liang Q, et al. IL-2 and IL-4 stimulate MEK1 expression and contribute to T cell resistance against suppression by TGF-beta and IL-10 in asthma. J Immunol 2010; 185:5704-13). Increased expression of MEK1 confers resistance against a broad spectrum of endogenous homeostatic regulators (i.e., TGF-β, IL-10, and glucocorticoids) through induction of c-Fos and JunB. Inhibition of MEK1 reverses this resistance. The inventors demonstrate that Th2/Th17 cells express higher levels of MEK1, and these MEK1^{high} CD4 T cells are resistant to dexamethasone. The inventor's BAL CD4 T-cell analysis allows them to identify 3 distinct subgroups of asthma: Th2^{predominant}, Th2/Th17^{predominant}, and Th2/Th17^{low}. Woodruff et al. (2009) identified only Th2^{high} and Th2^{low} subgroups of asthma based on epithelial microarray data. The inventors of the present invention have identified a subgroup of Th2^{predominant} patients in whom BAL Th2 cells co-express IL-17. This finding has mechanistic and clinical implications. It is well known that asthma exacerbation is frequently triggered by respiratory tract infection (Jackson D J, et al. Asthma exacerbations: origin, effect, and prevention. J Allergy Clin Immunol 2011; 128:1165-74). A subgroup of asthmatic patients has chronic lung infections with Mycoplasma and Chlamydia species (Johnston S L, and Martin R J. Chlamydophila pneumoniae and Mycoplasma pneumoniae: a role in asthma pathogenesis? Am J Respir Crit Care Med 2005; 172:1078-89). Many environmental factors, such as air pollution, smoke, and chemicals, nonspecifically aggravate asthma (To T, et al. The air quality health index and asthma morbidity: a population-based study. Environ Health Perspect 2013; 121:46-52; Perez L, et al. Chronic burden of near-roadway traffic pollution in 10 European cities (APHEKOM network). Eur Respir J 2013; 42:594-605). Infections and these environmental factors are known to elicit inflammatory cytokines, such as IL-1b and IL-6 (Tabarani C M, et al. Novel inflammatory markers, clinical risk factors and virus type associated with severe respiratory syncytial virus infection. Pediatr Infect Dis J 2013; 32:e437-42; Thompson A M, et al. Baseline repeated measures from controlled human exposure studies: associations between ambient air pollution exposure and the systemic inflammatory biomarkers IL-6 and fibrinogen. Environ Health Perspect 2010; 118:120-4). Both IL-1b and IL-6 induce differentiation of Th2/Th17 cells from Th2 cells. Thus a typical patient with allergic asthma with a Th2^{predominant} endotype could trend toward a Th2/Th17^{predominant} endotype over time if he or she has recurrent respiratory tract infections, were exposed to the aforementioned environmental toxicants, or both. The invasive nature of bronchoscopy and the potential for adverse events together are a major deterrence for patient participation. Induced sputum, which is less invasive, is an alternative approach to obtaining airway cells. However, the number of cells obtained through induced sputum is much less than that through BAL. Isolation of cells from the sputum requires harsh mucolytic treatment, which can affect cellular function and viability. Flow cytometric analysis of sputum cells has shown greater variability.

Finally, the subjects in the study took routine medications at the time of BAL. Medications can affect T-cell numbers and their cytokine expression. Unfortunately, there is no alternative. The institutional review board does not allow discontinuation of medications, especially in patients with severe asthma, whose disease can deteriorate without medication.

As demonstrated in the examples presented below, the inventors have found an increased frequency of dual-positive Th2/Th17 cells in BAL fluid from asthmatic patients. The increased expression of Th2/Th17 cells and one of its cytokines, IL-17, are associated with heightened airway hyperreactivity and airway obstruction, two objective features of asthma severity. Severe asthma manifests relative steroid resistance. The inventors provide a mechanistic explanation for this resistance. Th2/Th17 cells express high levels of MEK1, which is associated with steroid resistance. The identification of a TH2/TH17^{predominant} endotype in addition to the previously recognized TH2^{predominant} and Th2^{low} endotypes of asthma has pathogenetic and therapeutic implications.

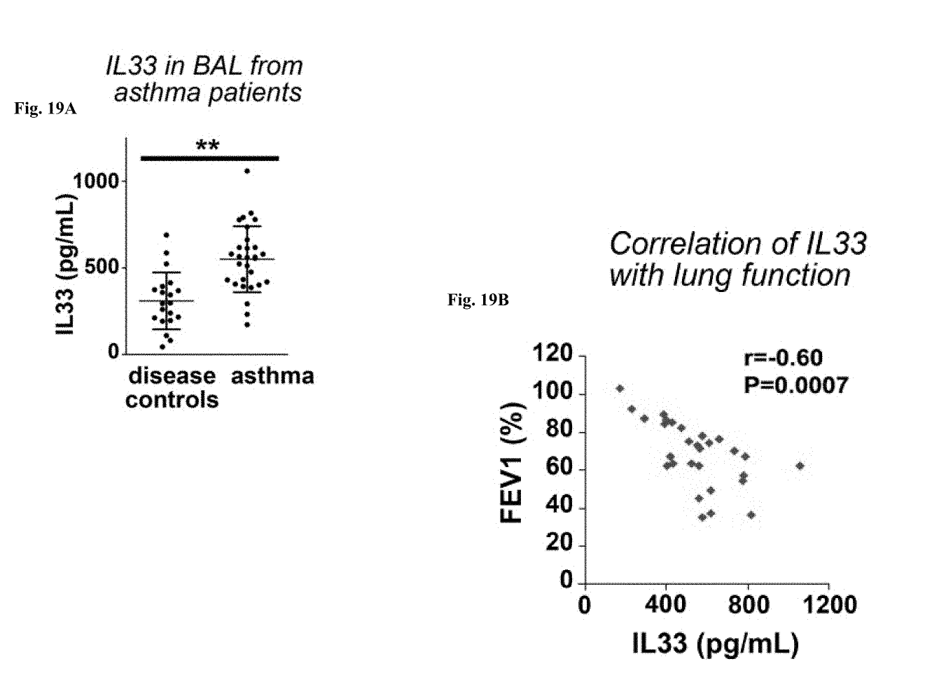

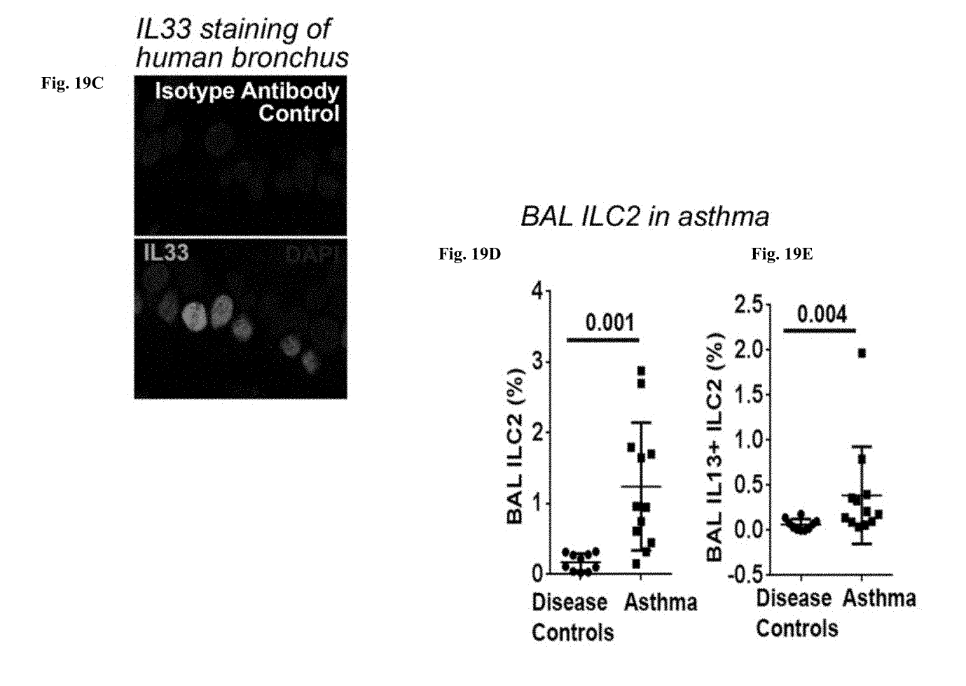

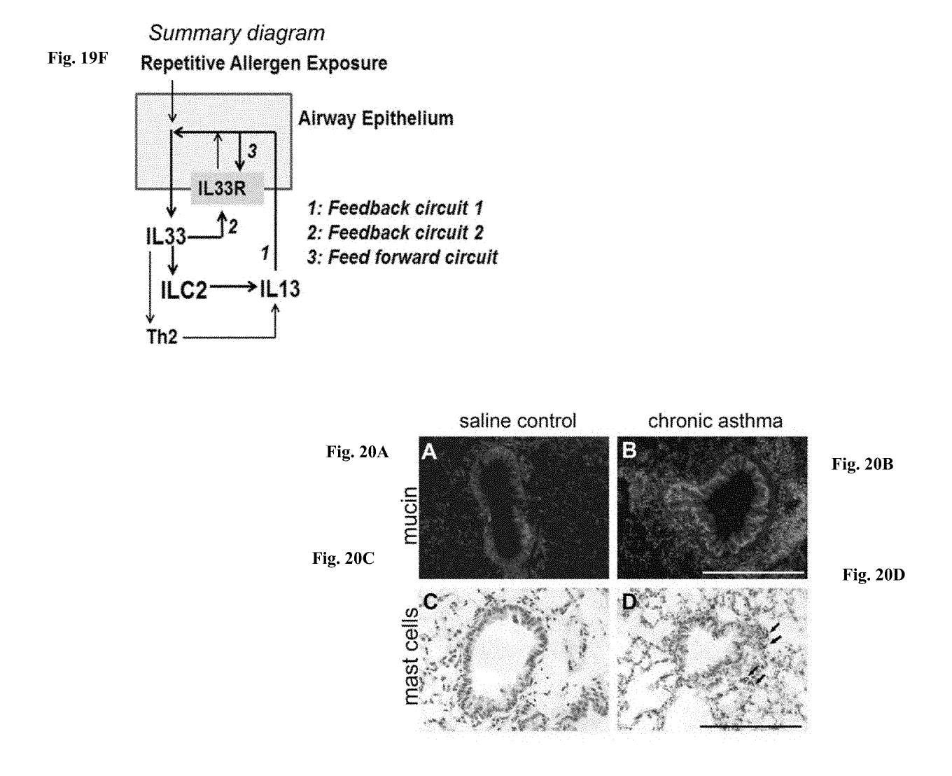

Persistence of asthma is generally considered to be mediated by allergen-specific memory T cells (Corry D B, et al. Requirements for allergen-induced airway hyperreactivity in T and B cell-deficient mice. Mol Med 1998; 4:344-55). The depletion of allergen-specific memory T cells in the model of the inventors did not eliminate airway hyperreactivity and remodeling. However, their absence reduced airway hyperreactivity and especially inflammation. On the other hand, the depletion of ILC2s led to resolution of all features of asthma. Although many previous studies demonstrated the ability of ILC2s to induce airway eosinophilic inflammation independent of T cells, this is the first demonstration of ILC2s inducing sustained airway hyperreactivity. The inventors substantiated the role of ILC2s through gain-in-function (adoptive transfer) and loss-of-function (lethal irradiation followed by Rag2−/−: γc−/− marrow transplantation and anti-IL-33 treatment) approaches. Epithelial cells and ILC2s established three distinct positive feedback and feed-forward mechanisms to sustain asthma, as summarized in FIG. 19F. The outcomes of these interconnected positive feedback and feed-forward circuits were persistent IL-33 production and development of chronic asthma. The importance of the ILC2-driven feedback and feed-forward circuits was illustrated by the loss of IL-33 production in irradiated mice that received Rag2−/−: γc−/−, but not Rag1−/−, marrow. The interdependence of IL-33 and IL-13 was recently reported in a mouse model of virus-induced chronic obstructive pulmonary disease (Byers D E, et al. Long-term IL-33-producing epithelial progenitor cells in chronic obstructive lung disease. J Clin Invest 2013; 123:3967-82).

An important feature of these positive feedback and feedforward circuits is that they are interconnected. There is strong mathematic and experimental evidence that interconnected positive feedback circuits induce ultrasensitivity and bistability (Chang D E, et al. Building biological memory by linking positive feedback loops. Proc Natl Acad Sci USA 2010; 107:175-80; Shin S Y, et al. Functional roles of multiple feedback loops in extracellular signal-regulated kinase and Wnt signaling pathways that regulate epithelial-mesenchymal transition. Cancer Res 2010; 70:6715-24). Ultrasensitivity is manifested when a linear input generates a sigmoidal output. Ultrasensitivity induces system bistability. A system is considered bistable when it is “on” (active) in the absence of any input (Xiong W, and Ferrell J E Jr. A positive-feedback-based bistable ‘memory module’ that governs a cell fate decision. Nature 2003; 426:460-5; Markevich N I, et al. Signaling switches and bistability arising from multisite phosphorylation in protein kinase cascades. J Cell Biol 2004; 164:353-9; Srividhya J, et al., Open cascades as simple solutions to providing ultrasensitivity and adaptation in cellular signaling. Phys Biol 2011; 8:046005). The effect of interconnected feed-forward circuits is illustrated by the differential effect of IL-13 versus IL-4 or IL-17 on IL-33 autoinduction. Although all three cytokines directly induced IL-33, only IL-13 produced synergy through its feed-forward effect on IL-33R expression. Previously, the inventors demonstrated that repetitive stimulation of epithelial cells with IL-13 led to extracellular signal-regulated kinase (ERK) 1/2 bistability through the establishment of a signaling feedback circuit (LiuW, et al. Establishment of extracellular signal-regulated kinase 1/2 bistability and sustained activation through Sprouty 2 and its relevance for epithelial function. Mol Cell Biol 2010; 30:1783-99). ERK1/2 bistability primed epithelial cells for heightened cytokine production. ERK1/2 signaling is likely relevant because its phosphorylation is increased in airway epithelial cells from asthmatic patients (Liu W, et al. Cell-specific activation profile of extracellular signal-regulated kinase 1/2, Jun N-terminal kinase, and p38 mitogen-activated protein kinases in asthmatic airways. J Allergy Clin Immunol 2008; 121:893-902.e2). Thus IL-13 could establish multiple feedback and feed-forward circuits for sustained IL-33 production.

The inventors have demonstrated the biological relevance of their findings in human asthma. In agreement with previous reports, (Prefontaine D, et al. Increased expression of IL-33 in severe asthma: evidence of expression by airway smooth muscle cells. J Immunol 2009; 183:5094-103; Prefontaine D, et al. Increased IL-33 expression by epithelial cells in bronchial asthma. J Allergy Clin Immunol 2010; 125:752-4), the inventors showed increased IL-33 levels in BAL fluid from asthmatic patients. The IL-33 level negatively correlated with the airway flow volume. For the first time, the inventors demonstrate a significant increase in ILC2 numbers in BAL fluid from asthmatic patients.

Previously, increased ILC2 numbers were reported in nasal polyps from patients with chronic rhinosinusitis (Mj€osberg J M, et al. Human IL-25- and IL-33-responsive type 2 innate lymphoid cells are defined by expression of CRTH2 and CD161. Nat Immunol 2011; 12:1055-62; Shaw J L, et al. IL-33-responsive innate lymphoid cells are an important source of IL-13 in chronic rhinosinusitis with nasal polyps. Am J Respir Crit Care Med 2013; 188:432-9) and in peripheral blood from asthmatic patients (Bartemes K R, et al. Enhanced innate type 2 immune response in peripheral blood from patients with asthma. J Allergy Clin Immunol 2014; 134:671-8.e4). The frequency in polyps ranged from 0.1% to 3.6% of CD45+ cells and that in blood ranged from 0.01% to 0.03% of mononuclear cells. The frequency of ILC2s in BAL fluid from asthmatic patients was similar to that in polyps but was higher than that in peripheral blood.

The novel findings of the inventors as provided and discussed herein and presented in the examples below have implications for chronic illnesses in general. These results indicate that recurrent episodes of any acute illness can establish feedback and feed-forward circuits. Once established, these feedback circuits sustain the disease process during intervals between acute episodes, which facilitates the transition of an acute illness to a chronic one.

The following examples are provided for illustrative purposes, and are not intended to limit the scope of the invention as claimed herein. Any variations which occur to the skilled artisan are intended to fall within the scope of the present invention. All references cited in the present application are incorporated by reference herein to the extent that there is no inconsistency with the present disclosure.

### EXAMPLES

### Example 1

In this example the expression of Th2, Th17, and dual-positive Th2/Th17 cells in BAL fluid from patients with treatment-refractory asthma and their response to glucocorticoids was analyzed. In addition, the clinical relevance of Th2/Th17 cells in asthmatic patients is shown.

### Methods

### Processing of BAL Fluid Cells and Flow Cytometry

Patients were allowed to continue their routine medication. Bronchoscopy and BAL were performed, as described previously (Good J T Jr, et al. Refractory asthma: importance of bronchoscopy to identify phenotypes and direct therapy. Chest 2012; 141:599-606). BAL fluid was processed immediately. Cells were isolated by means of centrifugation. Supernatant fluid was placed into aliquots in small samples and frozen. Cells were either cultured or fixed immediately in 4% paraformaldehyde and processed for flow cytometry. For cultures, cells were washed and divided into 2 treatment groups: medium or dexamethasone (10⁷ mol/L). The cells were cultured in RPMI 1640 with 10% FBS overnight. The next day, the cells were washed and fixed in paraformaldehyde. Monensin (2 μmol/L) was added 6 hours before fixing.

### Flow Cytometry

Cells were stained with the following antibodies. Allophycocyaninlabeled mouse anti-human CD4 (clone RPA-T4), phycoerythrin (PE)-Cy7-labeled mouse anti-human IL-4 (clone MP4 25D2), allophycocyanin/Cy7-labeled mouse anti-human IL-17 (clone BL168), mouse anti-CD3ε (clone OKT3), mouse anti-CD68 (clone Y1/82A), mouse anti-CD163 (clone GH1/61), anti-chemoattractant receptor-homologous molecule expressed on TH2 cells (CRTH2; clone BM16), mouse anti-CCR6 (clone G034E3), and rat anti-IL-5 (clone TRFK5) were from BioLegend (San Diego, Calif.). PE-labeled anti-mitogen-activated protein-extracellular signal-regulated kinase kinase 1 (MEK1; clone 25/MEK1), peridinin-chlorophyll-protein complex-Cy5.5-labeled mouse anti-signal transducer and activator of transcription (STAT) 3 (pY705) (clone 4/P-STAT3), and Alexa Fluor 488-labeled mouse anti-STAT6 (pY641; clone 18/P-STAT6) were from BD Biosciences (San Jose, Calif.). A rabbit anti-mitogen-activated protein kinase phosphatase 1 (MKP1) antibody was from Santa Cruz Biotechnology (Dallas, Tex.). This was detected with an Alexa Fluor 488-labeled anti-rabbit secondary antibody. The isotype controls were rat IgG1 for the anti-IL-4 antibody, mouse IgG1 for anti-CD4 and anti-IL-17 antibodies, and mouse IgG2a for anti-phosphorylated signal transducer and activator of transcription (pSTAT) 3 and anti-pSTAT6 antibodies. The Fc receptors were blocked by incubating cells first with 10% goat serum and then conducting immunostaining with specific antibodies in 5% goat serum. Flow cytometry was performed with a CyAn ADP Analyzer 9 color flow cytometer (Beckman Coulter, Brea, Calif.), as described previously (Liang Q, et al. IL-2 and IL-4 stimulate MEK1 expression and contribute to T cell resistance against suppression by TGF-beta and IL-10 in asthma. J Immunol 2010; 185:5704-13). Flow cytometric data were analyzed with FlowJo software (Tree Star, Ashland, Ore). Only small and nongranular cells were gated by means of forward and side scatter, respectively, and the large and highly granular BAL cells were excluded from analysis because they tend to bind many antibodies nonspecifically, as reported previously (Thomas S Y, et al. Invariant natural killer T cells in bronchial asthma. N Engl J Med 2006; 354:2613-6). The threshold line for identification of positively stained cells was set conservatively based on the control isotype antibody staining pattern. The emphasis was on exclusion of nonspecifically stained cells. Less than 1% (usually <0.5%) of the cells stained positively by using this control antibody-based thresholding strategy. Specific cell populations were first identified (CD4, CD3, CD163, and CD68) and then the cells were analyzed for the presence of intracellular cytokines. Isotype control antibodies were run in all experiments, and the aforementioned gating and thresholding strategy was applied to all.

### BAL Samples

Double immunofluorescence staining BAL cells were fixed, and cytospin preparations were immunostained with a combination of mouse monoclonal anti-GATA3 (clone TWAJ; eBioscience, San Diego, Calif.) and a rabbit polyclonal anti-retinoic acid receptor-related orphan receptor gt (RORgt; clone H-190, Santa Cruz Biotechnology) antibody or mouse anti-IL-4 (clone 8D4-8; BD PharMingen, San Jose, Calif.) and rabbit anti-IL-17 (Santa Cruz Biotechnology) antibodies, as described previously (Guo L, et al. Nuclear translocation of MEK1 triggers a complex T cell response through the corepressor silencing mediator of retinoid and thyroid hormone receptor. J Immunol 2013; 190:159-67). Fluorescein isothiocyanate- and PE-labeled secondary antibodies were directed against anti-GATA3 and anti-RORgt antibodies, respectively. The cytospin preparations were counterstained with 49-6-diamidino-2-phenylindole dihydrochloride (DAPI). Z-series images were captured with a Zeiss confocal microscope (Zeiss, Oberkochen, Germany).

### ELISA

IL-17 (IL-17A) was assayed in undiluted BAL fluid by using an ELISA kit from R&D Systems (Minneapolis, Minn.), according to the supplier's instructions.

### Statistical Analyses

Comparisons between study groups were done with the Mann-Whitney U test. Comparisons among multiple study groups were performed by using the Kruskal-Wallis test. The Pearson correlation coefficient was used to calculate the correlation.

### Results

These results demonstrate the detection of single TH2 and TH17 cells and dual TH2/TH17 cells in BAL fluid from asthmatic patients.

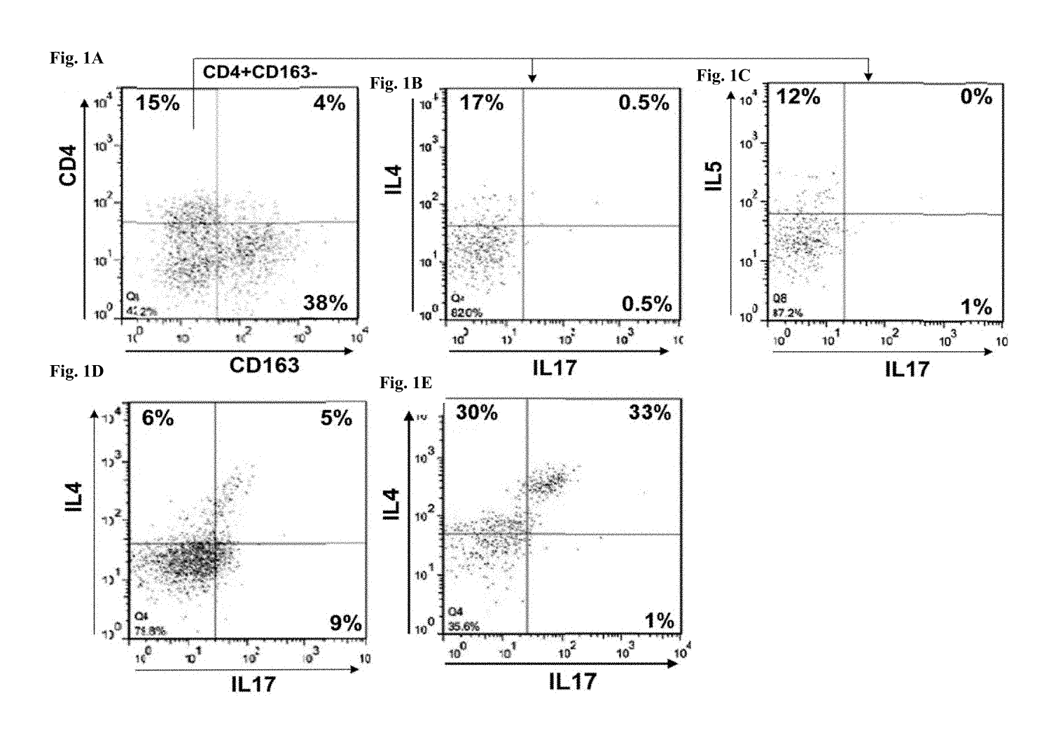

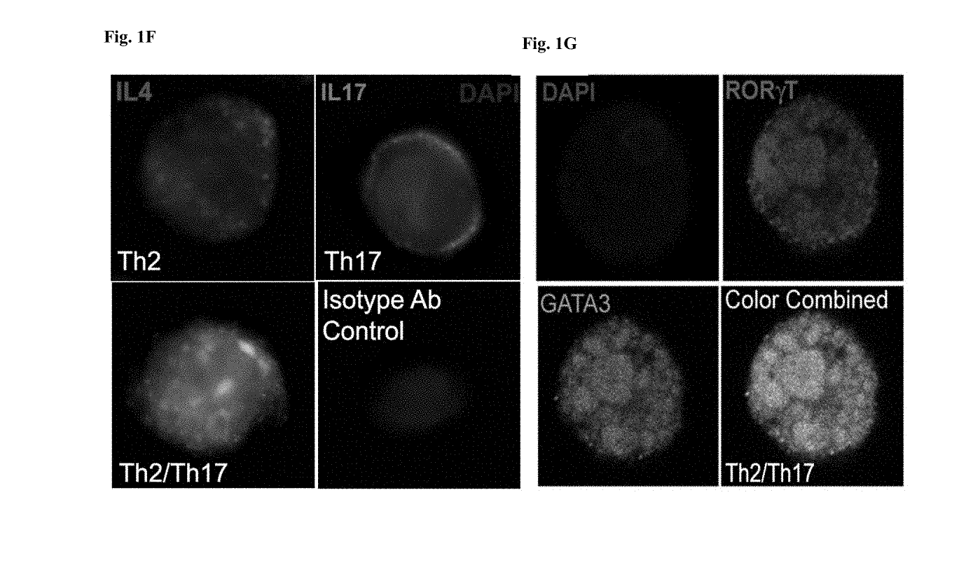

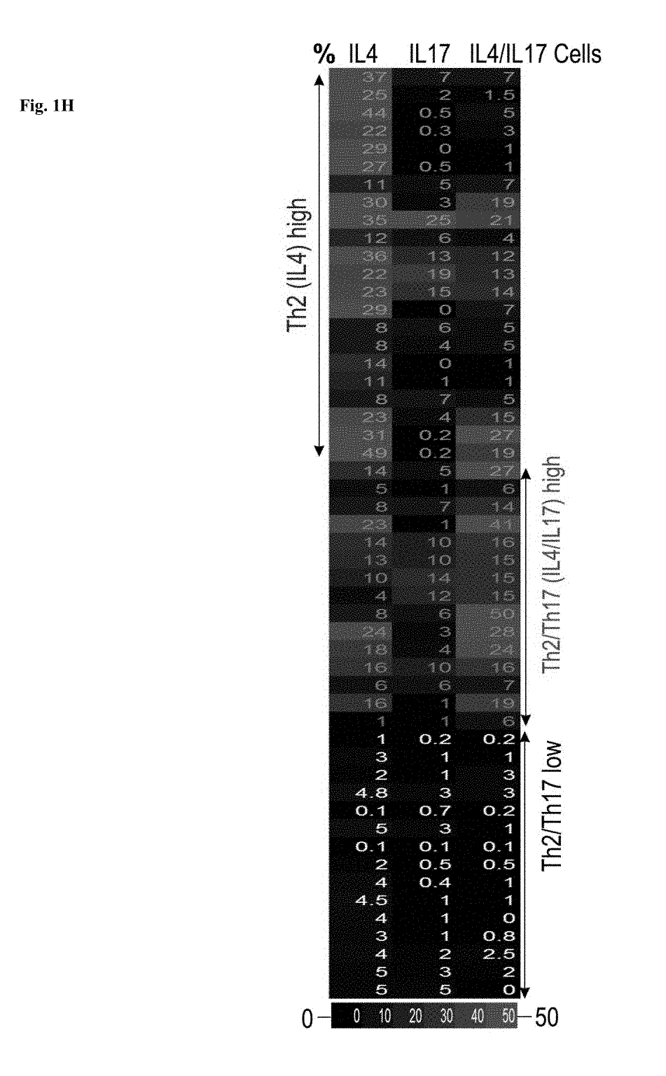

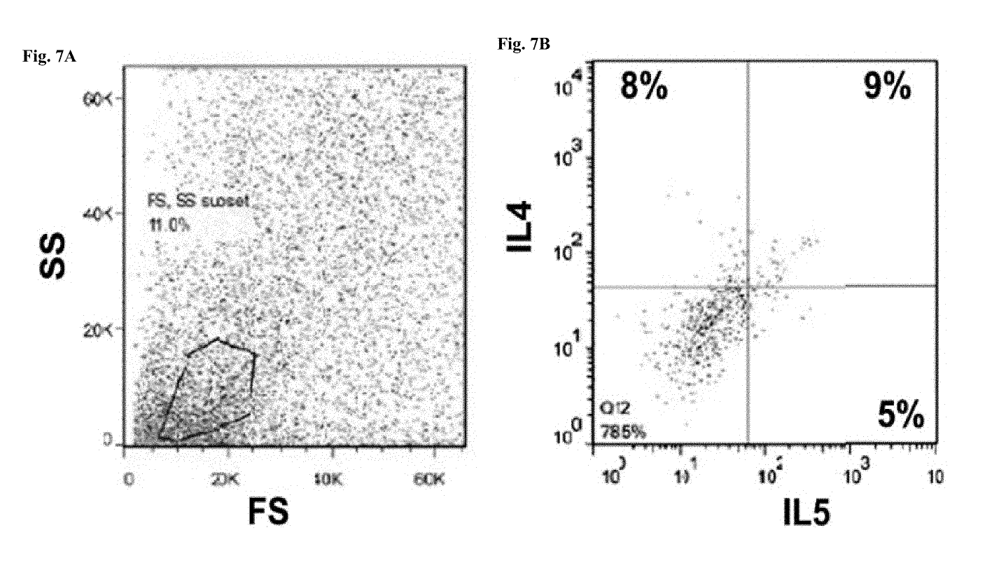

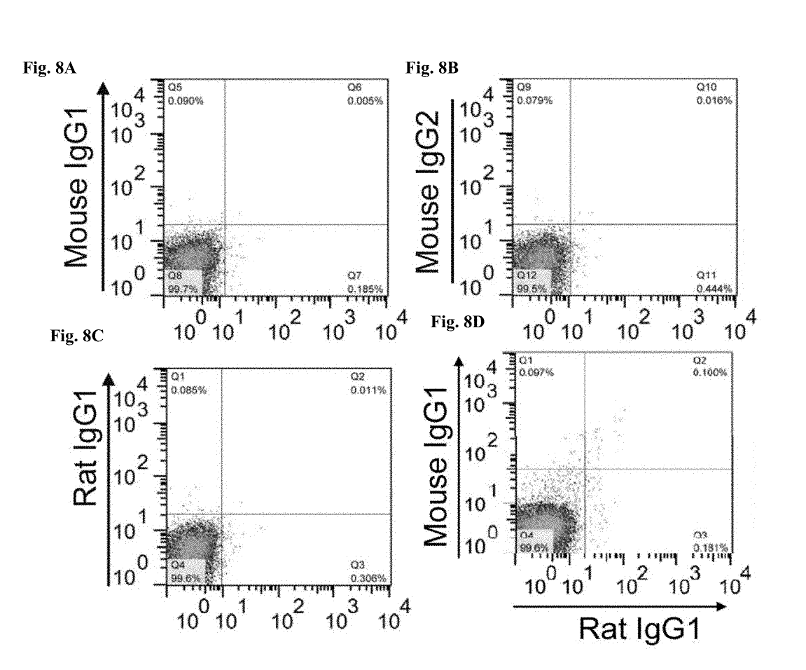

BAL cells from 52 asthmatic patients and 25 disease control subjects were studied. Most of the patients were referred for diagnosis and management of refractory asthma. Others were referred for routine asthma care. The term refractory as used herein indicates uncontrolled asthma, which could be moderate or severe, as determined based on the Expert Panel Report 3 criteria. The clinical characteristics of the study subjects are shown in Table 1. Bronchoscopy and BAL were performed, as described previously (Good J T Jr, et al. Refractory asthma: importance of bronchoscopy to identify phenotypes and direct therapy. Chest 2012; 141:599-606). BAL fluid was processed immediately for flow cytometry or for culture overnight, as described below. After immunostaining, cells were first gated for small nongranular cells by means of forward and side scatter (see FIG. 7A). Next, cells positive for CD4 and negative for the macrophage marker CD163 were identified (FIG. 1A). These CD4+CD63− cells were analyzed for intracellular IL-17 and IL-4 or IL-5. FIGS. 1B and 1C, shows detection of largely IL-41 and IL-51 CD4 T cells in BAL fluid from an asthmatic patient. Very few CD4 T cells stained for IL-17. Coexpression of IL-4 and IL-5 was analyzed in these BAL cells. Partial overlap between IL-41 and IL-51 cells was observed (see FIG. 1B), suggesting both synchronous and nonsynchronous production of these 2 TH2 cytokines. FIG. 1D, represents a BAL flow cytogram with a TH17^{predominant} pattern, which comes from a patient with chronic pulmonary aspiration. FIG. 1E, represents a TH2/TH17^{predominant} staining pattern obtained from an asthmatic patient. Flow cytograms of BAL cells with isotype control antibodies are shown in FIG. 8A through 8D.

TABLE 1
Characteristics of the study subjects

| Parameters | Asthmatic patients | | Disease controls | |
|---|---|---|---|---|
| N | 52 | | 25 | |
| Diagnoses | 52 patients with asthma; | | 16 patients with chronic cough | |
| | co-morbidities: 41 patients | | and concurrent allergic rhinitis | |
| | with allergic rhinitis, 29 with | | & GERD, 3 patients with | |
| | chronic sinusitis, 38 with | | bronchiectasis, 5 with chronic | |
| | GERD, 3 with bronchiectasis | | aspiration and 1 with COPD | |
| | and 5 with aspiration | | | |
| Male/female | 23/29 | | 12/13 | |
| Age | 52.88 ± 3 | (52) | 52.2 ± 3 | (53) |
| FEV1 (%) | 71 ± 2 | (71.2^{α})* | 93.5 ± 4 | (88.5) |
| Reversibility (%) | 18.0 ± 2.2 | (13.5)* | 2.1 ± 0.6 | (1) |
| PC20 (mg/ml) for | 2.95 ± 0.3 | (2.2)* | 24.5 ± 0.5 | (25) |
| methacholine | | | | |
| Eosinophils/μL blood | 336 ± 50 | (300)* | 100 ± 17 | (100) |
| Total IgE (KIU/L) | 215 ± 46 | (99) | 165 ± 78 | (36) |
| BMI | 28.7 ± 1 | (30) | 25.3 ± 1 | (25) |

^{α}number in the parenthesis indicates median;
*P < 0.05, Mann-Whitney U test

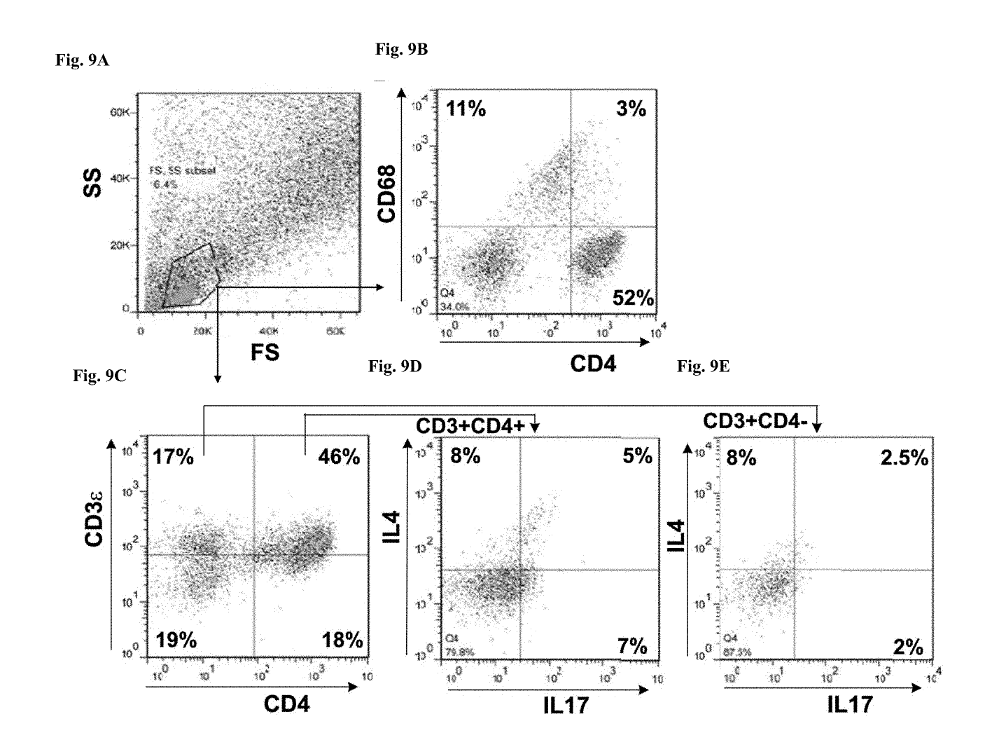

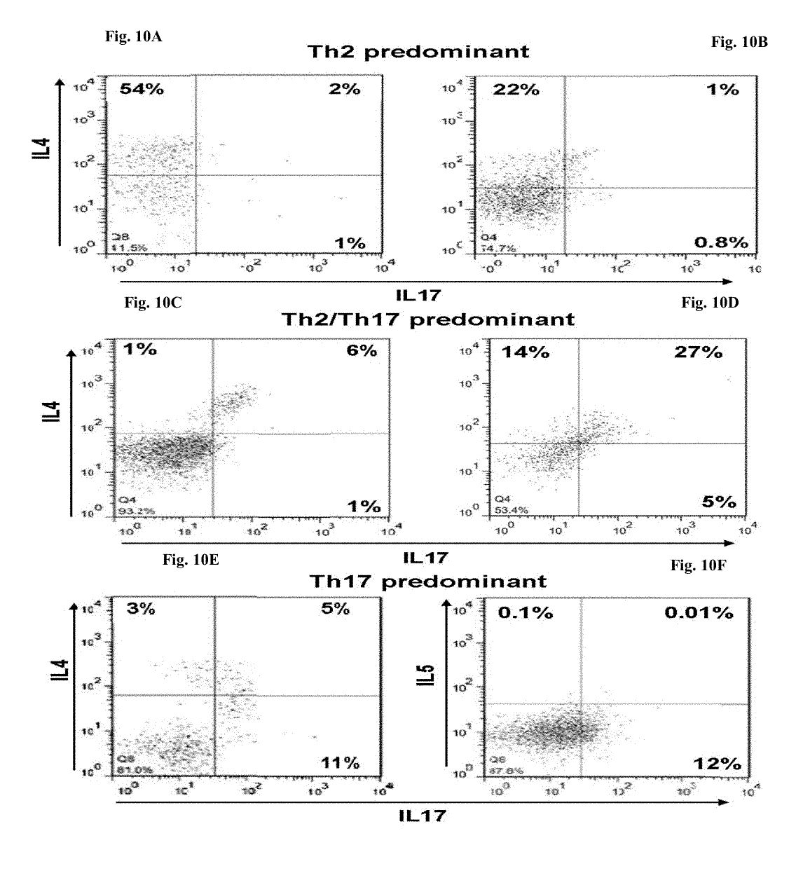

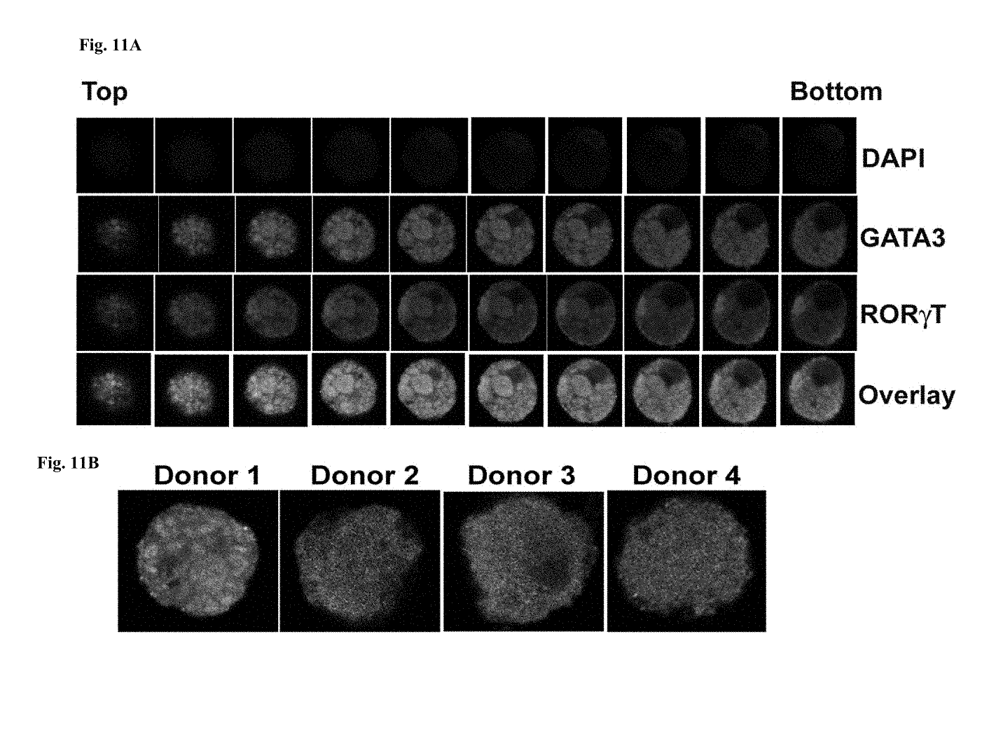

In another approach BAL cells for CD4 and CD68 were analyzed (a different macrophage marker) or CD4 and CD3ε(see FIG. 9A-9C) The vast majority of CD4 cells were CD68-CD3ε+, supporting their T-cell origin. There was a smaller population of cells that were CD4-CD3ε+, which are likely CD8 and γδ T cells. Both CD4+CD3+ and CD4-CD3+ T-cell populations contained variable numbers of IL-41 and IL-171 cells and dual-positive IL-4/IL-17 cells (see FIGS. 9D and 9E). In general, 2 staining patterns were observed. In one pattern the cytokine-positive cells were clearly separated from the nonstained cytokine-negative cells (see FIGS. 10A and 10B, for IL-4; FIGS. 1D and 1E, and 10C, for IL-4/IL-17; and FIG. 10E, for IL-17). In another pattern the cytokine-positive cells showed a small but measurable shift in staining intensity, which made them positive for cytokine expression, but they did not clearly separate from the cytokine-negative cells (see FIGS. 1B, 1C, and 1E, for IL-4; FIG. 10D, for IL-4/IL-17; and FIGS. 1D, and 10F, for IL-17). The dual-positive IL-4/IL-17 cells were usually better separated from the cytokine-negative cells and showed a higher level of mean fluorescence intensity (MFI). FIG. 1F, presents a heat map profile of IL-4+ and IL-17+ cells and dual-positive IL-4/IL-17 CD4 T cells in BAL fluid from all 52 asthmatic patients, as studied by using flow cytometry. The heat map demonstrates 3 different BAL CD4 T-cell profiles in asthmatic patients: (1) Th2 (IL-4)^{predominant}, (2) Th2/Th17 (IL-4/IL-17)^{predominant}, and (3) Th2/Th17^{low}. To confirm the flowcytometric result of coexpression of IL-4 and IL-17, immunocytochemical staining of BAL cells was performed (FIG. 1G). The frequency of cytokine-positive cells in 3 different BAL samples was 22%±7%. Cells positive for IL-4 or IL-17 was detected, as well as cells coexpressing IL-4 and IL-17. These cells were relatively small in size and round in shape, with a large nucleus and small perinuclear cytoplasm. IL-4 showed a large vesicular staining pattern. In contrast, IL-17 presented a diffuse and small vesicular staining pattern. The staining of both single- and double-positive cells in the same BAL specimen further validated the staining specificity of the used antibodies.

### Coexpression of GATA3 and RORgt in BALlymphocytes

Differentiation of Th2 and Th17 cells is controlled by the transcriptional regulators GATA3 and RORγt, respectively. Co-expression of GATA3 and RORgt was examined using immunofluorescence staining and confocal microscopy in BAL lymphocytes from 4 asthmatic patients. FIG. 1H, shows GATA3 and RORγt staining patterns of a representative BAL lymphocyte from an asthmatic patient. This image represents the midsection from a Z-series. The entire Z-series images from this patient and the representative image from all 4 donors are shown in FIG. 11. Staining with the DNA-binding dye DAPI shows the nuclear staining pattern. The less dense and transcriptionally active euchromatin stained light blue, whereas the tightly packed and transcriptionally inactive heterochromatin stained dark blue. Both GATA3 and RORγt stained primarily the nucleus. The staining was most prominent with euchromatin and negligible with heterochromatin.

Expression of Dual-Positive IL-4/IL-17 Cells is Associated with Dual-Positive pSTAT6/pSTAT3- and CCR6/CRTH2-Expressing Cells

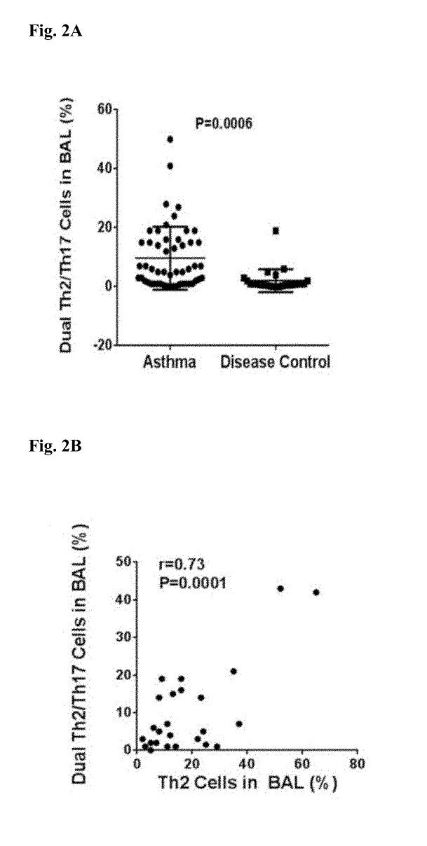

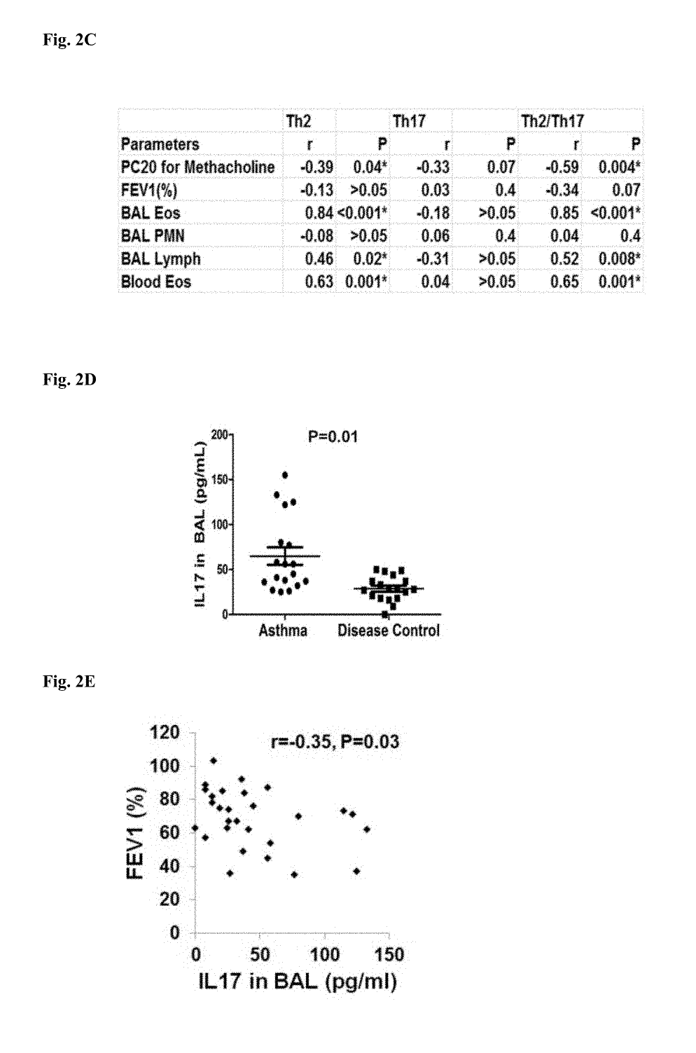

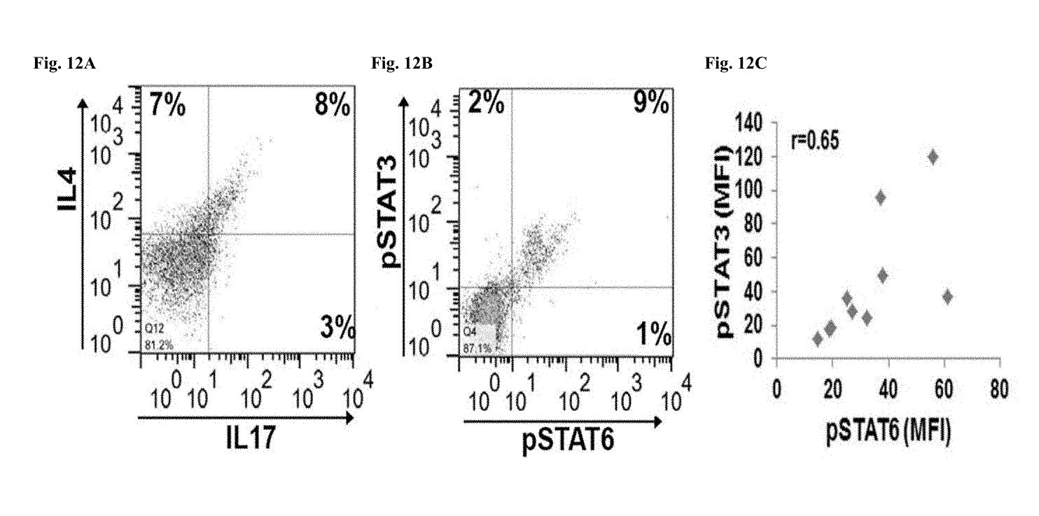

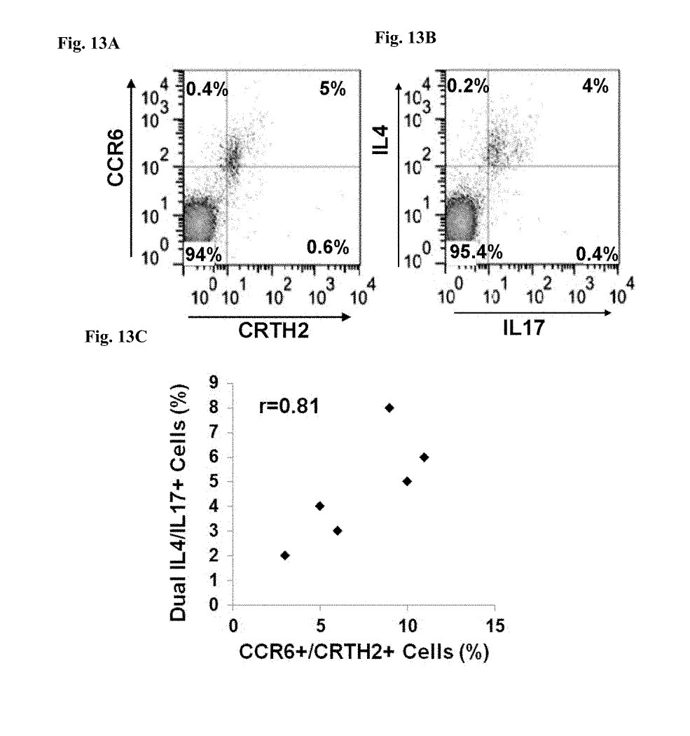

The phosphorylation and activation of STAT6 by IL-4 and STAT3 by IL-6 and IL-21 are early events during differentiation of TH2 and TH17 cells (Kaplan M H, and Grusby M J. Regulation of T helper cell differentiation by STAT molecules. J Leukoc Biol 1998; 64:2-5; Yang X O, et al. STAT3 regulates cytokine-mediated generation of inflammatory helper T cells. J Biol Chem 2007; 282:9358-63). The expression of activating phosphorylation of STAT3 and STAT6 in BAL CD4 T cells was examined. Significant coexpression of pSTAT3 and pSTAT6 was observed, which followed the pattern of IL-4 and IL-17 coexpression (see FIGS. 12A and 12B). There was a strong correlation between pSTAT3 and pSTAT6 levels (see FIG. 12C). These results suggest that the phosphorylation of STAT3 and STAT6 is an active and ongoing process in the BAL milieu from some asthmatic patients. Dual-positive IL-4/IL-17 cells in the peripheral blood express the Th2 marker CRTH2 and the Th17 marker CCR6 (Wang Y H, et al. A novel subset of CD4(1) T(H)2 memory/effector cells that produce inflammatory IL-17 cytokine and promote the exacerbation of chronic allergic asthma. J Exp Med 2010; 207:2479-91). In agreement, CCR6+CRTH2+ cells in BAL fluid was detected (see FIG. 13A). The frequency of CCR6+CRTH2+ cells correlated with IL-4/IL-17+ cells (see FIGS. 13B and 13C). Increased expression of dual-positive Th2/Th17 cells correlates with airway hyperreactivity, blood and BAL eosinophil counts, and BAL lymphocyte counts. The inventors compared the expression of BAL dual-positive Th2/Th17 cells between 52 asthmatic patients and 25 disease control subjects. The disease control subjects included 16 patients with chronic cough and a concurrent diagnosis of allergic rhinitis and gastroesophageal reflux disease refractory to treatment. Additional control subjects included 3 patients with bronchiectasis, 5 patients with chronic aspiration, and 1 patient with chronic obstructive pulmonary disease. The number of dual-positive cells was significantly (P=0.0006) increased in asthmatic patients compared with that seen in disease control subjects (FIG. 2A). The median frequency of dual-positive (IL-4 and IL-17) cells was 5% and 1% in asthmatic patients and disease control subjects, respectively. The median frequency of single-positive IL-4 CD4 T cells was 11% and 1% in asthmatic patients and disease control subjects, respectively. Th2/Th17 cells could differentiate from either single-positive Th2 cells or single-positive Th17 cells. A previous study showed that Th2 cells differentiated intodual-positive Th2/Th17 cells in vitro under the influence of Th17-inducing cytokines (Wang et al. 2010). A strong correlation between Th2 and Th2/Th17 cells was observed (FIG. 2B), supporting this notion. Both Th2 and Th2/Th17 cell numbers correlated negatively with bronchial hyperreactivity (PC20 for methacholine), and positively with BAL and blood eosinophil counts, and BAL lymphocyte counts (FIG. 2C). In each case the correlation was stronger for TH2/TH17 cells. There was no correlation with FEV1 or BAL neutrophil counts.

IL-17 Levels are Increased in BAL Fluid from Asthmatic Patients and Negatively Correlate with FEV1

Since expression of IL-17 by Th2/Th17 cells was detected, levels of secreted IL-17 in BAL fluid using ELISA was measured. The IL-17 level was significantly increased in asthmatic patients compared with that seen in disease control subjects (FIG. 2D). The IL-17 level negatively correlated with FEV1 (as a percentage) in asthmatic patients (FIG. 2E). This correlation is not unexpected because IL-17 enhances airway smooth muscle contraction (Kudo M, et al. IL-17A produced by alphabeta T cells drives airway hyper-responsiveness in mice and enhances mouse and human airway smooth muscle contraction. Nat Med 2012; 18: 547-54). Note that the Th2/Th17 number showed a trend for correlation with FEV1 but did not reach statistical significance (P=0.07). It is likely that the amount of secreted IL-17 is variable among Th2/Th17 cells, which could help explain this minor discrepancy.

Dexamethasone does not Inhibit BAL-Derived Dual-Positive Th2/Th17 Cells

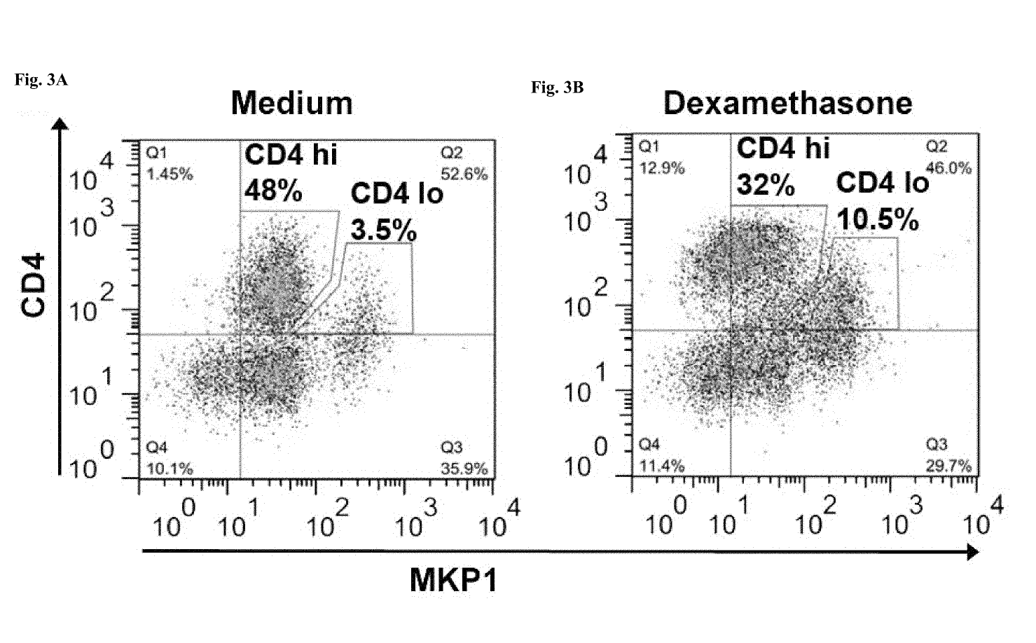

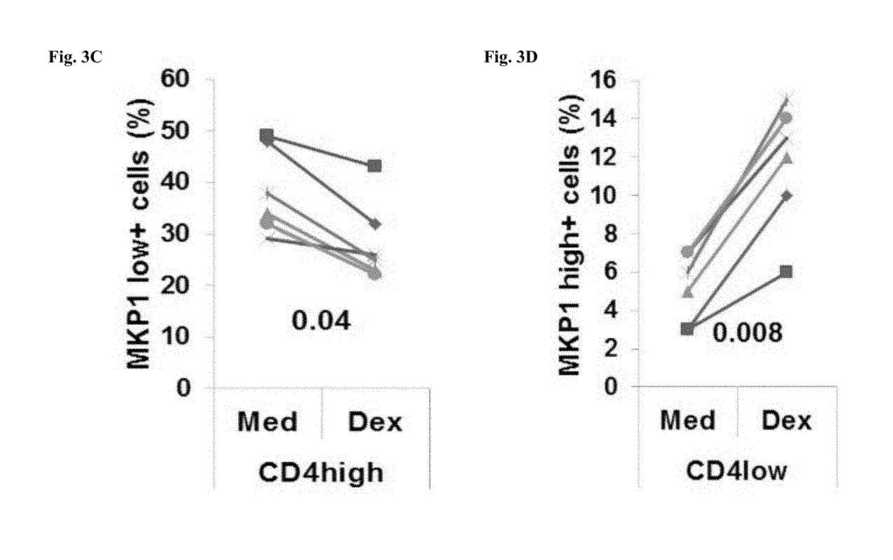

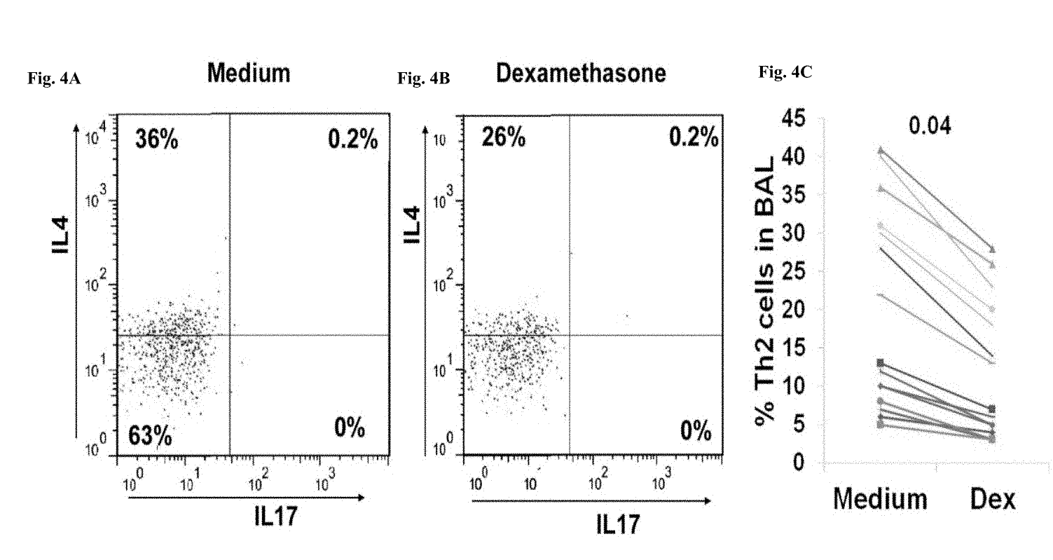

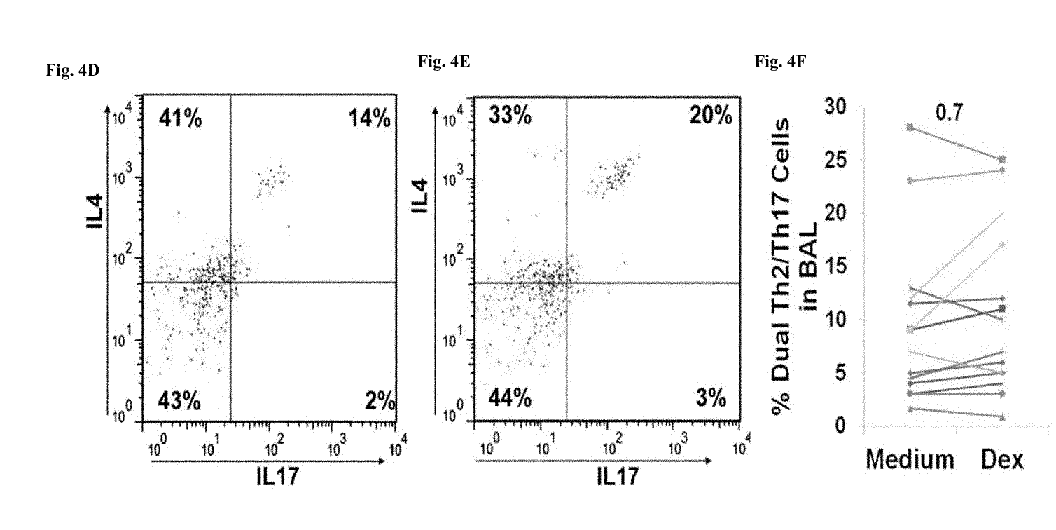

Most of the patients were referred for refractory asthma and relative steroid resistance. For this reason, sensitivity of Th2/Th17 to dexamethasone was examined. One of the dexamethasone-responsive genes is MKP1 (Kassel O, et al. Glucocorticoids inhibit MAP kinase via increased expression and decreased degradation of MKP-1. EMBO J2001; 20:7108-16). Differential expression of MKP1 in T cells after treatment with dexamethasone was observed. Dexamethasone increased the number of cells that expressed higher levels of MKP1 (FIGS. 3A and 3B). This population distinguished itself by expressing an intermediate level of CD4. On the other hand, the number of T cells that expressed high levels of CD4 and low levels of MKP1 decreased after dexamethasone. FIGS. 3C and 3D, shows a similar differential pattern of dexamethasone response in 6 study subjects. These studies suggest that different populations of CD4 T cells respond differently to dexamethasone. Next, the effect of dexamethasone on Th2, Th17, and dual-positive Th2/Th17 cells was studied. Dexamethasone downregulated IL-4+Th2 cells (FIG. 4) but did not show any inhibitory effect on dual-positive Th2/Th17 cells (FIGS. 4C, 4D, and 4F). Thus Th2/Th17 cells are relatively resistant to the apoptotic effect of dexamethasone. Dexamethasone actually increased the number of Th2/Th17 cells in some patients.

### BAL Dual-Positive Th2/Th17 Cells Express Higher Levels of MEK1

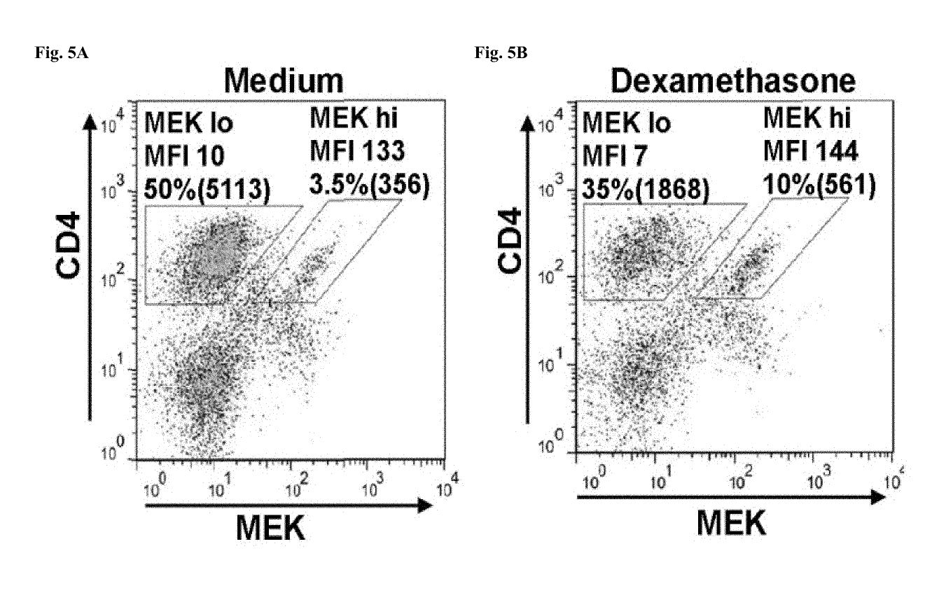

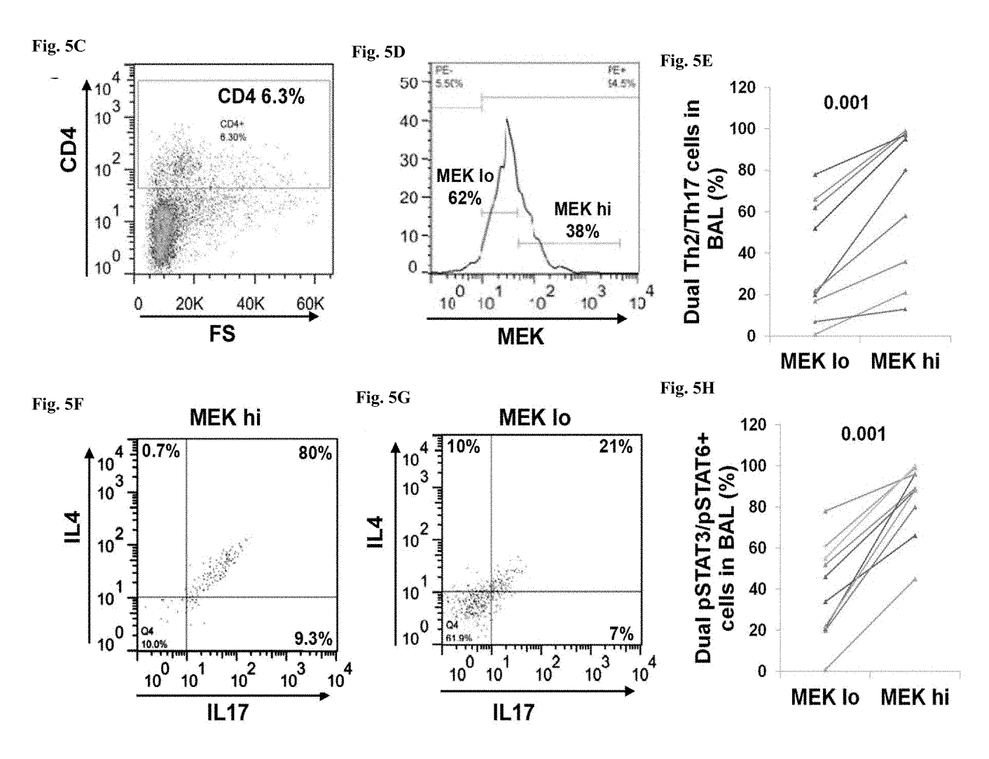

One of the signaling pathways that antagonize the inhibitory action of glucocorticoids is the MEK-extracellular signal-regulated kinase 1/2 pathway (Liang Q, et al. IL-2 and IL-4 stimulate MEK1 expression and contribute to T cell resistance against suppression by TGF-beta and IL-10 in asthma. J Immunol 2010; 185:5704-13; Tsitoura D C, and Rothman P B. Enhancement of MEK/ERK signaling promotes glucocorticoid resistance in CD41 T cells. J Clin Invest 2004; 113:619-27; Adcock I M, et al. Abnormal glucocorticoid receptor-activator protein 1 interaction in steroid-resistant asthma. J Exp Med 1995; 182:1951-8). This pathway induces the activating protein 1 (AP1) transcription factors, which antagonize glucocorticoids. Conversely, glucocorticoids antagonize AP1 by inducing the glucocorticoid-induced leucine zipper (Mittelstadt P R, Ashwell J D. Inhibition of AP-1 by the glucocorticoid-inducible protein GILZ. J Biol Chem 2001; 276:29603-10). The inventors have previously reported that CD4 T cells from patients with moderate-to-severe asthma have increased expression of MEK1 (Ling et al. 2010). Inhibition of MEK1 reverses T-cell resistance against dexamethasone. BAL CD4 T cells for MEK1 (referred to as MEK) expression and the sensitivity of MEK1 cells to dexamethasone was analyzed. A small CD4 population in BAL fluid that expressed a high level (MFI 133) of MEK was observed (MEKhi; FIG. 5A). There was a larger population of CD4 T cells that expressed a low level (MFI 10) of MEK (MEK^{low}). The MEK^{high} CD4 T-cell population was completely resistant to dexamethasone-induced cell death (FIGS. 5A and 5B). This contrasted with the MEK^{low} population, whose cell count decreased by 2.7-fold after dexamethasone treatment. Next, the inventors determined whether MEK expression was differentially regulated in dual-positive Th2/Th17 cells. To this objective, the MEK^{high} and MEK^{low} cell populations were analyzed (FIGS. 5C and 5D) for enrichment of TH2/TH17 cells. MEK^{high} CD4 T cells were shown to be disproportionately enriched for dualpositive Th2/Th17 cells (FIG. 5E-5G). In contrast, the MEK^{low} Tcell population had significantly reduced numbers of Th2/Th17 cells. The mean frequency of TH2/TH17 cells was 66%+/−12% (median, 80%) in the MEK^{high} population compared with 36%+/−9% (median, 22%) in the MEK^{low} population. The MEK^{high} population was also enriched for dual-positive pSTAT3/pSTAT6 cells (FIG. 5H).

### The Th2/Th17predominant Subgroup has the Most Severe Airway Hyperreactivity and Obstruction

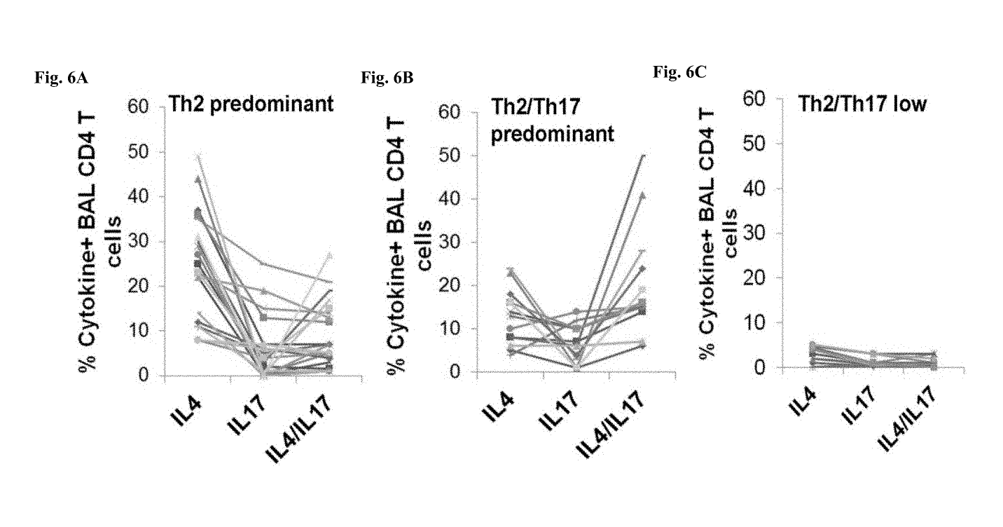

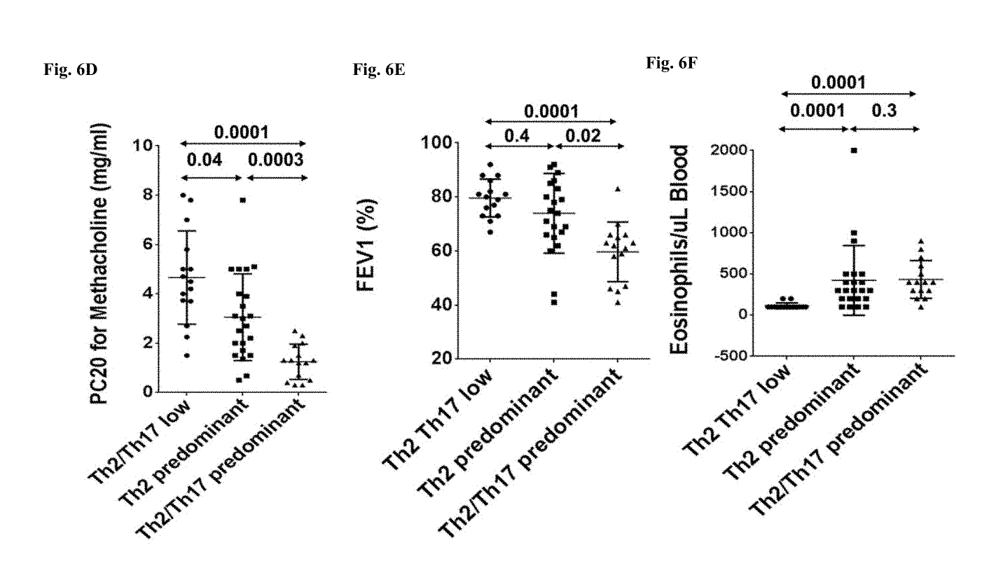

Based on the frequency of Th2 and Th2/Th17 cells in BAL fluid, the population can be divided into 3 separate subgroups (FIG. 6A-6C): (1) Th2^{predominant}, (2) Th2/Th17^{predominant}, and (3) Th2/Th17^{low}. The Th2^{predominant} subgroup (22 patients) had the highest frequency of single-positive IL-4 cells compared with the Th2/Th7^{predominant} and Th2/Th1710 w groups (Table 2). The Th2/Th17^{predominant} subgroup (15 patients) had the highest number of dual-positive IL-4/IL-17 cells compared with the Th2^{predominant} and Th2/Th17^{low} groups. The Th2/Th17^{loW} subgroup (15 patients) had 5% or less of each of these cell populations in BAL fluid. Interestingly, although the TH2/TH17^{predominant} group had fewer IL-41 cells than the TH2^{predominant} group (12.6%+/−2% vs 22.7%+/−2%), the MFI of IL-4 in IL-4/IL-171 cells in this group was higher (304+/−65 vs 180+/−85), suggesting that these cells produce higher quantities of IL-4 on a per-cell basis.

TABLE 2
Comparison of clinical features of Th2/Th17^{low}, Th2^{predominant} and
Th2/Th17^{Predominant} subgroups of asthmatic patients

| Parameters | Th2/Th17^{low} | | Th2^{predominant} | | Th2/Th17^{Predominant} | |
|---|---|---|---|---|---|---|
| N | 15 | | 22 | | 15 | |
| Co-morbidities: | | | | | | |
| Allergic rhinitis | 11 | | 19 | | 8 | |
| Chronic sinusitis | 4 | | 7 | | 7 | |
| GERD | 9 | | 9 | | 6 | |
| Bronchiectasis | 0 | | 1 | | 0 | |
| Aspiration | 0 | | 1 | | 1 | |
| Smoking | 0 | | 1 | | 0 | |
| % Cytokine+ cells in the | IL4: 3.0 ± 0.5 (3.5) | | IL4: 22.7 ± 2 (23)Ω | | IL4: 12.6 ± 2 (13) | |
| gated BAL cell population | IL17: 1.2 ± 0.3 (1) | | IL17: 5.9 ± 1.6 (4) | | IL17: 6.5 ± 1.5 (6) | |
| Mean ± SEM (Median) | IL4/IL17: 1.1 ± 0.3 | | IL4/IL17: 7.3 ± 1.5 (5) | | IL4/IL17: 20.4 ± 4(16)* | |
| | (1) | | | | | |
| MFI of Cytokine+ cells in | ND | | IL4: 125 ± 43 (68) | | IL4: 103 ± 11 (92) | |
| the gated BAL cell | | | IL17: 34 ± 7 (27) | | IL17: 34 ± 2 (34) | |
| population | | | IL4 in IL4/IL17+ | | IL4 in IL4/IL17+ cells: | |
| | | | cells: 180 ± 85 (83) | | 304 ± 65(304)# | |
| Positive skin test | 8 | | 19 | | 12 | |
| Total IgE (KIU/L) | 111 ± 26 | (100) | 123 ± 26 | (98) | 230 ± 53 | (167) |
| BMI | 26.5 ± 2 | (27) | 29.0 ± 1.8 | (30) | 25.9 ± 1.2 | (26) |
| FEV1 (%) | 79.6 ± 2 | (80) | 73.9 ± 3 | (74) | 59.6 ± 2.7 | (62)*#α |
| PC20 (mg/ml) for | 4.6 ± 0.5 | (4.5) | 2.9 ± 0.4 | (2.85)∞ | 1.24 ± 0.2 | (1.3)*#β |
| methacholine | | | | | | |
| Eosinophils/μL blood | 113 ± 9 | (100) | 422 ± 85 | (200)∞ | 433 ± 57 | (400)*μ |
| Asthma medications | | | | | | |

| | | | |
|---|---|---|---|
| systemic steroids | 1 | 2 | 4 |
| omalizumab | 0 | 1 | 3 |
| ICS/LABA | 15 | 22 | 15 |
| LTI | 2 | 5 | 1 |
| SABA | 15 | 22 | 15 |
| Tiotropium | 1 | 1 | 0 |

*P < 0.05 compared to Th2^{predominant} and Th2/Th17^{low};
#P < 0.05 compared to Th2^{predominant};
ΩP < 0.05 compared to Th2/Th17^{predominant} and Th2/Th17^{low};
∞P < 0.05 compared to Th2/Th17^{low}, Mann-Whitney U test;
αP = 0.0004;
βP = 0.00007;
μP = 0.002 all Kruskal Wallis test for multiple comparisons.
Abbreviations:
ND: not done;
PC20: provocation concentration causing a 20% drop in FEV1;
ICS: inhaled corticosteroid;
LABA: long-acting beta-adrenergic agonist;
LTI: leukotriene receptor inhibitor; and
SABA: short-acting beta-adrenergic agonist

Next, clinical and biochemical features of the identified subgroups were examined. Airway hyperreactivity was most severe in the Th2/Th17^{predominant} subgroup (FIG. 6D). The PC20 for methacholine were 1.24±0.2, 2.9±0.4 and 4.6±0.5 mg/ml in the Th2/Th17^{predominant}, Th2^{predominant} and Th2/Th17^{low} subgroups, respectively. The differences were statistically significant. Airway obstruction was most severe in the Th2/Th17^{predominant} subgroup compared with the other subgroups (FIG. 6E). FEV1 values were 59.6%+/−2.7%, 73.9%+/−3%, and 79.6%+/−2% in the Th2/Th17^{predominant}, Th2^{predominant}, and Th2/Th17^{low} subgroups, respectively. The differences were statistically significant except for TH2^{predominant} versus Th2/Th17^{low}. Blood eosinophilia was present in both the TH2^{predominant} and Th2/Th17^{predominant} subgroups but was absent in the Th2/Th17^{low} subgroup (FIG. 6F). There were no differences in total IgE levels among the subgroups. All study subjects were taking high-dose inhaled corticosteroid/long-acting bronchodilator therapy. The number of patients receiving chronic systemic steroid therapy and omalizumab was highest in the Th2/Th17^{predominant} group.

### Example 2

This example studies the mechanisms of the persistence of asthma in the absence of the inciting allergens. To accomplish this, a mouse model in which asthma persisted for 3 weeks after cessation of repetitive dust mite, ragweed, and Aspergillus species allergen exposure was used (Bullens D M, et al. IL-17 mRNA in sputum of asthmatic patients: linking T cell driven inflammation and granulocytic influx? Respir Res 2006; 7:135).

### Methods

### Animals

C57Bl/6 CD45.1, C57Bl/6 CD45.2, BALB/c, Rag1−/−, Rag2−/−: γc−/− and Erk1−/− mice were used in this study.

### Mucosal Sensitization of Mice

Allergens used included extracts of dust mite (Dermatophagoides farinae), ragweed (Ambrosia artemisiifolia), and Aspergillus fumigatus (Greer Laboratories, Lenoir, N.C.). Intranasal allergens (dust mite, 5 μg; ragweed, 15 μg; Aspergillus species, 5 μg [DRA]) or Aspergillus species (5 μg) were delivered in 15-mL quantities in saline. Acute asthma was produced by means of immunization of 8- to 12-weekold BALB/c mice twice 1 week apart with Aspergillus species (5 μg) in adjuvant (1:1 vol/vol). Adjuvant was aluminum and magnesium hydroxide (Pierce, Rockford, Ill.). Asthma was initiated by using 3 consecutive intranasal exposures to Aspergillus species (5 μg in 15 mL of saline), and asthma was evaluated 72 hours after the final exposure.

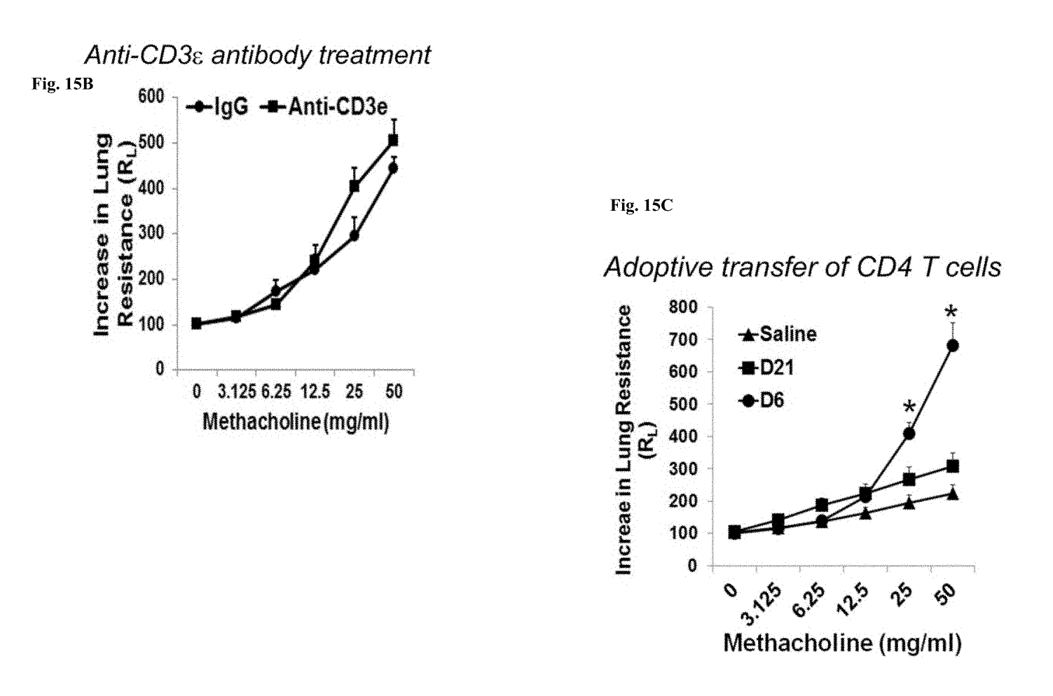

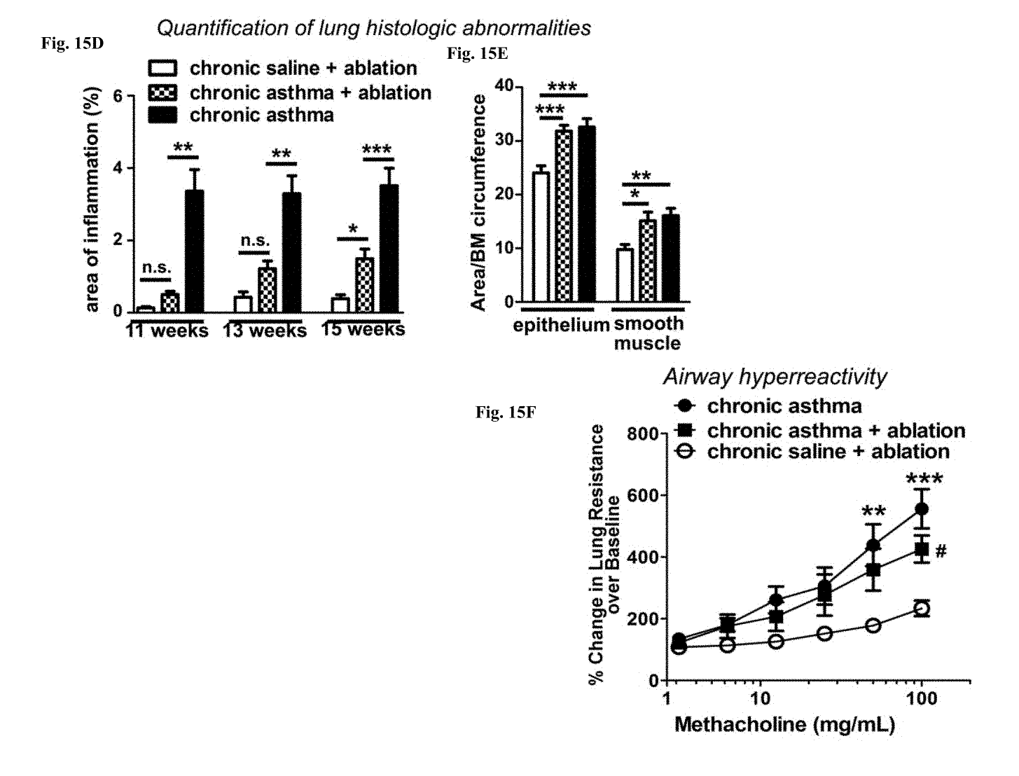

Chronic asthma was produced by intranasal delivery of the DRA mixture twice a week for 6 consecutive weeks in female mice (C57Bl/6 CD45.2 or BALB/c mice, as appropriate) 8 to 12 weeks of age. For characterization of asthma chronicity experiments, mice were analyzed at the indicated time points (1, 2, or 6 months after cessation of allergen exposure). A timeline of manipulations and interventions for the chronic asthma protocol with irradiation is shown in FIG. 15A. For irradiation experiments, mice were rested for 3 weeks after completion of allergen delivery, and 2 doses of 600 rad were delivered 3 hours apart. Flow cytometric analysis indicates that 6 weeks after irradiation, less than 4% of CD45.2+ cells from recipient mice were still present. Bone marrow cells (5×10⁶) from C57Bl/6 CD45.1, BALB/c Rag1−/−, or BALB/c Rag2−/−: γc−/− mice, as indicated in the experimental design, were transferred intravenously, and mice were rested an additional 6 weeks before analyses.

### Antibody, Pharmacologic, and Genetic Interventions

For IL-33 blockade experiments, intraperitoneal anti-IL-33 (15 μg/200 mL injection in saline; R&D Systems, Minneapolis, Minn.) was delivered 3, 5, and 7 days before analysis in week 15 (6 weeks after irradiation). All non-irradiated control mice with asthma were rested an equivalent amount of time before analyses. For CD3 and IL-13 blockade, hamster anti-mouse CD3ε (200 μg per dose, clone 145 2C11; eBioscience, San Diego, Calif.) (Haile S, et al. Mucous-cell metaplasia and inflammatory-cell recruitment are dissociated in allergic mice after antibody- and drug-dependent cell depletion in a murine model of asthma. Am J Respir CellMol Biol. 1999; 20:891-902) and anti-IL-13 (50 μg per dose; Calbiochem, San Diego, Calif.) antibodies and hamster IgG or rat IgG were administered intraperitoneally on 3 consecutive days in week 10, and outcomes were examined 3 days later.

### Adoptive Transfer of CD4 T Cells and ILC2s

Spleen CD4 T cells were negatively selected by using antibody-coated magnetic beads (Miltenyi Biotec, Bergisch Gladbach, Germany) and transferred intravenously (4×10⁶ cells) to naive mice. ILC2s (lin-CD45+CD25+) were sorted on MoFlow XDP (Beckman Coulter, Fullerton, Calif.) and delivered intravenously (2×10⁵ cells) to naive mice 24 hours after sorting. Intranasal DRA (5 μg, 15 μg, and 5 μg, respectively, per 15-mL dose in saline) was performed on 3 consecutive days after intravenous transfer of CD4 T cells. CD4 T cell-recipient mice were studied on days 6 and 21. ILC recipient mice were analyzed on day 21.

### Airway Hyperreactivity Measurement

Measurement methodologies have been explained in depth in Goplen et al. (Goplen N, et al. Combined sensitization of mice to extracts of dust mite, ragweed, and Aspergillus species breaks through tolerance and establishes chronic features of asthma. J Allergy Clin Immunol 2009; 123:925-32.e11). Briefly, mice were anesthetized with ketamine (180 mg/kg), xylazine (9 mg/kg), and acepromazine (4 mg/kg). After loss of footpad pinch reflex, a tracheotomy was performed, and the mouse was attached with an 18-gauge cannula to a small-animal ventilator with a computer-controlled piston (FlexiVent; SCIREQ, Montreal, Quebec, Canada). Mice were ventilated at a frequency of 90 breaths/min with a tidal volume of 20 mL/kg during nebulization and otherwise with a frequency of 150 breaths/min with a tidal volume of 20 mL/kg while breathing against an artificial positive endexpiratory pressure of 2.5 to 3 cm H₂O. Lungs were inflated to total lung capacity twice to standardize volume history. Resistance measurements were then taken to establish baselines for total lung resistance and at each methacholine dose. Group averages were expressed as fold increases in baseline resistance (means+SEMs).

### Histology and Immunofluorescence Staining

Paraffin-embedded lungs were sectioned and stained with hematoxylin and eosin (H&E) for morphometric analysis or mucin staining or toluidine blue for mast cell staining. Sections for immunofluorescence staining were permeabilized with 0.01% saponin in PBS, blocked with 10% goat serum, and stained with a primary antibody against human IL-33 and visualized with Alexa Fluor 488-conjugated secondary antibody, as described in Gorska et al. (Gorska M M, et al. MK2 controls the level of negative feedback in the NF-kappaB pathway and is essential for vascular permeability and airway inflammation. J Exp Med 2007; 204:1637-1652) and counterstained with 49-6-diamidino-2-phenylindole dihydrochloride for nuclear staining.

### Morphometric Measurements

Inflammation was quantified by using Metamorph image acquisition and analysis software on H&E-stained lung sections at 20× magnification, as described in Goplen et al. 2009. Airway epithelial hypertrophy and peribronchial smoothmuscle hypertrophyweremeasured as the area of epithelial or smoothmuscle per circumference of airway basement membrane. Airway inflammation was measured as the area of inflammatory infiltrates as a percentage of the total field. A minimum of 5 airways per mouse and 5 to 9 mice per group were quantified. Images were acquired on a Nikon Eclipse TE2000-U microscope by using 320 dry lenses at room temperature through a Diagnostics Instruments camera model #4.2 with Spot software 5.0 (Diagnostic Instruments, Inc, Sterling Heights, Mich.). H&E sections were mounted with Permount medium (Thermo Fisher Scientific, Inc, Waltham, Mass.). Images were adjusted for brightness and contrast to improve viewing.

### Lung Digestions

Lungs were perfused with saline, and single-cell suspensions of lung cells were acquired by using mechanical mincing of lungs followed by digestion at 37° C. for 45 minutes in RPMI with 10% FBS, 1% penicillin/streptomycin, and collagenaseA (1 mg/mL). Cell suspensions were agitated at room temperature for 10 minutes in RPMI with 100 U/mL DNAse I before filtration through 40-mm filters and red blood cell lysis. Single-cell suspensions were subsequently fixed in 4% paraformaldehyde for flow cytometric analysis.

### Flow Cytometric Analyses of ILC2s and Other Cell-Surface Markers

Mouse ILC2s. For mouse flow cytometric staining, all conjugate antibodies were purchased from BioLegend (San Diego, Calif.), unless otherwise stated. Mouse lung digest cells were fixed with 4% paraformaldehyde and incubated with 10% donkey serum and 1% 2.4G2. ILCs were stained with PerCp/Cy5.5-labeled CD45.2 (clone 104), phycoerythrin/Cy7-labeled CD45.1 (clone A20), or both, as appropriate; Pacific Blue-labeled lineage marker antibodies (CD3, Ly-6G/Ly-6C, CD1 b, CD45R/B220, TER-119/erythroid cells); Alexa Fluor 488-conjugated CD25 (clone PC61); allophycocyanin-labeled IL-5 (TRFK5); and phycoerythrin-labeled IL-13 (clone eBio13A, eBioscience). ILC2s were initially characterized with the addition of the primary antibody anti-IL-33R (ST-2; clone AF1004, R&D Systems), followed by allophycocyanin-labeled secondary antibody against the primary antibody and allophycocyanin-eFluor 780-conjugated IL-7Ra (clone eBioRDR5, eBioscience), allophycocyanin-labeled CD117 (ckit; clone ACK2), phycoerythrin-labeled Ly-6A/E (Sca-1; clone D7), phycoerythrin-Cy7-labeled NK1.1 (clone PK136), and allophycocyaninlabeled FcεRI (clone MAR-1).

Lung IL-331 cells. IL-33 levels and localizations were characterized with the primary antibody against IL-33 (clone Poly5165), followed by fluorescein isothiocyanate-labeled secondary and primary antibodies against pro-surfactant protein C (AB3786; Millipore, Temecula, Calif.), followed by allophycocyanin-labeled secondary or phycoerythrin/Cy7-labeled CD11b (clone M1/70) or the primary antibody against E-cadherin (clone H108; Santa Crux Biotechnology, Dallas, Tex.), followed by a Pacific Blue-labeled secondary antibody or allophycocyanin-labeled FcεRI (clone MAR-1).

Human BAL Fluid ILC2s.

BAL fluid cells were fixed in 4% paraformaldehyde and incubated in 10% goat serum. ILC2s were stained with Pacific Blue-labeled lineage marker antibodies (CD3, CD14, CD16, CD19, CD20, and CD56), fluorescein isothiocyanate-labeled IL-7Ra (clone A019D5), PerCp/Cy5.5-labeled IL-13 (clone JES10-5A2), or phycoerythrin-labeled IL-5 (clone JES1-39D10). IL-33R was identified with the addition of a primary antibody against IL-33R (ST-2; clone HB12; MBL International, Woburn, Mass.), followed by allophycocyanin-labeled secondary antibody against the primary antibody. After washing, cells were analyzed by using flow cytometry with the CyAn ADP cytometer (Beckman Coulter). Data were analyzed with FlowJo version 7.6.5 software (TreeStar, Ashland, Ore).

### T Helper Cell Proliferation and Cytokine Production.

Splenocytes and mediastinal lymph node cells (2×10^{6/}mL) were cultured for 96 hours on anti-CD3 and anti-CD28 (1 μg/mL)-coated 48-well plates or stimulated separately with the following allergens: dust mite (10 μg/mL), ragweed (50 μg/mL), and Aspergillus species (10 μg/mL) (Gorska et al. 2007). In one set of cultures, cells were labeled with 1 μmol/L carboxyfluorescein succinimidyl ester (CFSE; Invitrogen, Carlsbad, Calif.). For measurement of cytokines (IL-2 and IL-4), monensin was added to another culture set 6 hours before the conclusion. T cells were detected by addition of allophycocyanin-labeled anti-CD4 (clone GK1.5, BioLegend) before flow cytometric analysis on the CyAn ADP cytometer (Beckman Coulter) for proliferation and intracellular cytokine expression.

### Western Blotting.

Immediately after flexiVent analysis, lung sections were collected in RIPA buffer (50 mmol/L Tris [pH 7.4], 150 mmol/L NaCl, 1 mmol/L EDTA, 1 mmol/L NaF, 1 mmol/L Na3VO4, and 0.1 mmol/L phenylmethylsulfonyl fluoride) containing protease and phosphatase inhibitors and homogenized. Lysed samples were subjected to 10% SDS-PAGE, transferred to a polyvinylidene difluoride membrane, and then immunoblotted with primary antibodies (IL-33 [R&D Systems], tubulin [H-235], and b-actin [Santa Cruz Biotechnology, Dallas, Tex]). After washing, the membranes were incubated with a horseradish peroxidase-conjugated secondary antibody. After additional washing, the membranes were developed with ECL reagent, as previously described (Gorska et al. 2007). A549 cell culture A549 cells were cultured in RPMI in 10% FBS (HyClone; Thermo Scientific, Waltham, Mass.) and 1% penicillin/streptomycin (Gibco, Carlsbad, Calif.) and maintained at 378C in a humidified 5% CO2 incubator. All cytokines were purchased from PeproTech (Rocky Hill, N.J.). In one experiment cells were treated with medium, IL-1b (2 ng/mL), IL-4 (10 ng/mL), IL-13 (20 ng/mL), IL-17 (10 ng/mL), IL-33 (20 ng/mL), TNF (2 ng/mL), IFN-g (10 ng/mL), and TNF (2 ng/mL) plus IFN-g (10 ng/mL) for 72 hours. Cellular RNA was analyzed for mRNA for IL-33 and IL-33R. In a second experiment cells were pretreated for 24 hours with medium, IL-1b (2 ng/mL), IL-4 (10 ng/mL), IL-13 (20 ng/mL), IL-17 (10 ng/mL), TNF (2 ng/mL), or IFN-g (10 ng/mL) before stimulation for 72 hours with IL-33 (20 ng/mL). Cellular RNAwas analyzed for mRNA for IL-33. In a third experiment cells were treated for 24 hours with DRA (5 μg/mL dust mite, 15 μg/mL ragweed, and 5 μg/mL Aspergillus species). Cellular RNA was analyzed for mRNA for IL-33. In a fourth experiment cells were treated for 24 hours with IL-13 (20 ng/mL) and ATP, and the supernatant was collected and analyzed for IL-33 levels by means of ELISA, according to the manufacturer's instructions (R&D systems).

### Real-Time PCR.

Total RNA was isolated from frozen tissues by using Trizol (Invitrogen) or from cells by using a kit (Purelink Mini RNA kit, Life Technologies), and cDNA was synthesized with an ImProm-II cDNA synthesis kit (Promega, Madison, Wis.), according to the manufacturer's instructions, as described previously (Gorska et al. 2007). Gene-specific PCR products were amplified with SYBR Green (Thermo Scientific) and primers outlined in Table 3 by using an Applied Biosystems 7000 Sequence Detection System (Applied Biosystems, Foster City, Calif.). The levels of target gene expression were normalized to glyceraldehyde-3-phosphate dehydrogenase (GAPDH) expression by using the 2^{-ΔΔ}Ct method.

TABLE 3
Nucleotide Sequence of the primers used for
real-time PCR

| primer | | |
|---|---|---|
| name | SEQ ID NO: | Sequence 5′→3′ |
| mIL4 F | SEQ ID NO: 1 | TTGAGAGAGATCATCGGCATTT |
| mIL4 R | SEQ ID NO: 2 | CTCACTCTCTGTGGTGTTCTTC |
| mIL7 F | SEQ ID NO: 3 | CACACTCACGTCCAGATTTAG |
| mIL7 R | SEQ ID NO: 4 | TCCTAGCCTGCCTTAGATC |
| mIL25 F | SEQ ID NO: 5 | CCAGCAAAGAGCAAGAAC |
| mIL25 R | SEQ ID NO: 6 | TTCAAGTCCCTGTCCAAC |
| mIL33 F | SEQ ID NO: 7 | TCCCAACAGAAGACCAAAG |
| mIL33 R | SEQ ID NO: 8 | GATACTGCCAAGCAAGGAT |
| mTSLP F | SEQ ID NO: 9 | CTTCTCAGGAGCCTCTTCA |
| mTSLP R | SEQ ID NO: 10 | AGCCAGGGATAGGATTGA |
| mST-2 F | SEQ ID NO: 11 | GTGACACCTTACAAAACCCG |
| mST-2 R | SEQ ID NO: 12 | TCAAGAACGTCGGGCAGAG |
| mGAPDH F | SEQ ID NO: 13 | ACGGCCGCATCTTCTTGTGCA |
| mGAPDH R | SEQ ID NO: 14 | AATGGCAGCCCTGGTGACCA |
| hIL33 F | SEQ ID NO: 15 | GTGACGGTGTTGATGGTAAGA |
| hIL33 R | SEQ ID NO: 16 | CTCCACAGAGTGTTCCTTGTT |
| hST-2 F | SEQ ID NO: 17 | AACGAGTTACCAATACTTGCTC |
| hST-R | SEQ ID NO: 18 | CAGGCACTATTGCTTCTGGG |
| h18S F | SEQ ID NO: 19 | CTGAGAAACGGCTACCACATC |
| h18S R | SEQ ID NO: 20 | GCCTCGAAAGAGTCCTGTATTG |

### Microarray and Analysis.

The OneArray microarray service from Phalanx Biotech (Palo Alto, Calif.) was used in which 29,922 mouse genome probes and 1,880 experimental control probes were used for the array. Each sample was studied in triplicates. The following analyses of the microarray results were performed and provided with the service: (1) Rosetta profile error model calculation; (2) normalized intensities (excluding flagged and control data) with median scaling; (3) basic statistic plot and Pearson correlation coefficient; (4) pairwise ratio calculation; (5) principal component analysis; and (6) gene ontology analysis.

### Patient Samples.

BAL fluid cells were studied from asthmatic patients and disease control subjects. Asthma was diagnosed based on the presence of reversible airway obstruction, positive methacholine test results (PC20, <8 mg/mL), or both, per the Expert Panel Report 3 criteria. The clinical characteristics of the study subjects are shown in Table 4. Bronchoscopy, BAL, and endobronchial biopsy were performed, as described in Good et al. (Good J T, et al. Refractory asthma: importance of bronchoscopy to identify phenotypes and direct therapy. Chest 2012; 141:599-606). BAL fluid was processed immediately for flow cytometry. IL-33 was analyzed in BAL fluid by means of ELISA, according to the manufacturer's directions (R&D Systems).

TABLE 4
Clinical characteristics of human subjects

| Parameters | Asthma (N = 38) | Disease Controls (N = 18) |
|---|---|---|
| Diagnosis and comorbidities | 38 patients with asthma; | 7 patients with chronic cough |
| | co-morbidities: 30 patients | and concurrent allergic rhinitis |
| | with allergic rhinitis, 25 with | & GERD, 3 patients with |
| | chronic sinusitis, 29 with | bronchiectasis, 3 patients with |
| | GERD, 3 with bronchiectasis | MAC infection, 4 with chronic |
| | and 4 with aspiration | aspiration and 1 with COPD |
| Age | 54 ± 4 (56) | 56 ± 7 (58) |
| Mean ± SEM (median) | | |
| Sex (F/M) | 22/16 | 11/7 |
| BMI | 28 ± 3 (27) | 27 ± 3 (26) |
| FEV1 (%) | 69 ± 4 (72) | 82 ± 8 (76) |
| Reversibility | 16 ± 3 (12) | 5 ± 3 (6) |
| PC20 for methacholine | 2.9 ± 1 (1.58) | Not done |
| Blood eosinophils | 370 ± 80 (200) | 116 ± 25 (100) |
| Medications | ICS + LABA (32) | ICS + LABA (12) |
| | Systemic glucocorticoids (3) | ICS (3) |
| | ICS (2) | Montelukast (0) |
| | Omalizumab (6) | Inhaled anticholinergics (3) |
| | Montelukast (8) | SABA (18) |
| | SABA (38) | |

Abbreviations:
BMI: body mass index;
COPD: chronic obstructive pulmonary disease;
FEV1: forced expiratory volume in 1 second;
GERD: gastroesophageal reflux disease;
ICS: inhaled corticosteroid;
LABA: long-acting bronchodilator;
MAC: Mycobacterium avium complex;
PC20: provocation concentration inducing a 20% drop in FEV1;
SABA: short-acting bronchodilator

### Statistical Analyses.

Data are presented as means 6 SEMs. For comparison of airway hyperreactivity, 2-way ANOVA for repeated measures with a Bonferroni post hoc test was used. For pairwise comparisons, a Student t test was used. Data from human subjects were analyzed by using nonparametric tests (Mann-Whitney U test and Kruskal-Wallis test). A P value less than 0.05 was considered significant.

### Results

Repetitive Allergen Exposure Induces Asthma that Persists Longer than 6 Months after Cessation of Allergen Exposure

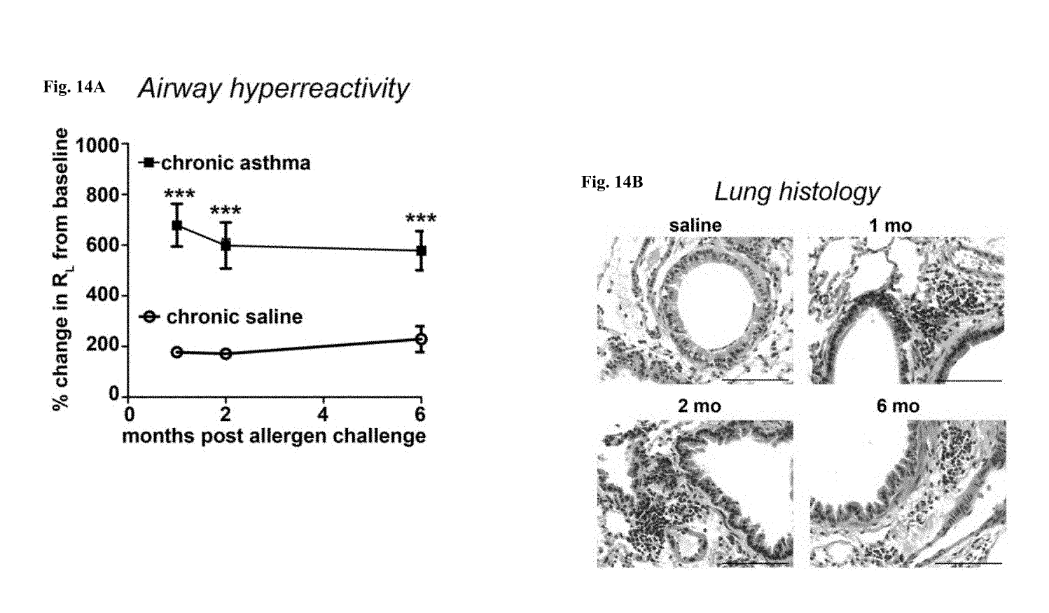

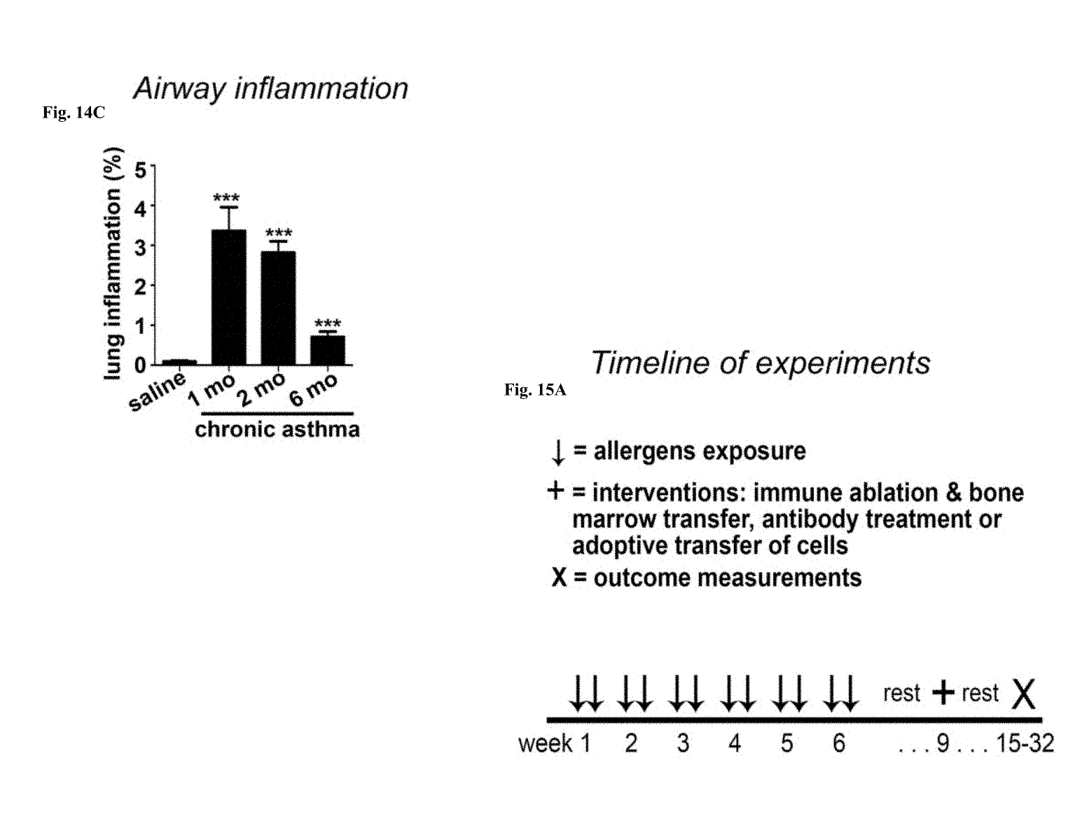

To establish experimental asthma in mice, 3 representative allergens were administered intranasally (ie, dust mite, ragweed, and Aspergillus species) without adjuvant twice a week for 6 weeks, as described in Goplen et al. (Goplen N, et al. Combined sensitization of mice to extracts of dust mite, ragweed, and Aspergillus species breaks through tolerance and establishes chronic features of asthma. J Allergy Clin Immunol 2009; 123:925-32.e11). In this model airway hyperreactivity persisted for longer than 6 months (FIG. 14A) after cessation of allergen exposure. Airway inflammation (FIGS. 14B and 14C), and epithelial and peribronchial smooth muscle (FIG. 14D) hypertrophy peaked 1 to 2 months after allergen cessation but remained significantly increased after 6 months. The previously reported increase in mucin and mast cell levels also persisted in patients with chronic asthma (see FIG. 20A-20D).

### T Cells Contribute to the Severity but not Persistence of Asthma

To examine the mechanism of asthma persistence, the following timeline was designed for various interventions (FIG. 15A). Previous studies have demonstrated resolution of acute asthma within 10 to 14 days after allergen exposure (Duez C, et al. Fas deficiency delays the resolution of airway hyperresponsiveness after allergen sensitization and challenge. J Allergy Clin Immunol 2001; 108:547-56; Haworth O, et al. NK cells are effectors for resolvin E1 in the timely resolution of allergic airway inflammation. J Immunol 2011; 186: 6129-35: Leech M D, et al. Resolution of Der p1-induced allergic airway inflammation is dependent on CD41CD251Foxp31 regulatory cells. J Immunol 2007; 179:7050-8). To demonstrate the persistence of asthma, outcomes 3 weeks after the last allergen exposure was studied. Nearly all interventions were carried out in week 10. Lethal irradiation of mice was done at the end of week 9. Lung inflammation and airway hyperreactivity were measured 6 weeks later at week 15 to provide sufficient time for marrow engraftment and restoration of the immune response. The contribution of T cells in asthma maintenance using 3 different approaches was examined. First, mice with chronic asthma were treated with an anti-CD3ε neutralizing antibody or an isotype control antibody at week 10. This treatment reduced T-cell counts by 91% in the spleen (see FIG. 20A) and inhibited airway inflammation, as determined by means of quantification of lung histology, by 70% (see FIG. 20B). However, the anti-CD3ε antibody did not alter airway hyperreactivity (FIG. 15B). To some extent, these results are similar to those reported by Doherty et al. (Doherty T A, et al. CD41 cells are required for chronic eosinophilic lung inflammation but not airway remodeling. Am J Physiol Lung Cell Mol Physiol 2009; 296:L229-35) who demonstrated that anti-CD4 T-cell depletion inhibited airway inflammation but not smooth muscle mass, mucus metaplasia, or airway fibrosis in a mouse model of chronic allergen exposure.

Second, the inventors adoptively transferred splenic CD4 T cells from chronic saline-treated mice or from the chronic asthma model (isolated in week 10) to naive mice and subsequently challenged the recipient mice intranasally with the sensitizing allergens on 3 consecutive days. Adoptively transferred CD4 T cells from mice with chronic asthma potently induced airway hyperreactivity in naive mice 6 days after the transfer (FIG. 15C), thus confirming the ability of CD4 T cells from mice with chronic asthma to induce acute asthma in naive mice. However, airway hyperreactivity did not persist when examined on day 21.

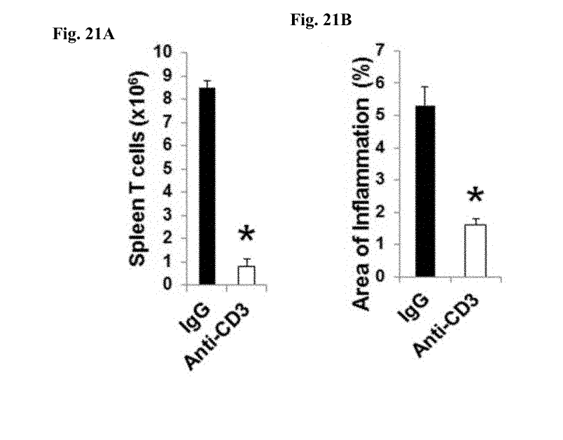

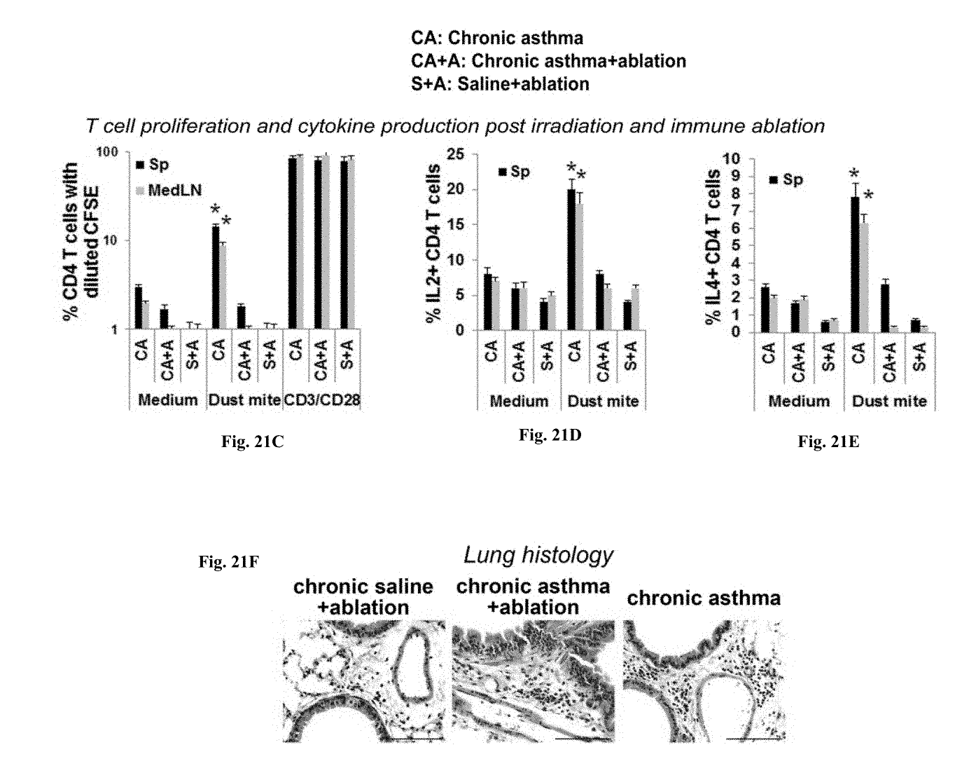

Third, mice with chronic asthma were subjected to lethal irradiation at the end of week 9 and transplanted bone marrow from naive mice to sustain mouse viability. Splenic and mediastinal lymph node-derived T cells collected 6 weeks after irradiation proliferated in response to anti-CD3/CD28 antibodies but did not respond to the recall antigens dust mite (see FIG. 21C), ragweed, and Aspergillus species (not shown). This indicates elimination of sensitized T cells in irradiated mice. In addition, antigen stimulation did not increase the number of cytokine producing cells over baseline (medium stimulation) after immune ablation. In contrast, an increased number of T cells from the control mice with chronic asthma (not subjected to lethal irradiation) produced IL-2 (see FIG. 21D) and IL-4 (see FIG. 21E) after dust mite stimulation. The results indicate that lethal irradiation has eliminated antigen-specific T cells. This effect of lethal irradiation will henceforth be referred to as immune ablation.

Airway inflammation was monitored at weeks 11, 13, and 15 (2, 4, and 6 weeks after immune ablation). Consistent with the absence of allergen-specific T cells, airway inflammation was nearly absent at week 11 but gradually increased, reaching statistical significance at week 15 (FIG. 15D, and see FIG. 21F). Epithelial and smooth muscle hypertrophy (FIG. 15E) and airway hyperreactivity (FIG. 15F) persisted after immune ablation, although the degree of airway hyperreactivity was reduced. These results suggest that persistence of airway hyperreactivity and remodeling did not require the presence of sensitized T cells. However, the magnitude of airway hyperreactivity was amplified by sensitized T cells.

### Gene Expression Profile Shows Increased Levels of ILC2-Promoting Molecules in Chronic Asthma

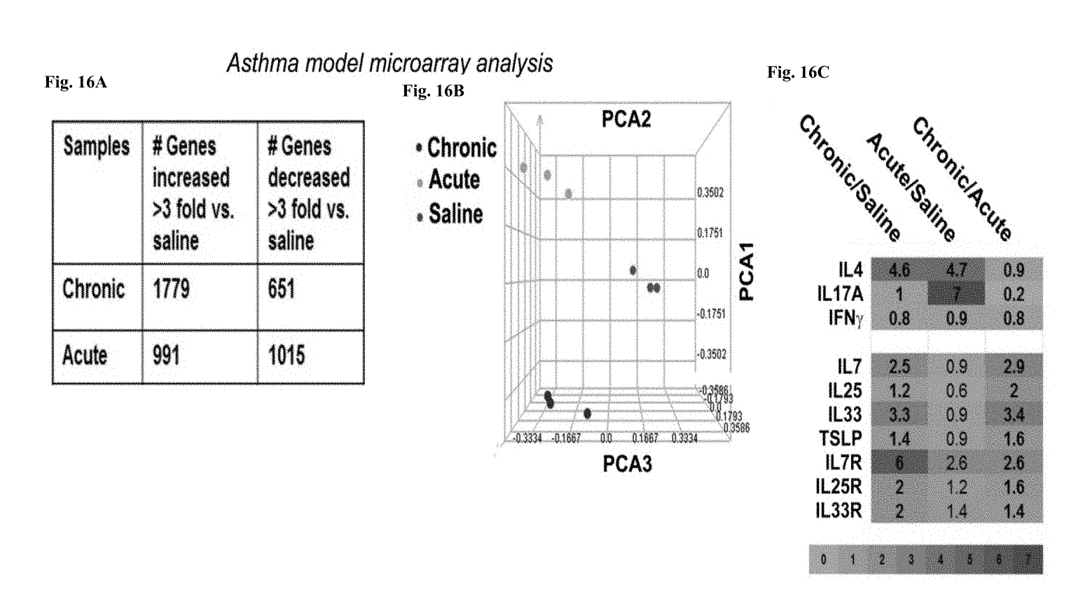

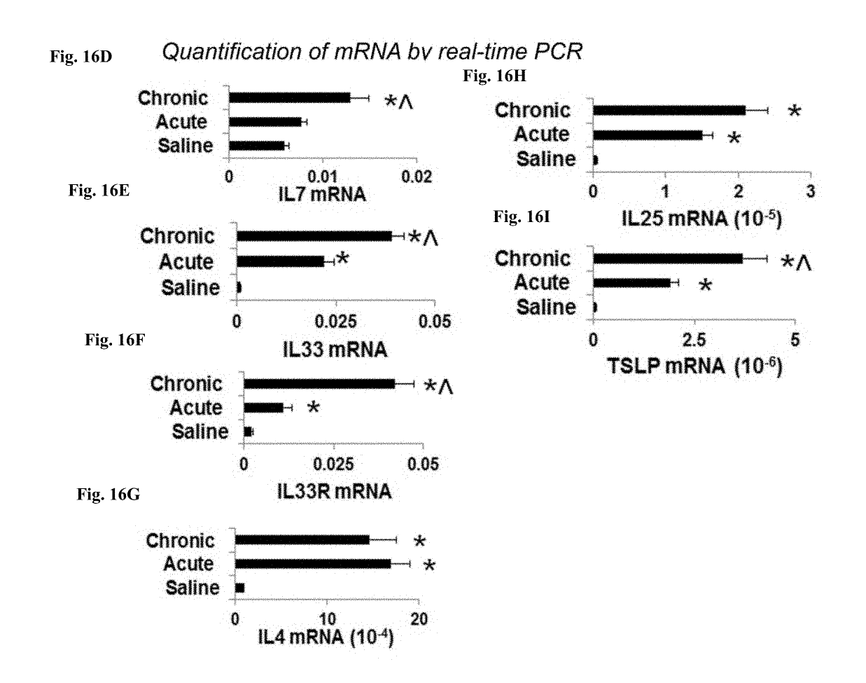

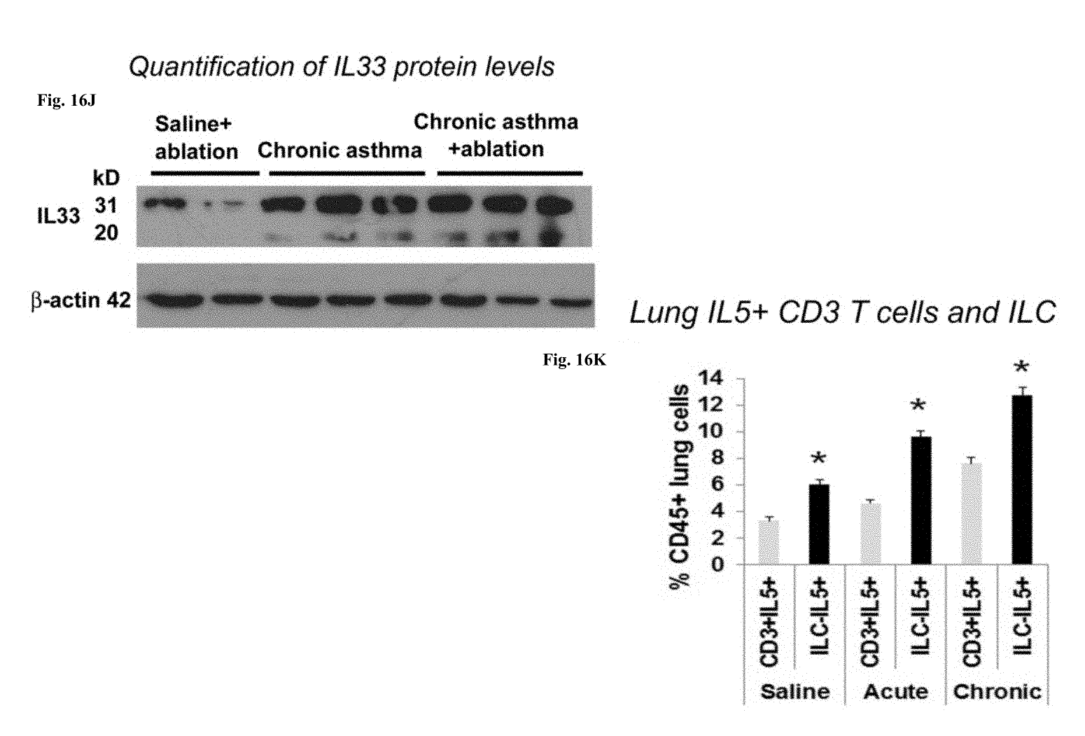

Gene expression in lung tissue from the chronic asthma model was compared using a microarray with 2 different controls, acute asthma and saline controls (Goplen N, et al. Combined sensitization of mice to extracts of dust mite, ragweed, and Aspergillus species breaks through tolerance and establishes chronic features of asthma. J Allergy Clin Immunol 2009; 123:925-32.e11). Approximately 1000 genes were observed upregulated and downregulated in the acute model by using a cutoff of a 3-fold difference with the saline control and nearly 3 times more genes upregulated than downregulated in mice with chronic asthma compared with saline control animals (FIG. 16A). The principal component analysis (FIG. 16B)-derived positions of acute and chronic asthma do not overlap, indicating a qualitative difference in gene expression. Because sensitized T cells were not essential for sustaining asthma, ILC2-inducing cytokines were analyzed (Saenz S A, et al. IL25 elicits a multipotent progenitor cell population that promotes TH2 cytokine responses. Nature 2010; 464:1362-6; Kim H Y, et al. Innate lymphoid cells responding to IL-33 mediate airway hyperreactivity independently of adaptive immunity. J Allergy Clin Immunol 2012; 129:216-27, e211-6; Wong S H, et al. Transcription factor RORalpha is critical for nuocyte development. Nat Immunol 2012; 13:229-36). Increased expression of IL-7, IL-33, and their receptors was observed in the setting of chronic asthma (FIG. 16C), which was confirmed by using real-time PCR (FIG. 16D-16F). Levels of IL-4, but not IFN-γ, were increased in both the acute and chronic asthma groups relative to those seen in the saline control group (FIG. 16C). IL-4 expression was confirmed by using real-time PCR (FIG. 16G). In agreement with a recent study that demonstrated a central role for IL-33 compared with IL-25 and thymic stromal lymphopoietin in dust mite and peanut allergic sensitization (Chu D K, et al. IL-33, but not thymic stromal lymphopoietin or IL-25, is central to mite and peanut allergic sensitization. J Allergy Clin Immunol 2013; 131:187-200, e181-8). IL-25 and thymic stromal lymphopoietin expression, determined by using real-time PCR, was 100- to 1000-fold lower than that of IL-33 (FIGS. 16H and 16I) in the acute and chronic asthma groups. IL-33 expression was confirmed in the airways after immune ablation using Western blotting (FIG. 16J). The larger 31-kDa form of IL-33 undergoes proteolytic cleavage to produce a more active 20-kDa form (Lefrancais E, et al. IL-33 is processed into mature bioactive forms by neutrophil elastase and cathepsin G. Proc Natl Acad Sci USA 2012; 109:1673-8). The 20-kDa form was present in the chronic asthma group but not in the saline control group and persisted after immune ablation.

### Chronic Asthma is Characterized by Increased ILC2 Numbers

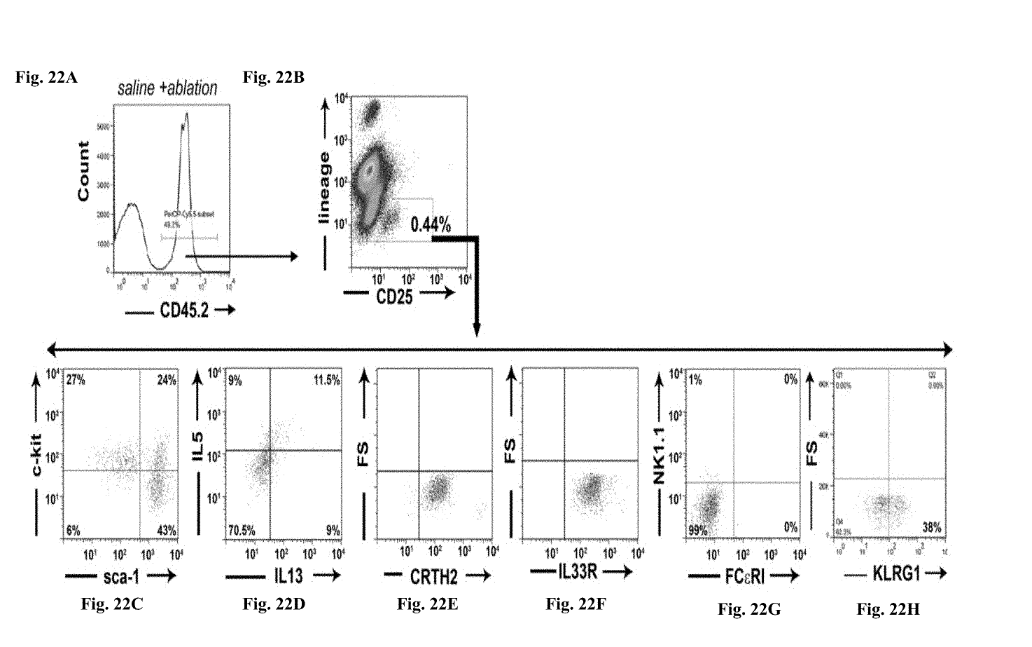

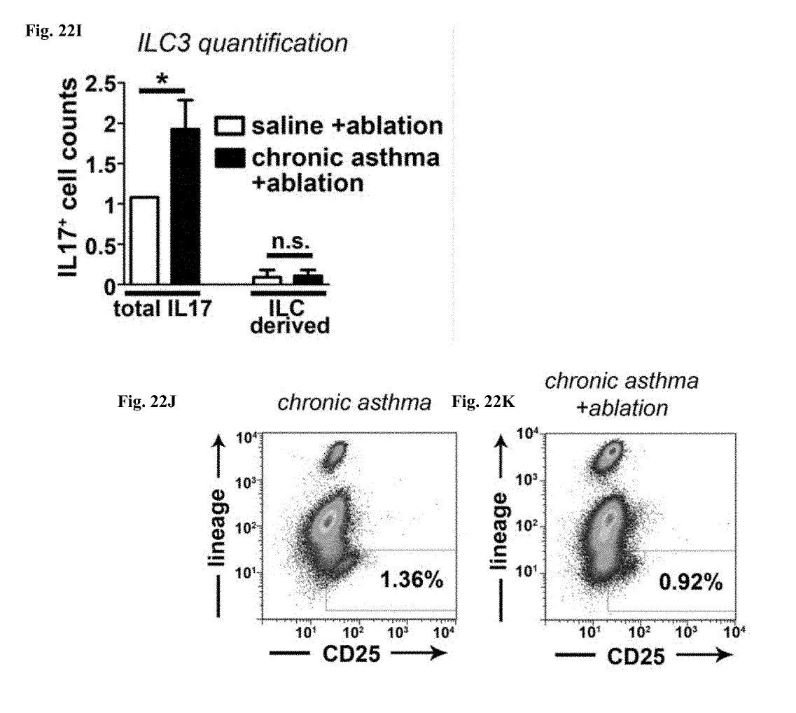

Previous studies have demonstrated a crucial role for ILC2s in the development of lung inflammation from Alternaria species (Doherty T A, et al. 2012) and papain (Halim T Y, et al. Lung natural helper cells are a critical source of Th2 cell-type cytokines in protease allergen-induced airway inflammation. Immunity 2012; 36:451-63) airway hyperreactivity caused by influenza (Chang Y J, et al. Innate lymphoid cells mediate influenza-induced airway hyper-reactivity independently of adaptive immunity. Nat Immunol 2011; 12:631-8), and allergic sensitization from dust mite and peanut (Chu D K, et al. IL-33, but not thymic stromal lymphopoietin or IL-25, is central to mite and peanut allergic sensitization. J Allergy Clin Immunol 2013; 131:187-200, e181-8). The inventors microarray data suggest a role for ILC2s in the maintenance of chronic asthma. ILC2s was studied in the lung digest, as described previously (Neill D R, et al. Nuocytes represent a new innate effector leukocyte that mediates type-2 immunity. Nature 2010; 464:1367-70; Saenz S A, et al. IL25 elicits a multipotent progenitor cell population that promotes T(H)2 cytokine responses. Nature 2010; 464:1362-6; Halim T Y, et al. Lung natural helper cells are a critical source of Th2 cell-type cytokines in protease allergen-induced airway inflammation. Immunity 2012; 36:451-63). First, the inventors gated for live CD45+ cells (see FIG. 22A-22H). Next, the inventors identified lineage (CD3, Ly-6G/Ly-6C, CD11b, B220, TER-119)-negative but CD25+ cells. This lin-CD25+ population was positive for the ILC2 markers c-Kit, Sca-1, chemoattractant receptor-homologous molecule expressed on TH2 lymphocytes (CRTH2), IL-33 receptor (IL-33R), and killer cell lectin-like receptor G superfamily, member 1 (KLRG1) and negative for NK1.1 and FcεRI. About one fourth of these lin-CD25+CRTH2+IL-33R+ ILC2s were positive for IL-5 and IL-13 in nonimmunized mice. This is in agreement with previous reports that showed low basal expression of IL-5 and IL-13 and increased expression on stimulation (Moro K, et al. Innate production of T(H)2 cytokines by adipose tissue-associated c-Kit(1)Sca-1(1) lymphoid cells. Nature 2010; 463:540-4; Neill D R, et al. Nuocytes represent a new innate effector leukocyte that mediates type-2 immunity. Nature 2010; 464:1367-70; Saenz S A, et al. IL25 elicits a multipotent progenitor cell population that promotes T(H)2 cytokine responses. Nature 2010; 464:1362-6). These cells were negative for IL-17 (see FIG. 22I). The frequency of IL-5+ innate lymphoid cells (ILCs) was consistently higher than that of IL-5+ T cells in all study groups (FIG. 16K).

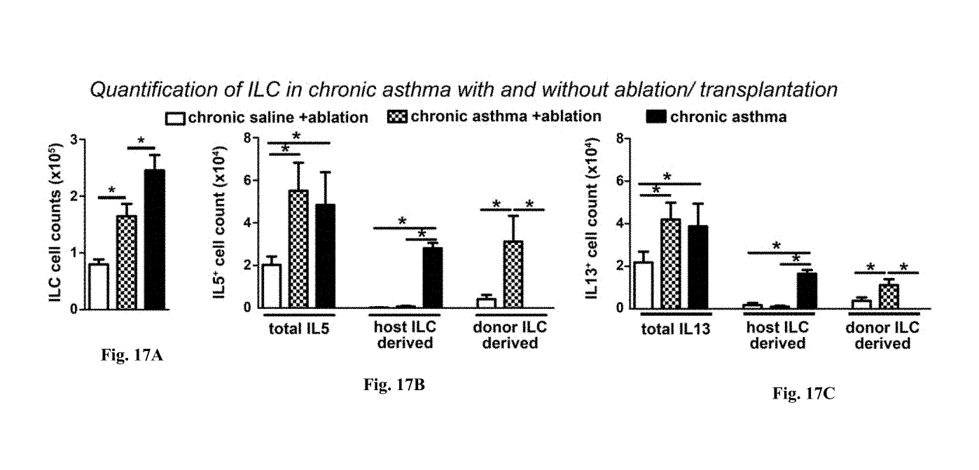

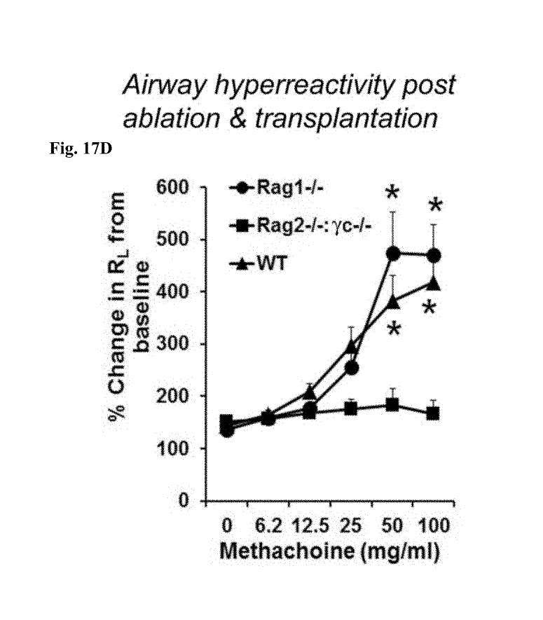

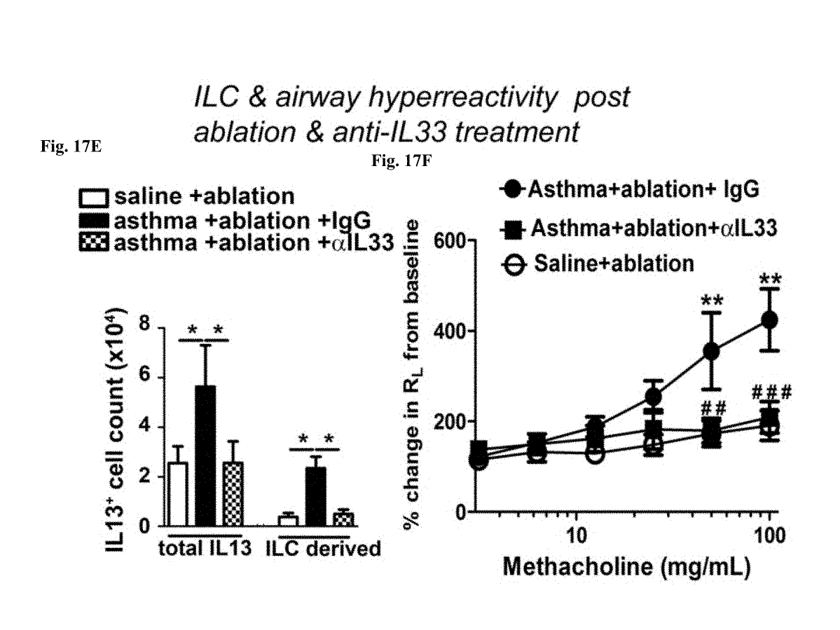

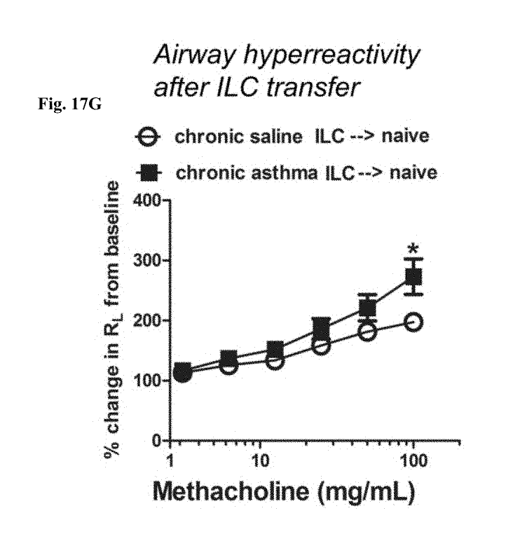

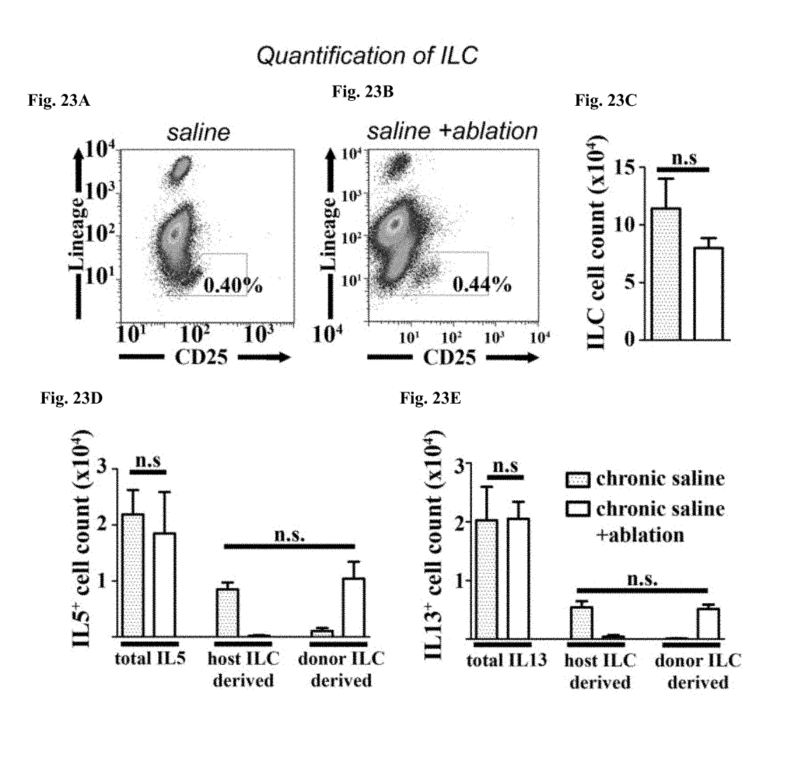

The total lin-CD25+ ILC population was increased in the chronic asthma versus saline control groups (FIG. 17A, and see FIGS. 22B, 22J, and 22K). After immune ablation and naive bone marrow transplantation, the ILC population decreased but remained increased compared with that in the saline control group. IL-5+ ILC2s constituted 61% of all IL-5+ lung cells in the chronic asthma group (FIG. 17B). Irradiation caused a near-complete elimination of host-originated IL-5+ and IL-13+ ILC2s in both the chronic asthma (FIGS. 17B and 17C) and saline control (see FIGS. 23D and 23E) groups. This was replaced by donor derived ILCs. Interestingly, the number of donor-derived ILCs increased 3-fold in recipients of marrow from the chronic asthma group compared with that in the saline control group, suggesting a post-transfer expansion likely caused by established increased IL-33 production present in the chronic asthma group.

### ILC2s but not Antigen-Specific T Cells are Essential for the Persistence of Asthma

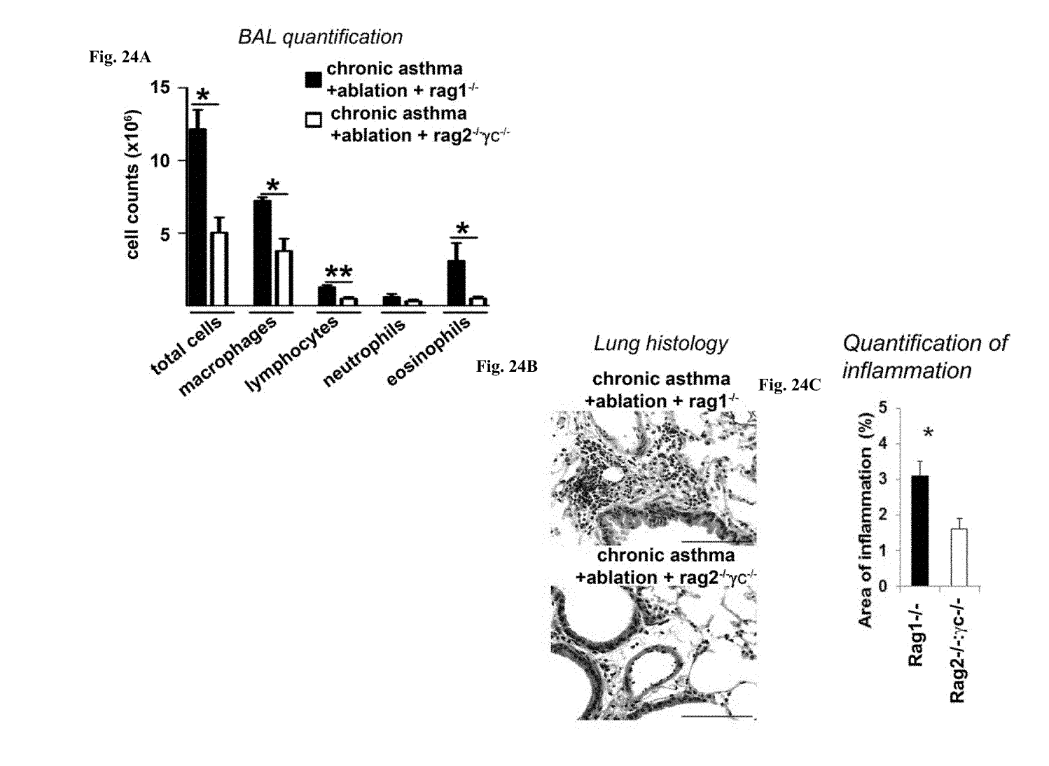

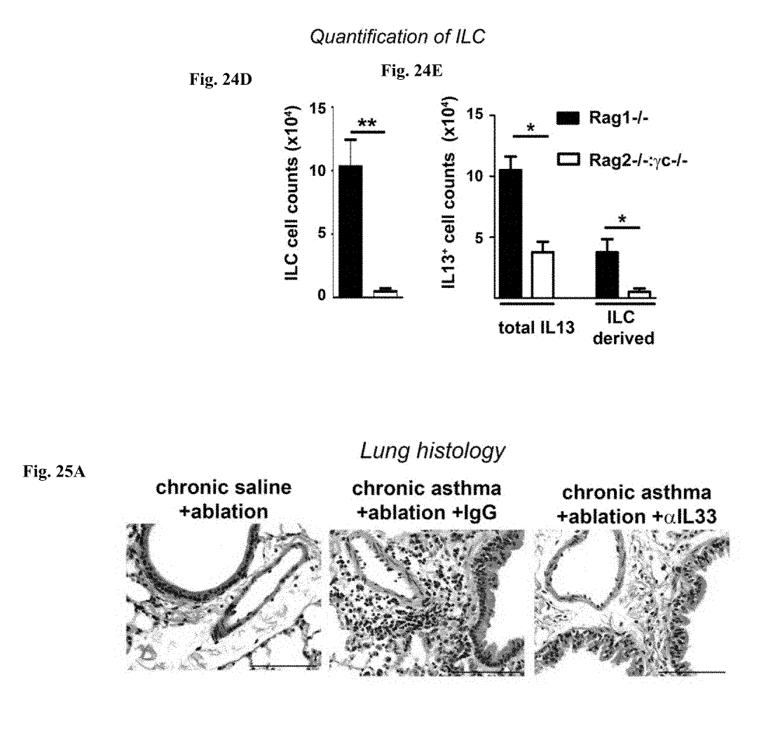

To determine the role of ILC2s in the persistence of asthma, bone marrow was transferred from recombination-activating gene (Rag1)−/−, Rag2−/− and common gc chain of the IL-2 receptor (yc)−/−, and wild-type mice to irradiated mice with chronic asthma. Rag1−/− mice are deficient in T and B cells but have normal ILC numbers, whereas Rag2−/−: γc−/− mice are additionally deficient in ILCs (Halim T Y, et al. 2012). Mice that received marrow from Rag1−/− and wild-type mice maintained airway hyperreactivity (FIG. 17D). In contrast, mice that received marrow from Rag2−/−: γc−/− mice lost airway hyperreactivity. Airway inflammation and increased inflammatory cells in bronchoalveolar lavage (BAL) fluid persisted in recipients of Rag1−/− but not Rag2−/−: γc−/− marrow (see FIG. 24A-24C). The total number of ILCs (lin-CD25+) was comparable between recipients of Rag1−/− marrow (see FIG. 24D) and naive marrow (FIG. 17A). The ILC population was nearly absent in recipients of Rag2−/−: γc−/− marrow (see FIG. 24D). Despite the absence of ILCs and T cells, the recipients of Rag2−/−: γc−/− marrow had a significant number of IL-13+ cells in the lungs (see FIG. 24E), although this was not sufficient to sustain asthma. This could be due to the difference in the quantity of IL-13 or other cytokines/factors made by ILC2s. The lack of asthma in Rag2−/−: γc−/− marrow recipient mice eliminates the possibility that radio-resistant antigen-specific T cells or ILC2s that might have persisted after irradiation were responsible for the sustenance of asthma in mice with chronic asthma that received naive marrow (FIG. 15D-15F).

### IL-33 Blockade Abolishes Airway Hyperreactivity, Inflammation, and IL-5/IL-13-Producing Cells

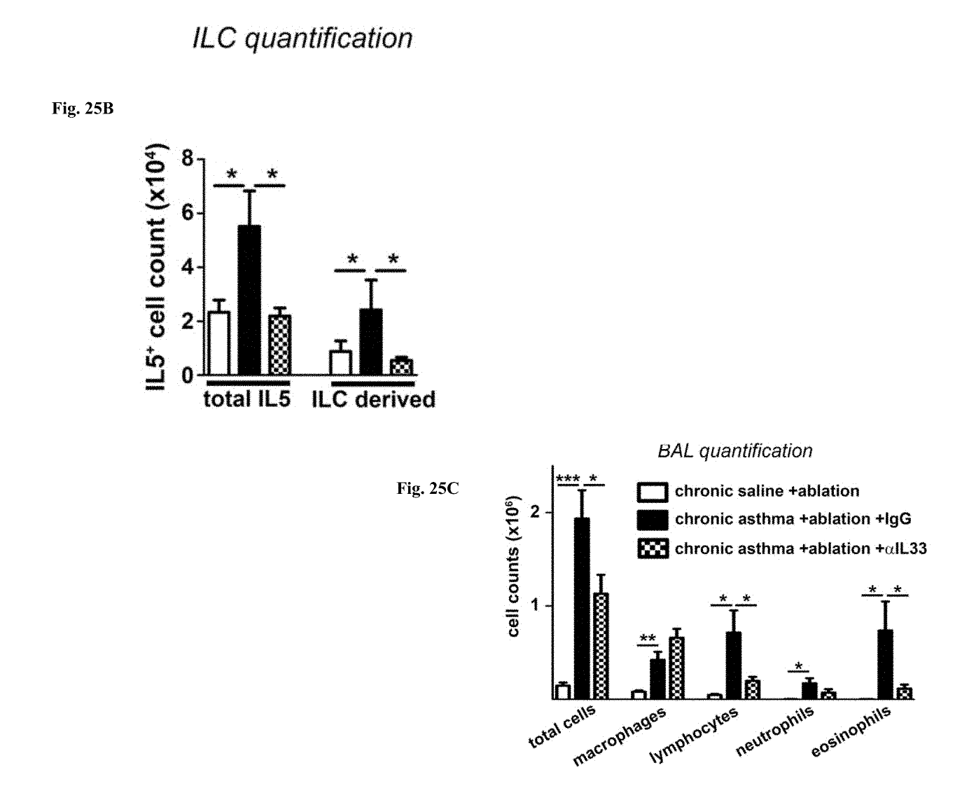

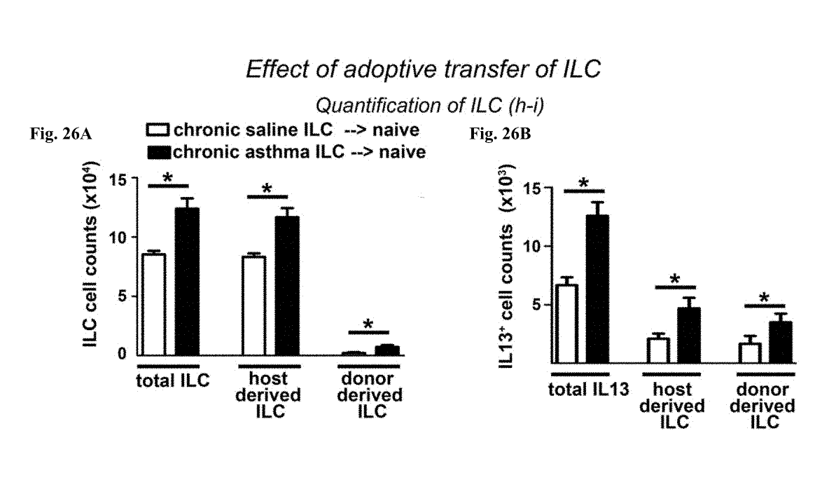

Persistent production of IL-33 after immune ablation (FIG. 16J) suggests that IL-33-driven ILC2s might be critical for the persistence of asthma. To test this, 3 doses of an anti-IL-33 antibody or goat IgG were administered to immune-ablated mice with chronic asthma 1 week before the outcome measures in week 15. Anti-IL-33 treatment reduced the number of total lung ILCs (CD45+ lin-CD25+), IL-5+ ILC2s, total IL-5+ lung cells (see FIG. 25B), IL-13+ ILC2s, and total IL-13+ lung cells to normal non-asthmatic levels (FIG. 17E). This was associated with a complete resolution of airway hyperreactivity (FIG. 17F) and a significant reduction in airway inflammation (see FIG. 25A). IL-33 blockade also reduced total cell, lymphocyte, and eosinophil numbers in BAL fluid (see FIG. 25C).

### Adoptive Transfer of ILCs Induces Sustained Airway Hyperreactivity

The foregoing experiments demonstrated that airway hyperreactivity could not be sustained in the absence of IL-33 or ILC2s. Lung CD45+ lin-CD25+ cells were sorted from the chronic asthma and saline control groups (both CD45.11) and adoptively transferred (2×10⁵ cells) to naive congenic CD45.2+ mice to demonstrate whether activated ILC2s from asthmatic mice were sufficient to sustain airway hyperreactivity. Donor-derived ILCs were detected in the recipient lung 21 days after transfer (see FIG. 26). In contrast to adoptive transfer of CD4 T cells from mice with chronic asthma, significant airway hyperreactivity 21 days after adoptive transfer was observed in recipients of ILCs from mice with chronic asthma (FIG. 17G).

### Airway Epithelial Cells Establish a Positive Feedback Circuit Through IL-33 and ILC2s

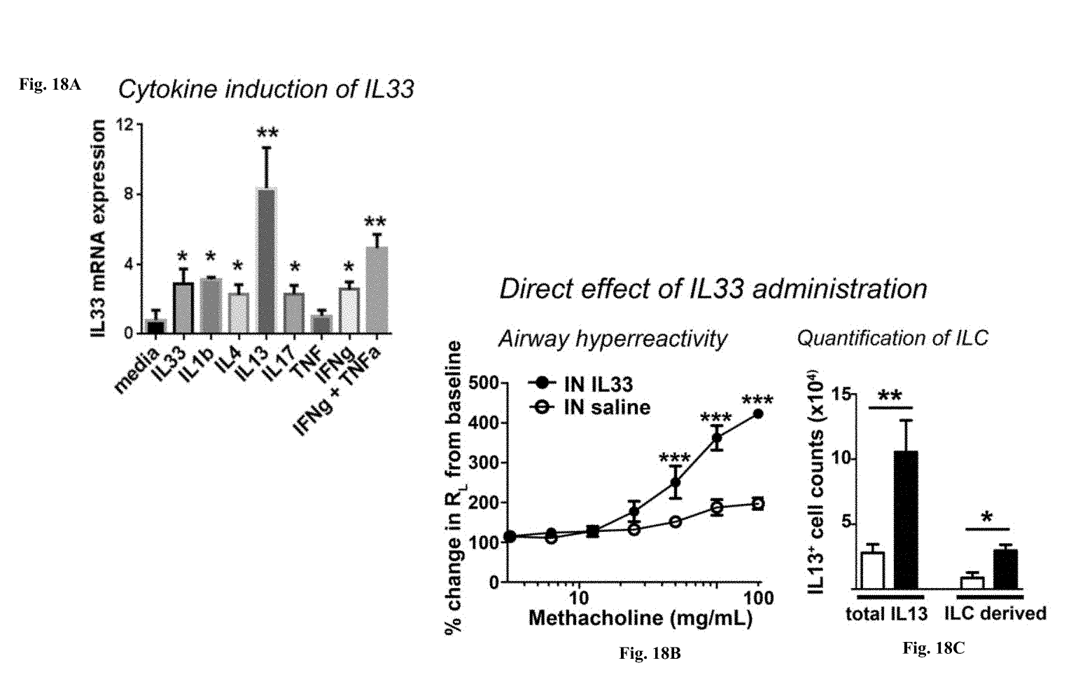

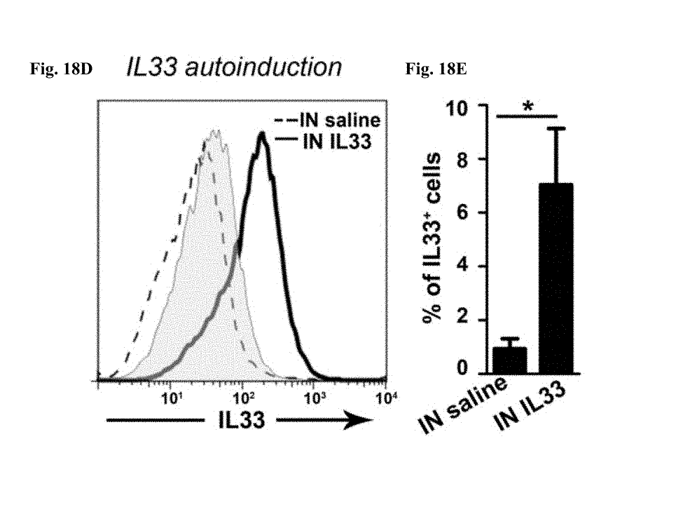

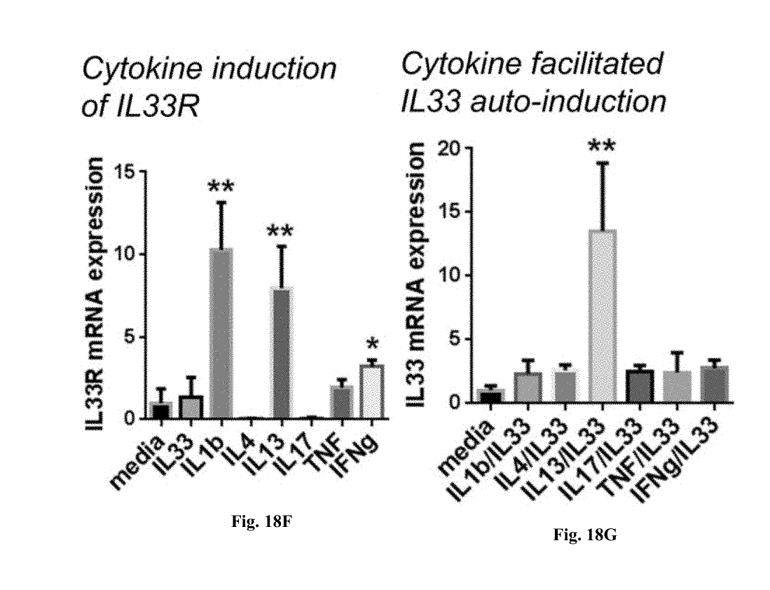

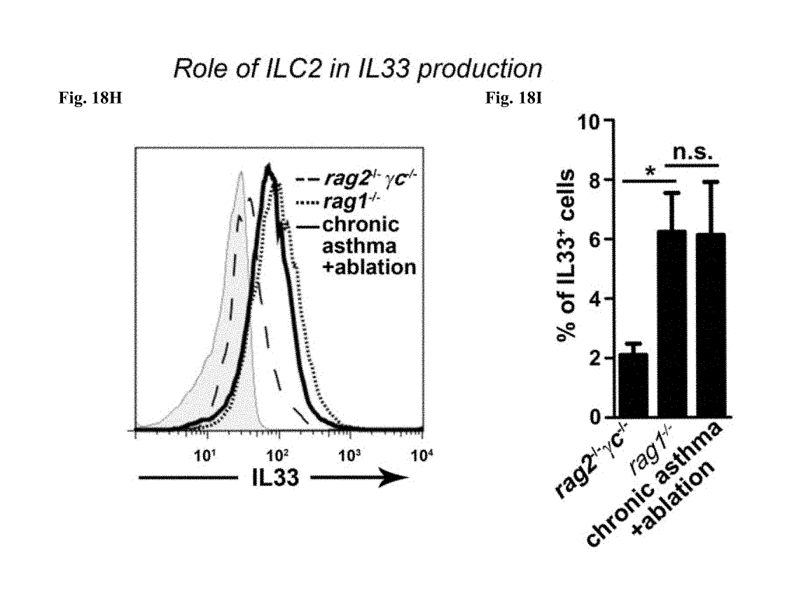

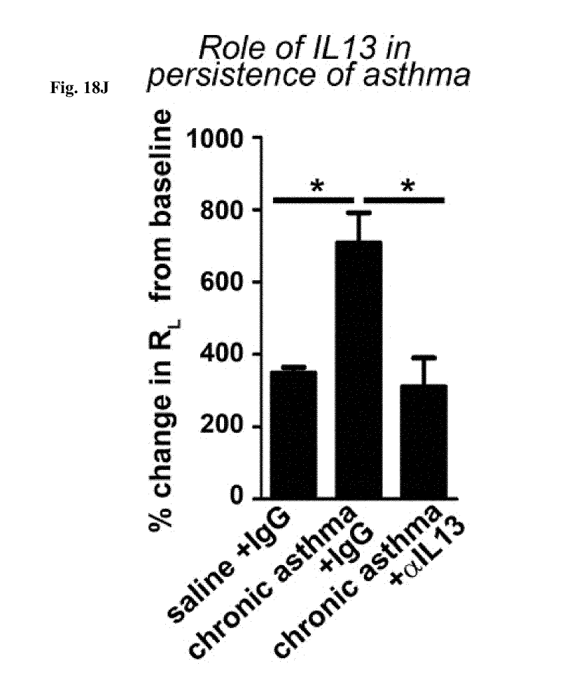

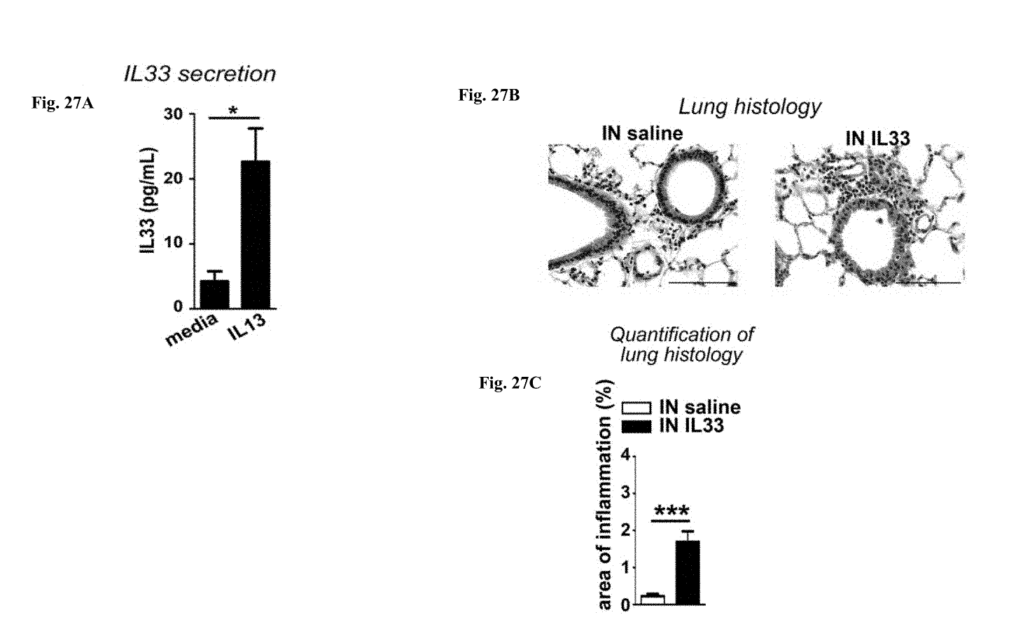

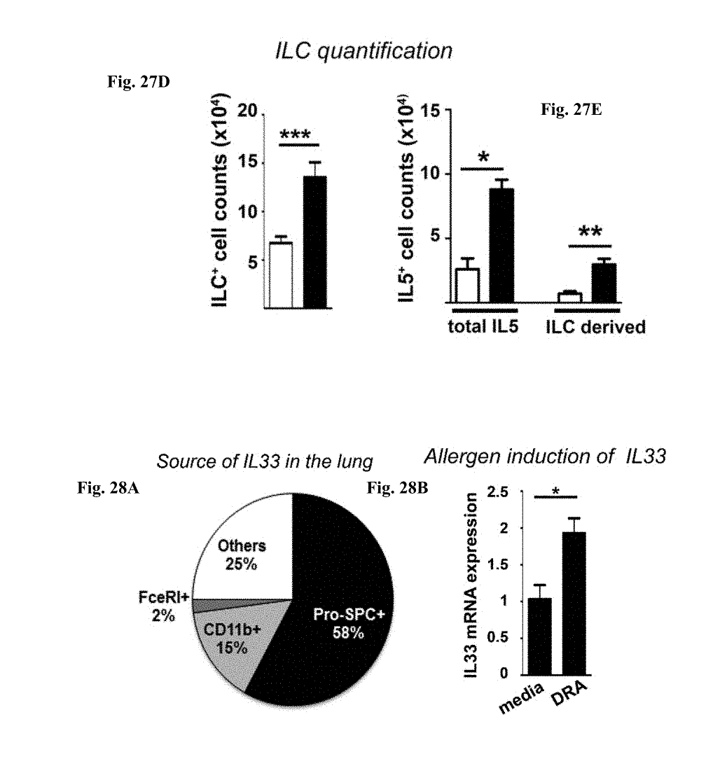

Self-sustenance of biological processes can be facilitated by the development of a positive feedback circuit or circuits. This putative mechanism was tested by examining the effect of IL-13, a major product of ILC2s, on epithelial production of IL-33 in the human lung epithelial cell line A549. IL-13 was the most potent inducer of IL-33 mRNA compared with IL-33, IL-1b, IL-4, IL-17, TNF, and IFN-γ (FIG. 18A). IL-13 also stimulated secretion of IL-33 (see FIG. 27A). Similar results were observed with the BEAS2B human bronchial epithelial cell line (not shown). These results suggest that IL-13 can induce epithelial production of IL-33, establishing a positive feedback circuit using ILC2s.

### IL-33 Autoinduction Represents Another Positive Feedback Circuit in the Setting of Chronic Asthma

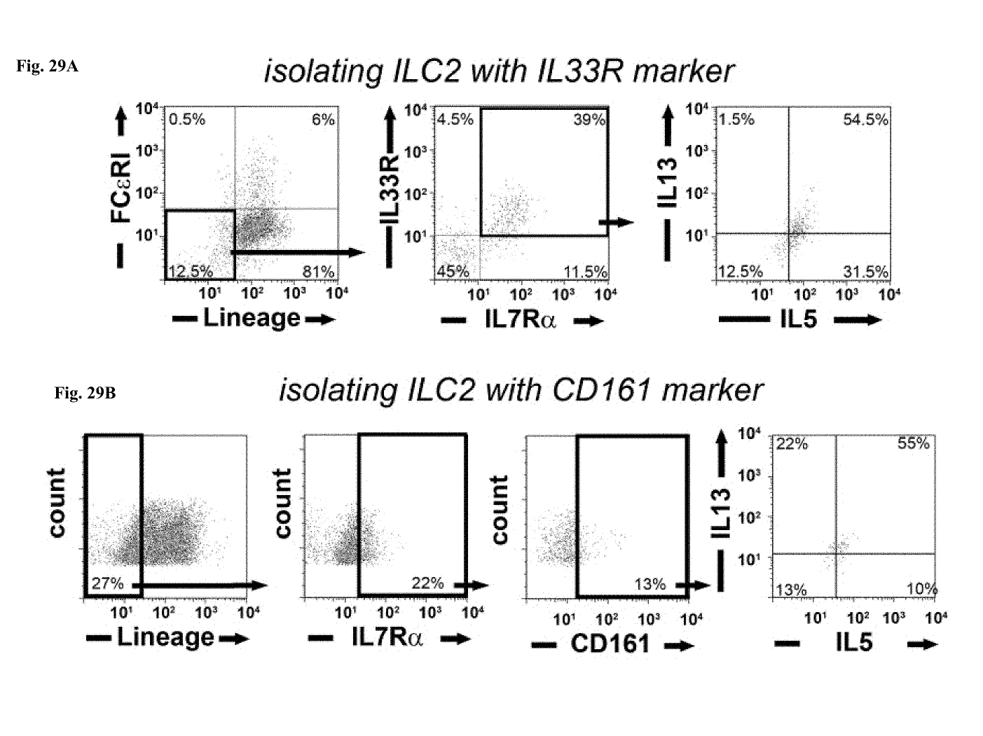

Previous studies have shown that IL-33 induces acute asthma in mice when examined 24 hours after the ultimate dose (Bartemes K R, et al. IL-33-responsive lineage-CD251 CD44(hi) lymphoid cells mediate innate type 2 immunity and allergic inflammation in the lungs. J Immunol 2012; 88:1503-13; Kondo Y, et al. Administration of IL-33 induces airway hyperresponsiveness and goblet cell hyperplasia in the lungs in the absence of adaptive immune system. Int Immunol 2008; 20:791-800; Kurowska-Stolarska M, et al. IL-33 induces antigen-specific IL-51 T cells and promotes allergic-induced airway inflammation independent of IL-4. J Immunol 2008; 181:4780-90; Hardman C S, et al. IL-33 citrine reporter mice reveal the temporal and spatial expression of IL-33 during allergic lung inflammation. Eur J Immunol 2013; 43:488-98). To extend these findings to chronic asthma, the inventors examined the persistence of asthma 15 days after intranasal IL-33 administration. IL-33 induced airway hyperreactivity (FIG. 18B) and mild airway inflammation (see FIGS. 27B and 27C) that persisted for 2 weeks. IL-33 also increased the number of lung IL-131 ILC2s (FIG. 18C), the total number of IL-51 and IL-131 lung cells, and IL-51 ILC2 numbers (see FIGS. 27D and 27E). These experiments suggest that IL-33 induces a sustained effect in the airways. To address the mechanism for this sustained effect, IL-33 autoinduction was investigated. Hardman et al. initially described IL-33 autoinduction 24 hours after intranasal delivery of IL-33. To extend these findings, persistent IL-33 production was observed (FIGS. 18D and 18E) in pro-surfactant protein C-positive epithelial cells (see FIG. 28A) and CD11b+ hematopoietic cells 2 weeks after intranasal IL-33 administration. These results are in agreement with the data from an IL-33 reporter mouse model (Hardman C S, et al. IL-33 citrine reporter mice reveal the temporal and spatial expression of IL-33 during allergic lung inflammation. Eur J Immunol 2013; 43:488-98) and the inventors previous in vitro experiments (FIG. 18A) indicating that IL-33 was capable of autoinduction.

### IL-13 Generates a Feed-Forward Mechanism for IL-33 Autoinduction

Airway epithelial cells express low basal levels of mRNA for IL-33R (Yagami A, et al. IL-33 mediates inflammatory responses in human lung tissue cells. J Immunol 2010; 185:5743-50), thus permitting IL-33 autoinduction. IL-13 and IL-13, but not IL-4 or IL-17, strongly induced IL-33R (FIG. 18F). Next, the inventors investigated whether preincubation with the IL-33-inducing cytokines facilitated IL-33 autoinduction. Only IL-13, but not IL-13, IL-4, or IL-17, synergistically enhanced IL-33 autoinduction (FIG. 18G). Although this synergy was modest, the result suggests that IL-13 was mechanistically connected with IL-33 autoinduction. Given that IL-33 stimulates ILC2s, which subsequently produce IL-13 capable of stimulating additional IL-33 release, the result suggests an IL-33-driven feed-forward mechanism. Although IL-113 was a strong inducer of IL-33R, it was unable to facilitate IL-33 autoinduction. This is likely due to competition for the shared IL-1 receptor accessory protein (IL1RAcP) between IL-33R and IL-10. To confirm a role for our triple-allergen cocktail used in the induction of the chronic asthma model, we tested their effect on IL-33 production in vitro was tested. Dust mite, ragweed, and Aspergillus species directly stimulated IL-33 mRNA transcription 24 hours after exposure in A549 epithelial cells (see FIG. 28B). This is in agreement with direct induction of IL-33 by other allergens in previous studies (Bartemes K R, et al. IL-33-responsive lineage-CD251 CD44(hi) lymphoid cells mediate innate type 2 immunity and allergic inflammation in the lungs. J Immunol 2012; 88:1503-13; Doherty T A, et al. STAT6 regulates natural helper cell proliferation during lung inflammation initiated by Alternaria. Am J Physiol Lung Cell Mol Physiol 2012; 303:L577-88; Hardman C S, et al. IL-33 citrine reporter mice reveal the temporal and spatial expression of IL-33 during allergic lung inflammation. Eur J Immunol 2013; 43:488-98). IL-33 production persisted equally in recipients of ILC-sufficient naive and Rag1−/− bone marrow but not ILC^{deficient} Rag2−/−: γc−/− bone marrow (FIGS. 18H and 18I). This suggests that ILC2s were essential for sustained IL-33 production in vivo during chronic asthma.

### IL-13 is Important for the Persistence of Asthma

The inventors previously reported that a single administration of an anti-IL-13 antibody inhibited acute but not chronic asthma (Goplen N, et al. Combined sensitization of mice to extracts of dust mite, ragweed, and Aspergillus species breaks through tolerance and establishes chronic features of asthma. J Allergy Clin Immunol 2009; 123:925-32.e11). Because this study uncovered that IL-13 is likely to be involved in establishing a positive feedback mechanism, the inventors reasoned that repetitive IL-13 blockade would be required to override the feedback mechanism. To test this, 3 doses of an anti-IL-13 antibody were administered intraperitoneally to mice with chronic asthma in week 10. The airway hyperreactivity resolved 3 days after the anti-IL-13 antibody treatment (FIG. 18J).

### IL-33 and ILC2 Levels are Increased in Human Asthma

Next, the clinical relevance of IL-33 and ILC2s was examined. IL-33 levels were significantly increased in BAL fluid from asthmatic patients compared with disease control subjects (median, 560 pg/mL in asthmatic patients vs 295 pg/mL in disease control subjects; FIG. 19A). The demographic and clinical characteristics of the study subjects are provided in Table 4. BAL fluid IL-33 levels negatively correlated (r=0.60, P=0.0007) with airway flow volume (FIG. 19B) and Asthma Control Test scores, which reflect symptoms (r=0.56, P=0.04). In agreement with previous reports (Masamune A, et al. Nuclear expression of interleukin-33 in pancreatic stellate cells. Am J Physiol Gastrointest Liver Physiol 2010; 299:G821-32; Zhang L, et al. TLR-mediated induction of proallergic cytokine IL-33 in ocular mucosal epithelium. Int J Biochem Cell Biol 2011; 43:1383-91), IL-33 was localized to the nucleus of the basal cell layer of airway epithelium (FIG. 19C). Increased IL-33 levels predicted increased ILC2 numbers in the airways. Human ILC2s as lin-FcεRI-CD127+IL-33R+ cells were identified (see FIG. 29), as reported previously (Mj osberg J M, et al. Human IL-25- and IL-33-responsive type 2 innate lymphoid cells are defined by expression of CRTH2 and CD161. Nat Immunol 2011; 12:1055-62; Shaw J L, et al. IL-33-responsive innate lymphoid cells are an important source of IL-13 in chronic rhinosinusitis with nasal polyps. Am J Respir Crit Care Med 2013; 188:432-9). The majority of these cells expressed IL-5 and IL-13. The frequency of ILC2s in BAL fluid was significantly increased in asthmatic patients compared with that seen in disease control subjects (median, 1.2% in asthmatic patients vs 0.24% in disease control subjects; FIG. 19D). The number of IL-51 ILC2s was also increased in asthmatic patients (FIG. 19E). Human asthma is heterogeneous. Expectedly, there were high and low expressers of IL-33 and ILC2s. The results suggest that IL-33 and ILC2s are involved in a subgroup of asthmatic patients.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims. Each publication and reference cited herein is incorporated herein by reference in its entirety.

### REFERENCES

1. Robinson D S, Hamid Q, Ying S, Tsicopoulos A, Barkans J, Bentley A M, et al. Predominant TH2-like bronchoalveolar T-lymphocyte population in atopic asthma. N Engl J Med 1992; 326:298-304.
2. Burrows B, Martinez F D, Halonen M, Barbee R A, Cline M G. Association of asthma with serum IgE levels and skin-test reactivity to allergens. N Engl J Med 1989; 320:271-7.
3. Strunk R C, Bloomberg G R. Omalizumab for asthma. N Engl J Med 2006; 354: 2689-95.
4. Coffman R L, Correa-Oliviera R, Mocci S. Reversal of polarized T helper 1 and T helper 2 cell populations in murine leishmaniasis. Ciba Found Symp 1995; 195: 20-33.
5. Mocci S, Coffman R L. Induction of a Th2 population from a polarized Leishmania-specific Th1 population by in vitro culture with IL-4. J Immunol 1995; 154:3779-87.
6. Yang X O, Nurieva R, Martinez G J, Kang H S, Chung Y, Pappu B P, et al. Molecular antagonism and plasticity of regulatory and inflammatory T cell programs. Immunity 2008; 29:44-56.
7. Koenen H J, Smeets R L, Vink P M, van Rij ssen E, Boots A M, Joosten I. Human CD25highFoxp3pos regulatory T cells differentiate into IL-17-producing cells. Blood 2008; 112:2340-52.
8. Lee Y K, Turner H, Maynard C L, Oliver J R, Chen D, Elson C O, et al. Late developmental plasticity in the T helper 17 lineage. Immunity 2009; 30:92-107.
9. Wei G, Wei L, Zhu J, Zang C, Hu-Li J, Yao Z, et al. Global mapping of H3K4me3 and H3K27me3 reveals specificity and plasticity in lineage fate determination of differentiating CD41 T cells. Immunity 2009; 30:155-67.
10. Zhou L, Chong M M, Littman D R. Plasticity of CD41 T cell lineage differentiation. Immunity 2009; 30:646-55.
11. Cosmi L, Maggi L, Santarlasci V, Capone M, Cardilicchia E, Frosali F, et al. Identification of a novel subset of human circulating memory CD4(1) T cells that produce both IL-17A and IL-4. J Allergy Clin Immunol 2010; 125:222-30, e1-4.
12. Wang Y H, Voo K S, Liu B, Chen C Y, Uygungil B, Spoede W, et al. A novel subset of CD4(1) T(H)2 memory/effector cells that produce inflammatory IL-17 cytokine and promote the exacerbation of chronic allergic asthma. J Exp Med 2010; 207:2479-91.
13. Malmhall C, Bossios A, Radinger M, Sjostrand M, Lu Y, Lundback B, et al. Immunophenotyping of circulating T helper cells argues for multiple functions and plasticity of T cells in vivo in humans-possible role in asthma. PLoS One 2012; 7:e40012.
14. McKinley L, Alcorn J F, Peterson A, Dupont R B, Kapadia S, Logar A, et al. TH17 cells mediate steroid-resistant airway inflammation and airway hyperresponsiveness in mice. J Immunol 2008; 181:4089-97.
15. Pene J, Chevalier S, Preisser L, Venereau E, Guilleux M H, Ghannam S, et al. Chronically inflamed human tissues are infiltrated by highly differentiated Th17 lymphocytes. J Immunol 2008; 180:7423-30.
16. Barczyk A, Pierzchala W, Sozanska E. Interleukin-17 in sputum correlates with airway hyperresponsiveness to methacholine. Respir Med 2003; 97:726-33.
17. Bullens D M, Truyen E, Coteur L, Dilissen E, Hellings P W, Dupont L J, et al. IL-17 mRNA in sputum of asthmatic patients: linking T cell driven inflammation and granulocytic influx? Respir Res 2006; 7:135.
18. Molet S, Hamid Q, Davoine F, Nutku E, Taha R, Page N, et al. IL-17 is increased in asthmatic airways and induces human bronchial fibroblasts to produce cytokines. J Allergy Clin Immunol 2001; 108:430-8.
19. Al-Ramli W, Prefontaine D, Chouiali F, Martin J G, Olivenstein R, Lemiere C, et al. T(H)17-associated cytokines (IL-17A and IL-17F) in severe asthma. J Allergy Clin Immunol 2009; 123:1185-7.
20. Agache I, Ciobanu C, Agache C, Anghel M. Increased serum IL-17 is an independent risk factor for severe asthma. Respir Med 2010; 104:1131-7.
21. Wong C K, Ho C Y, Ko F W, Chan C H, Ho A S, Hui D S, et al. Proinflammatory cytokines (IL-17, IL-6, IL-18 and IL-12) and Th cytokines (IFN-gamma, IL-4, IL-10 and IL-13) in patients with allergic asthma. Clin Exp Immunol 2001; 125:177-83.
22. Vazquez-Tello A, Semlali A, Chakir J, Martin J G, Leung D Y, Eidelman D H, et al. Induction of glucocorticoid receptor-beta expression in epithelial cells of asthmatic airways by T-helper type 17 cytokines. Clin Exp Allergy 2010; 40:1312-22.
23. Good J T Jr, Kolakowski C A, Groshong S D, Murphy J R, Martin R J. Refractory asthma: importance of bronchoscopy to identify phenotypes and direct therapy. Chest 2012; 141:599-606.
24. Liang Q, Guo L, Gogate S, Karim Z, Hanifi A, Leung D Y, et al. IL-2 and IL-4 stimulate MEK1 expression and contribute to T cell resistance against suppression by TGF-beta and IL-10 in asthma. J Immunol 2010; 185:5704-13.
25. Thomas S Y, Lilly C M, Luster A D. Invariant natural killer T cells in bronchial asthma. N Engl J Med 2006; 354:2613-6.
26. Guo L, Chen C, Liang Q, Karim M Z, Gorska M M, Alam R. Nuclear translocation of MEK1 triggers a complex T cell response through the corepressor silencing mediator of retinoid and thyroid hormone receptor. J Immunol 2013; 190:159-67.
27. Kaplan M H, Grusby M J. Regulation of T helper cell differentiation by STAT molecules. J Leukoc Biol 1998; 64:2-5.
28. Yang X O, Panopoulos A D, Nurieva R, Chang S H, Wang D, Watowich S S, et al. STAT3 regulates cytokine-mediated generation of inflammatory helper T cells J Biol Chem 2007; 282:9358-63.
29. Kudo M, Melton A C, Chen C, Engler M B, Huang K E, Ren X, et al. IL-17A produced by alphabeta T cells drives airway hyper-responsiveness in mice and enhances mouse and human airway smooth muscle contraction. Nat Med 2012; 18: 547-54.
30. Kassel O, Sancono A, Kratzschmar J, Kreft B, Stassen M, Cato A C. Glucocorticoids inhibit MAP kinase via increased expression and decreased degradation of MKP-1. EMBO J 2001; 20:7108-16.
31. Tsitoura D C, Rothman P B. Enhancement of MEK/ERK signaling promotes glucocorticoid resistance in CD41 T cells. J Clin Invest 2004; 113:619-27.
32. Adcock I M, Lane S J, Brown C R, Lee T H, Barnes P J. Abnormal glucocorticoid receptor-activator protein 1 interaction in steroid-resistant asthma. J Exp Med 1995; 182:1951-8.
33. Mittelstadt P R, Ashwell J D. Inhibition of AP-1 by the glucocorticoid-inducible protein GILZ. J Biol Chem 2001; 276:29603-10.
34. Romanet-Manent S, Charpin D, Magnan A, Lanteaume A, Vervloet D. Allergic vs nonallergic asthma: what makes the difference? Allergy 2002; 57:607-13.
35. Robinson D, Hamid Q, Bentley A, Ying S, Kay A B, Durham S R. Activation of CD41 T cells, increased TH2-type cytokine mRNA expression, and eosinophil recruitment in bronchoalveolar lavage after allergen inhalation challenge in patients with atopic asthma. J Allergy Clin Immunol 1993; 92:313-24.
36. Krug N, Erpenbeck V J, Balke K, Petschallies J, Tschernig T, Hohlfeld J M, et al. Cytokine profile of bronchoalveolar lavage-derived CD4(1), CD8(1), and gammadelta T cells in people with asthma after segmental allergen challenge. Am J Respir Cell Mol Biol 2001; 25:125-31.
37. Thunberg S, Gafvelin G, Nord M, Gronneberg R, Grunewald J, Eklund A, et al. Allergen provocation increases TH2-cytokines and FOXP3 expression in the asthmatic lung. Allergy 2010; 65:311-8.
38. Walker C, Bauer W, Braun R K, Menz G, Braun P, Schwarz F, et al. Activated T cells and cytokines in bronchoalveolar lavages from patients with various lung diseases associated with eosinophilia. Am J Respir Crit Care Med 1994; 150: 1038-48.
39. Leung D Y, Martin R J, Szefler S J, Sher E R, Ying S, Kay A B, et al. Dysregulation of interleukin 4, interleukin 5, and interferon gamma gene expression in steroidresistant asthma. J Exp Med 1995; 181:33-40.
40. Huang S K, Xiao H Q, Kleine-Tebbe J, Paciotti G, Marsh D G, Lichtenstein L M, et al. IL-13 expression at the sites of allergen challenge in patients with asthma. J Immunol 1995; 155:2688-94.
41. Brightling C E, Symon F A, Birring S S, Bradding P, Pavord I D, Wardlaw A J. TH2 cytokine expression in bronchoalveolar lavage fluid T lymphocytes and bronchial submucosa is a feature of asthma and eosinophilic bronchitis. J Allergy Clin Immunol 2002; 110:899-905.
42. Bossley C J, Fleming L, Gupta A, Regamey N, Frith J, Oates T, et al. Pediatric severe asthma is characterized by eosinophilia and remodeling without T(H)2 cytokines. J Allergy Clin Immunol 2012; 129:974-82.e13.
43. Batra V, Musani A I, Hastie A T, Khurana S, Carpenter K A, Zangrilli J G, et al. Bronchoalveolar lavage fluid concentrations of transforming growth factor (TGF)-beta1, TGF-beta2, interleukin (IL)-4 and IL-13 after segmental allergen challenge and their effects on alpha-smooth muscle actin and collagen III synthesis by primary human lung fibroblasts. Clin Exp Allergy 2004; 34:437-44.
44. Zhu Z, Homer R J, Wang Z, Chen Q, Geba G P, Wang J, et al. Pulmonary expression of interleukin-13 causes inflammation, mucus hypersecretion, subepithelial fibrosis, physiologic abnormalities, and eotaxin production. J Clin Invest 1999; 103:779-88.
45. Striz I, Mio T, Adachi Y, Heires P, Robbins R A, Spurzem J R, et al. IL-4 induces ICAM-1 expression in human bronchial epithelial cells and potentiates TNFalpha. Am J Physiol 1999; 277:L58-64.
46. Woodruff P G, Modrek B, Choy D F, Jia G, Abbas A R, Ellwanger A, et al. T-helper type 2-driven inflammation defines major subphenotypes of asthma. Am J Respir Crit Care Med 2009; 180:388-95.
47. Hartenstein B, Teurich S, Hess J, Schenkel J, Schorpp-Kistner M, Angel P. Th2 cell-specific cytokine expression and allergen-induced airway inflammation depend on JunB. EMBO J 2002; 21:6321-9.
48. Glasmacher E, Agrawal S, Chang A B, Murphy T L, Zeng W, Vander Lugt B, et al. A genomic regulatory element that directs assembly and function of immunespecific AP-1-IRF complexes. Science 2012; 338:975-80.
49. Li P, Spolski R, Liao W, Wang L, Murphy T L, Murphy K M, et al. BATF-JUN is critical for IRF4-mediated transcription in T cells. Nature 2012; 490:543-6.
50. Ciofani M, Madar A, Galan C, Sellars M, Mace K, Pauli F, et al. A validated regulatory network for Th17 cell specification. Cell 2012; 151:289-303.
51. Fontanet P, Irala D, Alsina F C, Paratcha G, Ledda F. Pea3 transcription factor family members Etv4 and Etv5 mediate retrograde signaling and axonal growth of DRG sensory neurons in response to NGF. J Neurosci 2013; 33: 15940-51.
52. Li X, Newbern J M, Wu Y, Morgan-Smith M, Zhong J, Charron J, et al. MEK is a key regulator of gliogenesis in the developing brain. Neuron 2012; 75:1035-50.
53. Pham D, Sehra S, Sun X, Kaplan M H. The transcription factor Etv5 controls TH17 cell development and allergic airway inflammation. J Allergy Clin Immunol 2014; 134:204-14.
54. Irusen E, Matthews J G, Takahashi A, Barnes P J, Chung K F, Adcock I M. p38 Mitogen-activated protein kinase-induced glucocorticoid receptor phosphorylation reduces its activity: role in steroid-insensitive asthma. J Allergy Clin Immunol 2002; 109:649-57.
55. Ito K, Caramori G, Lim S, Oates T, Chung K F, Barnes P J, et al. Expression and activity of histone deacetylases in human asthmatic airways. Am J Respir Crit Care Med 2002; 166:392-6.
56. To Y, Ito K, Kizawa Y, Failla M, Ito M, Kusama T, et al. Targeting phosphoinositide-3-kinase-delta with theophylline reverses corticosteroid insensitivity in chronic obstructive pulmonary disease. Am J Respir Crit Care Med 2010; 182:897-904.
57. Jackson D J, Sykes A, Mallia P, Johnston S L. Asthma exacerbations: origin, effect, and prevention. J Allergy Clin Immunol 2011; 128:1165-74.
58. Johnston S L, Martin R J. Chlamydophila pneumoniae and Mycoplasma pneumoniae: a role in asthma pathogenesis? Am J Respir Crit Care Med 2005; 172: 1078-89.
59. To T, Shen S, Atenafu E G, Guan J, McLimont S, Stocks B, et al. The air quality health index and asthma morbidity: a population-based study. Environ Health Perspect 2013; 121:46-52.
60. Perez L, Declercq C, Iniguez C, Aguilera I, Badaloni C, Ballester F, et al. Chronic burden of near-roadway traffic pollution in 10 European cities (APHEKOM network). Eur Respir J 2013; 42:594-605.
61. Tabarani C M, Bonville C A, Suryadevara M, Branigan P, Wang D, Huang D, et al. Novel inflammatory markers, clinical risk factors and virus type associated with severe respiratory syncytial virus infection. Pediatr Infect Dis J 2013; 32:e437-42.
62. Thompson A M, Zanobetti A, Silverman F, Schwartz J, Coull B, Urch B, et al. Baseline repeated measures from controlled human exposure studies: associations between ambient air pollution exposure and the systemic inflammatory biomarkers IL-6 and fibrinogen. Environ Health Perspect 2010; 118:120-4.
63. Jackson D J, Sykes A, Mallia P, Johnston S L. Asthma exacerbations: origin, effect, and prevention. J Allergy Clin Immunol 2011; 128:1165-74.
64. Lemanske R F, Busse W W. Asthma: clinical expression and molecular mechanisms. J Allergy Clin Immunol 2010; 125(Suppl):S95-102.
65. Townley R G, Ryo Y U, Kolotkin B M, Kang B. Bronchial sensitivity to methacholine in current and former asthmatic and allergic rhinitis patients and control subjects. J Allergy Clin Immunol 1975; 56:429-42.
66. Shapiro G G, Furukawa C T, Pierson P E, Bierman C W. Methacholine bronchial challenge in children. J Allergy Clin Immunol 1982; 69:365-9.
67. Malo J L, Ghezzo H, D′Aquino C, L'Archeveque J, Cartier A, Chan-Yeung M. Natural history of occupational asthma: relevance of type of agent and other factors in the rate of development of symptoms in affected subjects. J Allergy Clin Immunol 1992; 90:937-44.
68. Moller D R, McKay R T, Bernstein I L, Brooks S M. Persistent airways disease caused by toluene diisocyanate. Am Rev Respir Dis 1986; 134:175-6.
69. Kips J C, Anderson G P, Fredberg J J, Herz U, Inman M D, Jordana M, et al. Murine models of asthma. Eur Respir J 2003; 22:374-82.
70. Kumar R K, Foster P S. Modeling allergic asthma in mice: pitfalls and opportunities. Am J Respir Cell Mol Biol 2002; 27:267-72.
71. Duez C, Tomkinson A, Shultz L D, Bratton D L, Gelfand E W. Fas deficiency delays the resolution of airway hyperresponsiveness after allergen sensitization and challenge. J Allergy Clin Immunol 2001; 108:547-56.
72. Haworth O, Cernadas M, Levy B D. NK cells are effectors for resolvin E1 in the timely resolution of allergic airway inflammation. J Immunol 2011; 186: 6129-35.
73. Leech M D, Benson R A, De Vries A, Fitch P M, Howie S E. Resolution of Der p1-induced allergic airway inflammation is dependent on CD41CD251Foxp31 regulatory cells. J Immunol 2007; 179:7050-8.
74. Kumar R K, Herbert C, Kasper M. Reversibility of airway inflammation and remodeling following cessation of antigenic challenge in a model of chronic asthma. Clin Exp Allergy 2004; 34:1796-802.
75. Schramm C M, Puddington L, Wu C, Guernsey L, Gharaee-Kermani M, Phan S H, et al. Chronic inhaled ovalbumin exposure induces antigen-dependent but not antigen-specific inhalational tolerance in a murine model of allergic airway disease. Am J Pathol 2004; 164:295-304.
76. Chen Z G, Zhang T T, Li H T, Chen F H, Zou X L, Ji J Z, et al. Neutralization of TSLP inhibits airway remodeling in a murine model of allergic asthma induced by chronic exposure to house dust mite. PLoS One 2013; 8:e51268.
77. Henderson W R Jr, Tang L O, Chu S J, Tsao S M, Chiang G K, Jones F, et al. A role for cysteinyl leukotrienes in airway remodeling in a mouse asthma model. Am J Respir Crit Care Med 2002; 165:108-16.
78. Johnson J R, Wiley RE, Fattouh R, Swirski F K, Gajewska B U, Coyle A J, et al. Continuous exposure to house dust mite elicits chronic airway inflammation and structural remodeling. Am J Respir Crit Care Med 2004; 69:378-85.
79. Goplen N, Karim M Z, Liang Q, Gorska M M, Rozario S, Guo L, et al. Combined sensitization of mice to extracts of dust mite, ragweed, and Aspergillus species breaks through tolerance and establishes chronic features of asthma. J Allergy Clin Immunol 2009; 123:925-32.e11.
80. Moro K, Yamada T, Tanabe M, Takeuchi T, Ikawa T, Kawamoto H, et al. Innate production of T(H)2 cytokines by adipose tissue-associated c-Kit(1)Sca-1(1) lymphoid cells. Nature 2010; 463:540-4.
81. Neill D R, Wong S H, Bellosi A, Flynn R J, Daly M, Langford T K, et al. Nuocytes represent a new innate effector leukocyte that mediates type-2 immunity. Nature 2010; 464:1367-70.
82. Saenz S A, Siracusa M C, Perrigoue J G, Spencer S P, Urban J F Jr, Tocker J E, et al. IL25 elicits a multipotent progenitor cell population that promotes T(H)2 cytokine responses. Nature 2010; 464:1362-6.
83. Mj€osberg J M, Trifari S, Crellin N K, Peters C P, van Drunen C M, Piet B, et al. Human IL-25- and IL-33-responsive type 2 innate lymphoid cells are defined by expression of CRTH2 and CD161. Nat Immunol 2011; 12:1055-62.
84. Spits H, Di Santo J P. The expanding family of innate lymphoid cells: regulators and effectors of immunity and tissue remodeling. Nat Immunol 2011; 12:21-7.
85. Barlow J L, Peel S, Fox J, Panova V, Hardman C S, Camelo A, et al. IL-33 is more potent than IL-25 in provoking IL-13-producing nuocytes (type 2 innate lymphoid cells) and airway contraction. J Allergy Clin Immunol 2013; 132: 933-41.
86. Bartemes K R, Iijima K, Kobayashi T, Kephart G M, McKenzie A N, Kita H. IL-33-responsive lineage-CD251 CD44(hi) lymphoid cells mediate innate type 2 immunity and allergic inflammation in the lungs. J Immunol 2012; 88:1503-13.
87. Doherty T A, Khorram N, Chang J E, Kim H K, Rosenthal P, Croft M, et al. STAT6 regulates natural helper cell proliferation during lung inflammation initiated by Alternaria. Am J Physiol Lung Cell Mol Physiol 2012; 303:L577-88.
88. Halim T Y, Krauss R H, Sun A C, Takei F. Lung natural helper cells are a critical source of Th2 cell-type cytokines in protease allergen-induced airway inflammation. Immunity 2012; 36:451-63.
89. Kim H Y, Chang Y J, Subramanian S, Lee H H, Albacker L A, Matangkasombut P, et al. Innate lymphoid cells responding to IL-33 mediate airway hyperreactivity independently of adaptive immunity. J Allergy Clin Immunol 2012; 129:216-27, e211-6.
90. Petersen B C, Budelsky A L, Baptist A P, Schaller M A, Lukacs N W. Interleukin-25 induces type 2 cytokine production in a steroid-resistant interleukin-17RB1 myeloid population that exacerbates asthmatic pathology. Nat Med 2012; 18: 751-8.
91. Doherty T A, Soroosh P, Broide D H, Croft M. CD41 cells are required for chronic eosinophilic lung inflammation but not airway remodeling. Am J Physiol Lung Cell Mol Physiol 2009; 296:L229-35.
92. Wong S H, Walker J A, Jolin H E, Drynan L F, Hams E, Camelo A, et al. Transcription factor RORalpha is critical for nuocyte development. Nat Immunol 2012; 13:229-36.
93. Chu D K, Llop-Guevara A, Walker T D, Flader K, Goncharova S, Boudreau J E, et al. IL-33, but not thymic stromal lymphopoietin or IL-25, is central to mite and peanut allergic sensitization. J Allergy Clin Immunol 2013; 131:187-200, e181-8.
94. Lefranc, ais E, Roga S, Gautier V, Gonzalez-de-Peredo A, Monsarrat B, Girard J P, et al. IL-33 is processed into mature bioactive forms by neutrophil elastase and cathepsin G. Proc Natl Acad Sci USA 2012; 109:1673-8.
95. Chang Y J, Kim H Y, Albacker L A, Baumgarth N, McKenzie A N, Smith D E, et al. Innate lymphoid cells mediate influenza-induced airway hyper-reactivity independently of adaptive immunity. Nat Immunol 2011; 12:631-8.
96. Kondo Y, Yoshimoto T, Yasuda K, Futatsugi-Yumikura S, Morimoto M, Hayashi N, et al. Administration of IL-33 induces airway hyperresponsiveness and goblet cell hyperplasia in the lungs in the absence of adaptive immune system. Int Immunol 2008; 20:791-800.
97. Kurowska-Stolarska M, Kewin P, Murphy G, Russo R C, Stolarski B, Garcia C C, et al. IL-33 induces antigen-specific IL-51 T cells and promotes allergic-induced airway inflammation independent of IL-4. J Immunol 2008; 181:4780-90.
98. Hardman C S, Panova V, McKenzie A N. IL-33 citrine reporter mice reveal the temporal and spatial expression of IL-33 during allergic lung inflammation. Eur J Immunol 2013; 43:488-98.
99. Yagami A, Orihara K, Morita H, Futamura K, Hashimoto N, Matsumoto K, et al. IL-33 mediates inflammatory responses in human lung tissue cells. J Immunol 2010; 185:5743-50.
100. Masamune A, Watanabe T, Kikuta K, Satoh K, Kanno A, Shimosegawa T. Nuclear expression of interleukin-33 in pancreatic stellate cells. Am J Physiol Gastrointest Liver Physiol 2010; 299:G821-32.
101. Zhang L, Lu R, Zhao G, Pflugfelder S C, Li D Q. TLR-mediated induction of proallergic cytokine IL-33 in ocular mucosal epithelium. Int J Biochem Cell Biol 2011; 43:1383-91.
102. Shaw J L, Fakhri S, Citardi M J, Porter P C, Corry D B, Kheradmand F, et al. IL-33-responsive innate lymphoid cells are an important source of IL-13 in chronic rhinosinusitis with nasal polyps. Am J Respir Crit Care Med 2013; 188:432-9.
103. Corry D B, Grfunig G, Hadeiba H, Kurup V P, Warnock M L, Sheppard D, et al. Requirements for allergen-induced airway hyperreactivity in T and B cell-deficient mice. Mol Med 1998; 4:344-55.
104. Byers D E, Alexander-Brett J, Patel A C, Agapov E, Dang-Vu G, Jin X, et al. Long-term IL-33-producing epithelial progenitor cells in chronic obstructive lung disease. J Clin Invest 2013; 123:3967-82.
105. Chang D E, Leung S, Atkinson M R, Reifler A, Forger D, Ninfa A J. Building biological memory by linking positive feedback loops. Proc Natl Acad Sci USA 2010; 107:175-80.
106. Shin S Y, Rath O, Zebisch A, Choo S M, Kolch W, Cho K H. Functional roles of multiple feedback loops in extracellular signal-regulated kinase and Wnt signaling pathways that regulate epithelial-mesenchymal transition. Cancer Res 2010; 70:6715-24.
107. Xiong W, Ferrell J E Jr. A positive-feedback-based bistable ‘memory module’ that governs a cell fate decision. Nature 2003; 426:460-5.
108. Markevich N I, Hoek J B, Kholodenko B N. Signaling switches and bistability arising from multisite phosphorylation in protein kinase cascades. J Cell Biol 2004; 164:353-9.
109. Srividhya J, Li Y, Pomerening J R. Open cascades as simple solutions to providing ultrasensitivity and adaptation in cellular signaling. Phys Biol 2011; 8:046005.
110. LiuW, Tundwal K, LiangQ, GoplenN, Rozario S, QuayumN, et al. Establishment of extracellular signal-regulated kinase 1/2 bistability and sustained activation through Sprouty 2 and its relevance for epithelial function. Mol Cell Biol 2010; 30:1783-99.
111. Liu W, Liang Q, Balzar S, Wenzel S, Gorska M M, Alam R. Cell-specific activation profile of extracellular signal-regulated kinase 1/2, Jun N-terminal kinase, and p38 mitogen-activated protein kinases in asthmatic airways. J Allergy Clin Immunol 2008; 121:893-902.e2.
112. Prefontaine D, Lajoie-Kadoch S, Foley S, Audusseau S, Olivenstein R, Halayko A J, et al. Increased expression of IL-33 in severe asthma: evidence of expression by airway smooth muscle cells. J Immunol 2009; 183:5094-103.
113. Prefontaine D, Nadigel J, Chouiali F, Audusseau S, Semlali A, Chakir J, et al. Increased IL-33 expression by epithelial cells in bronchial asthma. J Allergy Clin Immunol 2010; 125:752-4.
114. Bartemes K R, Kephart G M, Fox S J, Kita H. Enhanced innate type 2 immune response in peripheral blood from patients with asthma. J Allergy Clin Immunol 2014; 134:671-8.e4.
115. Goplen N, et al. ERK1 is important for Th2 differentiation and development of experimental asthma. FASEB J 2012; 26:1934-1945.
116. Haile S, Lefort J, Joseph D, Gounon P, Huerre M, Vargaftig B B. Mucous-cell metaplasia and inflammatory-cell recruitment are dissociated in allergic mice after antibody- and drug-dependent cell depletion in a murine model of asthma. Am J Respir Cell Mol Biol. 1999; 20:891-902.
117. Goplen N, Karim M Z, Liang Q, Gorska M M, Rozario S, Guo L, et al. Combined sensitization of mice to extracts of dust mite, ragweed, and Aspergillus species breaks through tolerance and establishes chronic features of asthma. J Allergy Clin Immunol 2009; 123:925-32.e11.
118. Gorska M M, et al. MK2 controls the level of negative feedback in the NF-kappaB pathway and is essential for vascular permeability and airway inflammation. J Exp Med2007; 204:1637-1652.
119. Good J T, Kolakowski C A, Groshong S D, Murphy J R, Martin R J. Refractory asthma: importance of bronchoscopy to identify phenotypes and direct therapy. Chest 2012; 141:599-606.

1.-9. (canceled)

10. A method of treating a subject having persistent asthma comprising
a. obtaining a sample from the subject;
b. determining the frequency of Type-2 cytokine-producing innate lymphoid (ILC2) cells in the sample;
c. comparing the frequency from step (b) to a control level, wherein an increased frequency from the subject as compared to the control identifies the subject as having persistent asthma; and
d. administering to the subject a compound selected from the group consisting of a bronchodilator, corticosteroid, leukotriene antagonist, anti-cytokine antibody, anti-cytokine receptor antibody, anti-IgE antibody, an antibiotic, a phosphodiesaterease inhibitor, an anti-MEK compound and combinations thereof for treating the subject.

11. The method of claim 10, wherein the greater the frequency of ILC2 cells as compared to the control, indicates greater severity of the persistent asthma.

12. The method of claim 10, further comprising determining the expression level of interleukin 33 (IL33) in the sample from the subject and comparing the expression level to an IL33 expression level from a control, wherein an increased level of IL33 from the subject as compared to IL33 expression level from the control indicates greater severity of the persistent asthma.

13. The method of claim 10, wherein the frequency of ILC2 cells in the sample is determined by flow cytometry.

14. The method of claim 13, wherein the expression level of IL33 is determined by ELISA.

15. The method of claim 10, wherein the sample from the subject is selected from the group consisting of bronchoalveolar lavage fluid (BAL), peripheral blood, nasal washing and induced sputum.

16.-20. (canceled)


SEQUENCE LISTING (20 sequences)

SEQ ID NO: 1 - DNA, 22 bp, Artificial Sequence
  Synthetic Primer mIL4 F
ttgagagagatcatcggcattt

SEQ ID NO: 2 - DNA, 22 bp, Artificial Sequence
  Synthetic Primer mIL4 R
ctcactctctgtggtgttcttc

SEQ ID NO: 3 - DNA, 21 bp, Artificial Sequence
  Synthetic Primer mIL7 F
cacactcacgtccagatttag

SEQ ID NO: 4 - DNA, 19 bp, Artificial Sequence
  Synthetic Primer mIL7 R
tcctagcctgccttagatc

SEQ ID NO: 5 - DNA, 18 bp, Artificial Sequence
  Synthetic Primer mIL25 F
ccagcaaagagcaagaac

SEQ ID NO: 6 - DNA, 18 bp, Artificial Sequence
  Synthetic Primer mIL25 R
ttcaagtccctgtccaac

SEQ ID NO: 7 - DNA, 19 bp, Artificial Sequence
  Synthetic Primer mIL33 F
tcccaacagaagaccaaag

SEQ ID NO: 8 - DNA, 19 bp, Artificial Sequence
  Synthetic Primer mIL33 R
gatactgccaagcaaggat

SEQ ID NO: 9 - DNA, 19 bp, Artificial Sequence
  Synthetic Primer mTSLP F
cttctcaggagcctcttca

SEQ ID NO: 10 - DNA, 18 bp, Artificial Sequence
  Synthetic Primer mTSLP R
agccagggataggattga

SEQ ID NO: 11 - DNA, 20 bp, Artificial Sequence
  Synthetic Primer mST-2 F
gtgacaccttacaaaacccg

SEQ ID NO: 12 - DNA, 19 bp, Artificial Sequence
  Synthetic Primer mST-2 R
tcaagaacgtcgggcagag

SEQ ID NO: 13 - DNA, 21 bp, Artificial Sequence
  Synthetic Primer mGAPDH F
acggccgcatcttcttgtgca

SEQ ID NO: 14 - DNA, 20 bp, Artificial Sequence
  Synthetic Primer mGAPDH R
aatggcagccctggtgacca

SEQ ID NO: 15 - DNA, 21 bp, Artificial Sequence
  Synthetic Primer hIL33 F
gtgacggtgttgatggtaaga

SEQ ID NO: 16 - DNA, 21 bp, Artificial Sequence
  Synthetic Primer hIL33 R
ctccacagagtgttccttgtt

SEQ ID NO: 17 - DNA, 22 bp, Artificial Sequence
  Synthetic Primer hST-2 F
aacgagttaccaatacttgctc

SEQ ID NO: 18 - DNA, 20 bp, Artificial Sequence
  Synthetic Primer hST-R
caggcactattgcttctggg

SEQ ID NO: 19 - DNA, 21 bp, Artificial Sequence
  Synthetic Primer h18S F
ctgagaaacggctaccacatc

SEQ ID NO: 20 - DNA, 22 bp, Artificial Sequence
  Synthetic Primer h18S R
gcctcgaaagagtcctgtattg